(19) United States
(12) Patent Application Publication
(10) Pub. No.: US 2026/0077061 A1
(43) Pub. Date: Mar. 19, 2026

(54) COMPOSITIONS AND METHODS FOR IN VIVO NUCLEASE-MEDIATED GENE TARGETING FOR THE TREATMENT OF GENETIC DISORDERS IN ADULT PATIENTS

(71) Applicant: The Trustees of the University of Pennsylvania, Philadelphia, PA (US)

(72) Inventors: James M. Wilson, Philadelphia, PA (US); Lili Wang, Phoenixville, PA (US)

(73) Assignee: The Trustees of the University of Pennsylvania, Philadelphia, PA (US)

(21) Appl. No.: 18/994,165

(22) PCT Filed: Jul. 14, 2023
(86) PCT No.: PCT/US2023/070228

§ 371 Date: Jan. 14, 2025

Related U.S. Application Data
(60) Provisional application No. 63/368,447, filed on Jul. 14, 2022.
(60) Provisional application No. 63/381,809, filed on Nov. 1, 2022.
(60) Provisional application No. 63/502,270, filed on May 15, 2023.

(51) Int. Cl. A61K 48/00; A61K 38/48; A61P 7/04; C07K 14/005; C12N 9/22; C12N 15/11; C12N 15/86; C12N 15/88; C12N 15/90

(52) U.S. Cl. CPC A61K 48/0058; A61K 38/4846; A61K 48/0075; A61K 48/0083; A61P 7/04; C07K 14/005; C12N 9/226; C12N 15/111; C12N 15/86; C12N 15/88; C12N 15/907; C12Y 304/21022; C12Y 304/21061; C12N 2310/20; C12N 2750/14122; C12N 2750/14143

(57) ABSTRACT

A dual component system for treating a genetic disorder is provided. The system includes (a) a gene editing vector comprising an expression cassette comprising a nucleic acid sequence encoding a nuclease and regulatory sequences that direct expression of the nuclease in a target cell comprising a PCSK9 gene; and (b) a donor vector comprising a nucleic acid sequence encoding an exogenous product for expression from the PCSK9 locus, wherein the inserted nucleic acid sequence does not encode PCSK9, wherein the system further comprises sequences that direct the nuclease to specifically targets the native PCSK9 gene locus; and wherein the native PCSK9 in the target cell is optionally ablated or reduced post-dosing with the dual component system.

### REFERENCE TO AN ELECTRONIC SEQUENCE LISTING

The contents of the electronic sequence listing (UPN-23-10151.PCT.xml; Size: 271 kb; and Date of Creation: Jul. 13, 2023) is herein incorporated by reference in its entirety.

### BACKGROUND OF THE INVENTION

Site-specific nucleases (such as CRISPR-Cas9 or meganucleases) generate double strand breaks (DSBs) in the chromosome, leading to DNA repair. In the presence of donor DNA, homology directed repair (HDR) occurs and replaces genetic information in the chromosome with new information from the donor gene.

Homology-directed repair (HDR) is a process where a DNA double-strand break (DSB) is repaired by homologous recombination using a DNA template. This template can come from within the cell during late S phase or G2 phase of the cell cycle, when sister chromatids are available prior to the completion of mitosis. Additionally, exogenous repair templates can be delivered into a cell, most often in the form of a synthetic, single-strand DNA donor oligo or donor plasmid, to generate a precise change in the genome.

Safe harbor sites (SHS) are genomic loci where genes or other genetic elements can be safely inserted and expressed. These SHS are critical for effective human disease gene therapies: for investigating gene structure, function and regulation; and for cell marking and tracking.

What are needed are improved compositions and methods for gene editing.

### SUMMARY OF THE INVENTION

Provided herein are compositions, methods, systems, and kits for gene editing, which allow knockdown or ablation of the native PCSK9 gene and insertion and/or expression of an exogenous transgene in the PCSK9 gene locus.

In a first aspect, provided herein is a system for treating a genetic disorder. The system includes a gene editing component comprising an expression cassette comprising a nucleic acid sequence encoding a nuclease that targets the PCSK9 gene and regulatory sequences that direct expression of the nuclease in a target cell comprising the PCSK9 gene. The system further includes a donor vector comprising a transgene cassette comprising a nucleic acid sequence encoding a transgene and regulatory sequences that direct expression of the transgene in the target cell, the donor vector further comprising homology-directed recombination (HDR) arms 5′ and 3′ to the transgene cassette, wherein the transgene is not PCSK9. The nuclease targets the PCSK9 gene. In some embodiments, the nuclease targets PCSK9 exon 7. In some embodiments, the meganuclease is the ARCUS meganuclease.

In some embodiments, the gene editing component comprises a sequence that encodes a Cas9. In certain embodiments, the gene editing vector further comprises sequence that encodes a sgRNA comprising an at least 20 nucleotide seed region, wherein the sgRNA specifically binds to a target site in the PCSK9 gene, said target site being 5′ to a protospacer-adjacent motif (PAM) that is specifically recognized by the Cas9.

In other embodiments, the donor vector further comprises sequences that encode a sgRNA comprising an at least 20 nucleotide seed region, wherein the sgRNA specifically binds to a target site in the PCSK9 gene, said target site being 5′ to a protospacer-adjacent motif (PAM) that is specifically recognized by the Cas9.

In another aspect, provided herein is a system for treating a genetic disorder. The system includes a gene editing component comprising a nucleic acid sequence encoding a nuclease that targets the PCSK9 gene. The system further includes a donor vector comprising a transgene cassette comprising a nucleic acid sequence encoding a transgene and regulatory sequences that direct expression of the transgene in the target cell, the donor vector further comprising homology-directed recombination (HDR) arms 5′ and 3′ to the transgene cassette, wherein the transgene is not PCSK9. The nuclease targets the PCSK9 gene. In certain embodiments, the gene editing component is provided in a lipid nanoparticle.

In some embodiments, the gene editing component comprises a sequence that encodes a Cas9. In certain embodiments, the gene editing vector further comprises a sequence that encodes a sgRNA comprising an at least 20 nucleotide seed region, wherein the sgRNA specifically binds to a target site in the PCSK9 gene, said target site being 5′ to a protospacer-adjacent motif (PAM) that is specifically recognized by the Cas9.

In other embodiments, the donor vector further component comprises a sequence that encodes a sgRNA comprising an at least 20 nucleotide seed region, wherein the sgRNA specifically binds to a target site in the PCSK9 gene, said target site being 5′ to a protospacer-adjacent motif (PAM) that is specifically recognized by the Cas9.

In certain embodiments, the transgene relates to a liver metabolic disorder. In certain embodiments, the transgene is FIX, OTC, PAH, ASSI, or LDLR.

In certain embodiments, the vectors are adeno-associated viral (AAV) vectors, and the vectors comprise AAV 5′ ITRs and AAV 3′ ITRs.

In another embodiment, the dual component system for treating a genetic disorder includes a gene editing AAV comprising an AAV capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and a donor AAV vector comprising an AAV capsid and a second vector genome comprising: a 5′ITR, a 5′ homology directed recombination (HDR) arm, a transgene and regulatory sequences that direct expression of the transgene in the target cell, a 3′ HDR arm, and a 3′ ITR, wherein the transgene does not encode PCSK9.

In another embodiment, the dual component system for treating a genetic disorder includes a gene editing AAV comprising an AAV capsid and a first vector genome comprising a 5′ ITR, a 5′ nuclear localization signal (NLS), a sequence encoding a Cas9 and regulatory sequences that direct expression of the saCas9 in a target cell comprising the PCSK9 gene, a 3′ NLS, and a 3′ ITR; and a donor AAV vector comprising an AAV capsid and a second vector genome comprising: a 5′ITR, a 5′ homology directed recombination (HDR) arm, a transgene and regulatory sequences that direct expression of the transgene in the target cell, a 3′ HDR arm, a U6 promoter, a sgRNA comprising at least 20 nucleotides that specifically bind to a target site in the PCSK9 gene, said target site being 5′ to a protospacer-adjacent motif (PAM) that is specifically recognized by the Cas9, and a 3′ ITR, wherein the transgene does not encode PCSK9.

In yet another embodiment, the dual component system for treating a genetic disorder includes a gene editing AAV vector comprising an AAV capsid and a first vector genome comprising a 5′ ITR, a U6 promoter, a sgRNA comprising at least 20 nucleotides that specifically bind to a target site in the PCSK9 gene, said target site being 5′ to a protospacer-adjacent motif (PAM) that is specifically recognized by the Cas9, a 5′ nuclear localization signal (NLS), a sequence encoding a Cas9 and regulatory sequences that direct expression of the Cas9 in a target cell comprising the PCSK9 gene, a 3′ NLS, and a 3′ ITR; and a donor AAV vector comprising an AAV capsid and a second vector genome comprising: a 5′ITR, a 5′ homology directed recombination (HDR) arm, a transgene and regulatory sequences that direct expression of the transgene in the target cell, a 3′ HDR arm, and a 3′ ITR.

In some embodiments, the gene editing AAV vector and the donor AAV vector have the same AAV capsid. In other embodiments, the gene editing AAV vector and the donor AAV vector have different AAV capsids. In some embodiments, the AAV capsid is selected from AAV8, AAV9, rh10, AAV6.2, AAV3B, hu37, rh79, and rh64.

In another aspect, a method of treating a disorder in humans by co-administering the dual component system as described herein, is provided.

In another aspect, a method of treating a genetic disorder in a subject is provided, the method includes co-administering to the subject having a the disorder a gene editing AAV vector comprising a sequence encoding a nuclease and regulatory sequences that direct expression of the nuclease in a target cell comprising a PCSK9 gene; and a donor AAV vector comprising a transgene and regulatory sequences that direct expression of the transgene in the target cell, the donor vector further comprising homology-directed recombination (HDR) arms 5′ and 3′ to the transgene cassette, wherein the transgene is not PCSK9. In certain embodiments, the disorder is hemophilia B. In one embodiment, the subject is a neonate.

In another aspect, a system for treating genetic disorders is provided. The system includes a lipid nanoparticle (LNP) comprising a mRNA sequence encoding a nuclease that targets the PCSK9 gene; and a donor AAV vector comprising a transgene and regulatory sequences which direct its expression in the target cell, the donor vector further comprising a homology-directed recombination (HDR) arms 5′ and 3′ to the transgene, wherein the transgene is not PCSK9. In some embodiments, the nuclease targets PCSK9 exon 7. In some embodiments, the meganuclease is the ARCUS meganuclease.

In other embodiments, the gene editing vector encodes a Cas9. In certain embodiments, the gene editing vector further encodes a sgRNA comprising at least 20 nucleotides, which specifically binds to a target site in the PCSK9 gene, said target site being 5′ to a protospacer-adjacent motif (PAM) that is specifically recognized by the Cas9. In some embodiments, where the system includes an LNP, the LNP comprises both the Cas9 coding sequence and gRNA.

In other embodiments, the donor vector further encodes a sgRNA comprising an at least 20 nucleotide seed region, wherein the sgRNA specifically binds to a target site in the PCSK9 gene, said target site being 5′ to a protospacer-adjacent motif (PAM) that is specifically recognized by the Cas9.

In yet another aspect, a dual component system for treating a genetic disorder is provided. The system includes a gene editing vector comprising an expression cassette comprising a nucleic acid sequence encoding a nuclease and regulatory sequences that direct expression of the nuclease in a target cell comprising a PCSK9 gene; and a donor vector comprising a nucleic acid sequence encoding an exogenous product for expression from the PCSK9 locus, wherein the inserted nucleic acid sequence does not encode PCSK9, wherein the system further comprises sequences that direct the nuclease to specifically targets the native PCSK9 gene locus; and wherein the native PCSK9 in the target cell is optionally ablated or reduced post-dosing with the dual component system.

In yet another aspect, a method of treating a patient is provided, using a system as described herein, wherein the patient's native PCSK9 expression levels are reduced and wherein the patient expresses the exogenous product.

Other aspects and advantages of the invention will be apparent from the following detailed description of the invention.

### BRIEF DESCRIPTION OF THE DRAWINGS

FIG. 1 shows a schematic representation of the rhPCSK9 locus showing the donor splice site within exon 7, and a HDR donor vector comprising a donor template of interest, e.g., hFIX, hOTC.

FIG. 2 shows a timeline for a pilot study comprising an hFIX mini-gene knock-in in PCSK9 locus by ARCUS2 or SaCas9 in newborn NHPs.

FIGS. 3A to 3C show a schematic representation for a dual AAV vector system for SaCas9- or ARCUS-mediated gene correction. FIG. 3A shows a schematic representation for a dual AAVhu37 vector system for ARCUS2-mediated gene correction, wherein the AAVhu37-donor vector comprises an hOTC donor template sequence. FIG. 3B shows a schematic representation for a dual AAVhu37 vector system for SaCas9-mediated gene correction (trans: AAVhu37-SaCas9), wherein the expression cassettes for SaCas9 and sgRNA are in two separate vectors, and AAVhu37.sgRNA-donor vector comprises an hOTC donor template sequence and a U6.sgRNA cassette. FIG. 3C shows a schematic representation for a dual AA Vhu37 vector system for SaCas9-mediated gene correction (cis: AAVhu37.PCSK9-sgRNA.SaCas9), wherein the expression cassettes for SaCas9 and sgRNA are in the same vector, and the hOTC donor vector is in a separate vector.

FIGS. 4A to 4H show an in vivo test of nuclease-mediated gene targeting in newborn NHPs. Animals were administered 1×10¹³ GC/kg of AAVhu37.ARCUS2.WPRE and 3×10¹³ GC/kg of AAVhu37.hFIXco-HDR or 1×10¹³ GC/kg of AAVhu37.SaCas9.WPRE and 3×10¹³ GC/kg of AAVhu37.hFIXco-HDR.U6.sgR or 1×10¹³ GC/kg of AAVhu37.GFP.WPRE and 3×10¹³ GC/kg of AAVhu37.hFIXco-HDR.U6.sgR, as shown in FIGS. 4A, 4B and 5G. FIG. 4C shows hFIX levels at the indicated timepoints (plotted as ng/mL) in newborn NHPs. FIG. 4D shows PCSK9 levels at the indicated timepoints (plotted as percentage of baseline at day 0) in newborn NHPs. FIG. 4E shows ALT (Alanine Aminotransferase) levels at the indicated timepoints (plotted as U/L) in newborn NHPs. FIG. 4F shows anti-FIX IgG levels at the indicated timepoints (plotted as dilution factor, 1/dilution) in newborn NHPs. FIG. 4G shows PCSK9 levels at the indicated timepoints (plotted as ng/ml) in newborn NHPs. FIG. 4H shows weight as measured (plotted as g) in newborn NHPs.

FIGS. 5A to 5H show the results of the in vivo test described for FIG. 4, administered to 3-month-old infant NHPs. FIG. 5A shows hFIX levels at the indicated timepoints (plotted as ng/mL) in infant NHPs. FIG. 5B shows PCSK9 levels at the indicated timepoints (plotted as percentage of baseline at day 0) in infant NHPs. FIG. 5C shows ALT (Alanine Aminotransferase) levels at the indicated timepoints (plotted as U/L) in infant NHPs. FIG. 5D shows anti-FIX IgG levels at the indicated timepoints (plotted as dilution factor, 1/dilution) in infant NHPs. FIG. 5E shows PCSK9 levels at the indicated timepoints (plotted as ng/mL) in infant NHPs. FIG. 5F shows weight as measured at the indicated timepoints (plotted as g) in infant NHPs. FIG. 5G is a summary table showing data from the experiment described in FIGS. 4A-5G. FIG. 5H shows a comparison of various data between newborn and infant NHPs tested.

FIGS. 6A to 6E show vector transduction (GC) and transgene expression in liver biopsies samples collected at various days post treatment in NHPs treated as described for FIGS. 4A-4H. FIG. 6A shows vector transduction levels in liver biopsies samples, plotted as AAV genome copies (GC) per diploid cell. FIG. 6B shows relative expression of transgene RNA in liver biopsies samples. FIG. 6C shows dual in situ hybridization (ISH) using specific probes to detect FIX and ARCUS in liver biopsies. FIG. 6D shows digitized ISH images used for quantification of transduction percentage. FIG. 6E shows transduction efficiency of FIX transgene as quantified by ISH, and plotted as percent transduction.

FIGS. 7A to 7L show dual in situ hybridization (ISH) using specific probes to detect FIX and ARCUS in liver biopsies collected at 84 days post treatment in NHPs: showed at various magnification views (NHPs treated with AAVhu37.ARCUS2 and AAVhu37.Donor-HDR-hFIX). FIG. 7A shows ISH-detected ARCUS in liver biopsies, viewed at 4× magnification. FIG. 7B shows ISH-detected hFIX in liver biopsies, viewed at 4× magnification. FIG. 7C shows overlay image of ISH-detected ARCUS and hFIX, viewed at 4× magnification. FIG. 7D shows ISH-detected ARCUS and hFIX as an overlayed image with DAPI (staining for nuclei), viewed at 4× magnification. FIG. 7E shows ISH-detected ARCUS in liver biopsies, viewed at 10× magnification. FIG. 7F shows ISH-detected hFIX in liver biopsies, viewed at 10× magnification. FIG. 7G shows overlay image of ISH-detected ARCUS and hFIX, viewed at 10× magnification. FIG. 7H shows ISH-detected ARCUS and hFIX as an overlayed image with DAPI (staining for nuclei), viewed at 10× magnification. FIG. 7I shows ISH-detected ARCUS expression in liver biopsies, viewed at 20× magnification. FIG. 7J shows ISH-detected hFIX in liver biopsies, viewed at 20× magnification. FIG. 7K shows overlay image of ISH-detected ARCUS and hFIX, viewed at 20× magnification. FIG. 7L shows ISH-detected ARCUS and hFIX as an overlayed image with DAPI (staining for nuclei), viewed at 20× magnification.

FIGS. 8A to 8M show dual in situ hybridization (ISH) using specific probes to detect FIX and ARCUS in liver biopsies collected at 84 days post treatment in NHPs: showed at various magnification views (NHPs treated with AAVhu37.EGFP and AAVhu37.Donor-HDR-hFIX.U6.sgR). FIG. 8A shows ISH-detected GFP-WRPE in liver biopsies, viewed at 4× magnification. FIG. 8B shows ISH-detected hFIX in liver biopsies, viewed at 4× magnification. FIG. 8C shows overlay image of ISH-detected GFP-WRPE and hFIX, viewed at 4× magnification. FIG. 8D shows ISH-detected GFP-WRPE and hFIX as an overlayed image with DAPI (staining for nuclei), viewed at 4× magnification. FIG. 8E shows ISH-detected GFP-WRPE in liver biopsies, viewed at 10× magnification. FIG. 8F shows ISH-detected hFIX in liver biopsies, viewed at 10× magnification. FIG. 8G shows overlay image of ISH-detected GFP-WRPE and hFIX, viewed at 10× magnification. FIG. 8H shows ISH-detected GFP-WRPE and hFIX as an overlayed image with DAPI (staining for nuclei), viewed at 10× magnification. FIG. 8I shows ISH-detected GFP-WRPE expression in liver biopsies, viewed at 20× magnification. FIG. 8J shows ISH-detected hFIX in liver biopsies, viewed at 20× magnification. FIG. 8K shows overlay image of ISH-detected GFP-WRPE and hFIX, viewed at 20× magnification. FIG. 8L shows ISH-detected GFP-WRPE and hFIX as an overlayed image with DAPI (staining for nuclei), viewed at 20× magnification. FIG. 8M shows ISH-detected GFP-WRPE and hFIX as an overlayed image with DAPI (staining for nuclei), viewed at 20× magnification in an untreated control.

FIG. 9 shows ARCUS-mediated on-target editing in NHP treated with AAVhu37.ARCUS2 and AAVhu37. Donor-HDR-hFIX. At 84 days post treatment, liver biopsies samples were collected, and percentage of total indels in the target region present in was calculated based on amplicon-seq.

FIGS. 10A and 10B show schematic representations of a PCSK9-hE7-KI Mouse Model. FIG. 10A shows schematic representation of the mouse pesk9 exon 7 which is replaced with human pcsk9 exon 7 (hE7 contains ARCUS targeting sequence). Human PCSK9 exon 7 sequence is shown in SEQ ID NO: 44. FIG. 10B shows schematic representation of crossing PCSK9-hE7-KI mouse model with other disease mouse models, such as OTC spf^{ash}, the KI-spf^{ash} model. The PCSK9-hE7-KI knock-in mouse model was first generated by replacing a region including exon 7 of the murine Pesk9 gene with a region of human PCSK9 gene containing exon 7. The PCSK9-hE7-KI mouse was then crossed with sparse fur ash (spf^{ash}) mouse, which exhibits a 20-fold reduction in OTC expression due to a G to A point mutation at the splice donor site at the end of exon 4 of the Ote gene. The mice from this cross were termed PCSK9-hE7-KI.spf^{ash} mice and were utilized as described herein. Abbreviations: bp, base pairs: E6, exon 6: E7: exon 7: E8, exon 8: HDR, homology-dependent recombination: PCSK9, proprotein convertase subtilisin/kexin type 9 (gene, human); Pesk9, proprotein convertase subtilisin/kexin type 9 (gene, mouse).

FIGS. 11A-11I show an in vivo test of nuclease-mediated gene targeting in newborn NHPs for vectors as shown in FIG. 11I. FIG. 11A is a chart showing experimental design of in vivo test of nuclease-mediated gene targeting in newborn NHPs for vectors as described in Example 3. Animals 21-111, 21-122, and 21-113 were AAV binding antibody (BAb) positive prior to dosing. Day 0 sample of 21-178 was collected post vector dosing which would interfere with the Bab assay. C: number of OT sites identified in independent ITRseq assay are listed. FIG. 11B shows PCSK9 levels shown as ng/ml (top row) or % of day 0 (bottom row) for the groups as shown in FIG. 11A. FIG. 11C shows ALT levels shown as U/L (top row) or AST shown as U/L (bottom row) for the groups, as shown in FIG. 11A. FIG. 11D shows transduction efficiency of OTC transgene as quantified by ISH or IF, and plotted as percent hepatocytes transduced. FIG. 11E shows body weight of mice. FIG. 11F shows vector GCs in liver by quantitative PCR analysis at day 84. FIG. 11G shows expression of hOTC and nuclease in macaque liver at day 84 measured by quantitative PCR on total RNA isolated from the liver biopsy samples followed by reverse transcription and presented as relative expression levels normalized by GAPDH levels. FIG. 11H shows Indel analysis on the rhPCSK9-targeted locus performed by amplicon-seq. FIG. 11I is a schematic of a timeline of an in vivo test of nuclease-mediated gene targeting in newborn NHPs including vectors tested for experiment described in Example 3.

FIG. 12 shows sequence alignment of 265 bp sequence that represents the human PCSK9 sequence of the pcsk9-hE7 knock-in allele, mouse PCSK9 (mPCSK9) and rhesus macaques PCSK9 (rhPCSK9). Abbreviations: GAPDH, glyceraldehyde-3-phosphate dehydrogenase: GC, genome copies: hOTC, human ornithine transcarbamylase: OT, off-target: PCR, polymerase chain reaction; rhPCSK9, proprotein convertase subtilisin/kexin type 9 (rhesus gene); RNA, ribonucleic acid, rhesus Exon 7—SEQ ID NO: 13.

FIG. 13 shows a schematic representation of donor constructs for a dual AAV vector system for ARCUS2-mediated gene correction, wherein the AAV-donor vector comprises an hOTC donor template sequence. The homology of the HDR arms in the constructs with the knock in mouse model (FIG. 10A-10B), NHP, and human target regions is shown.

FIG. 14A shows a timeline for a study comprising an hOTC mini-gene knock-in in PCSK9 locus by ARCUS2 performed in PCSK9-hE7-KI.spf-ash pups (partial OTC deficiency model), as described in Example 5.

FIG. 14B shows the vectors and dosages each group will receive for the study of FIG. 14A.

FIGS. 14C-14I show results of a study of mice treated with vectors as shown in FIG. 7, or untreated (KI WT) and fed a high protein (HP) diet for 10 days. FIG. 14C shows probability of survival. FIG. 14D shows weight as a percentage of weight prior to introduction of the HP diet. FIG. 14E shows plasma NH₃ levels at day 10 of HP diet. FIG. 14F shows mPCSK9 protein levels at day 48. FIG. 14G shows indel % as measured by amplicon-seq on day 59. FIG. 14H shows vector transduction levels in liver biopsy samples, plotted as AAV genome copies (GC) per diploid cell, measured on day 59. FIG. 14I shows OTC IF at 8 weeks.

FIG. 15 is schematic of the experimental design described in Example 10 to generate hLDLR mini gene knock-in in PCSK9 locus by SaCas9 in PCSK9-hE7-KI.ldlr-/ldlr-.apobec-/apobec-Pups (hoFH model).

FIG. 16 is a schematic showing the vectors used in Example 10.

FIG. 17 shows the experimental design of Example 10.

FIG. 18A-18D shows the results of the experiment of Example 10. FIG. 18A shows serum LDL levels for shHDR+saCas9, mhHDR+saCas9, shHDR only and untreated mice. FIG. 18B shows indel percentages for shHDR+saCas9, mhHDR+saCas9, shHDR only treated mice. FIG. 18C shows hLDLR genome copies per diploid genome as measured in liver at day 63. FIG. 18D shows serum LDL levels at day 63 for shHDR+saCas9, mhHDR+saCas9, shHDR only and untreated mice.

FIG. 19 shows immunohistochemistry data for liver samples taken at day 63 for mice of Example 10.

FIG. 20 shows an experimental timeline for a study of meganuclease-mediated targeted gene insertion in adolescent rhesus macaques.

FIG. 21A shows the vectors and dosages administered for G1 and G2 groups.

FIG. 21B shows plasma hFIX levels in the adolescent macaques (2.8 years old) at the indicated number of days post injection.

FIG. 22 shows ALT, AST, Bilirubin, APTT, PT and Platelet levels in the adolescent macaques at the indicated number of days post injection.

FIG. 23 shows absolute PCSK9 levels and as a percentage of day 0, and LDL levels as a percentage of day 0, at the indicated number of days and months post injection.

FIG. 24 shows an overview of animals and results for a study to assess the effects of ARCUS-mediated knock-in of the human FIX gene in macaques of different ages. *=animal euthanized at 1 yr. Values represent averages of all liver lobes at necropsy.

FIG. 25A and FIG. 25B show hFIX expression levels in animals of different ages. hFIX levels were above normal in both the 1.1 year and 2.8 year cohorts. Animal BO39 showed an anti-hFIX IgG response and was treated with prednisolone from days 48-84.

FIG. 26 shows ALT levels in animals of the indicated ages.

FIG. 27A shows platelet levels for animals BO39, BO44, and BO41.

FIG. 27B shows APTT levels for animals BO39, BO44, and BO41.

FIG. 28 shows results from evaluation of vector DNA and transgene/nuclease RNA expression.

FIG. 29 shows a schematic of the germ-line modification of exon 7 of the endogenous Pesk9 gene in the mouse OTC model.

FIG. 30A shows survival of untreated OTC model mice and OTC model mice treated with M2PCSK9 meganuclease (MN) vector alone or in combination with a mouse OTC donor vectors. #denotes significant differences in survival compared to the untreated control group (Pairwise-Log Rank analysis, p<0.05)

FIG. 30B shows changes in body weight percentages of untreated OTC model mice and OTC model mice treated with M2PCSK9 meganuclease (MN) vector alone or in combination with a mouse OTC donor vector. Values presented as mean±SEM. # denotes significant differences in body weight compared to the untreated control group (linear mixed model was fit and pairwise comparisons performed with the Tukey method used for p-value adjustment, p<0.05).

FIG. 31A-FIG. 31B show quantification of hepatocytes positive for in situ hybridization (ISH) signal (FIG. 31A) and IF signal (FIG. 31B) after 60 days in untreated OTC model mice and OTC model mice treated with M2PCSK9 meganuclease vector alone or in combination a mouse OTC donor vector. Values presented as mean±SEM. * denotes significant differences to other treatment groups (Kruskal-Wallis test followed by a Conover-Iman post-test to analyze pairwise comparisons, p<0.05).

FIGS. 32A-FIG. 32B show quantification of OTC enzyme activity (FIG. 32A) and levels of on-target genome editing calculated as the indel % (FIG. 32B) after 60 days in untreated OTC model mice and OTC model mice treated with an M2PCSK9 meganuclease vector alone or in combination with a mouse OTC donor vector.

FIG. 33 shows an experimental design of an NHP study to assess co-administration of Rituximab (Rtx) and/or M281 with an M2PCSK9 meganuclease vector alone or in combination with a mouse OTC donor vector.

FIG. 34A and FIG. 34B show rhIgG (FIG. 34A) and hFIX (FIG. 34B) expression levels following co-administration of an M2PCSK9 meganuclease vector and a mouse OTC donor vector.

### DETAILED DESCRIPTION OF THE INVENTION

Provided herein are compositions, kits, and methods which provide stable, long term therapeutic effects to patients with certain genetic disorders, including liver metabolic disorders. The compositions, kits, and methods utilize a nuclease that targets the PCSK9 locus of the target cell, and a donor vector provides a template which includes an exogenous product for integration into, and expression from, the PCSK9 locus, wherein the inserted nucleic acid sequence does not encode PCSK9, and the expression of the endogenous PCSK9 is disrupted and expression levels are reduced.

### PCSK9

Proprotein convertase subtilisin kexin 9 (PCSK9) is a serine protease that reduces both hepatic and extrahepatic low-density lipoprotein (LDL) receptor (LDLR: 606945) levels and increases plasma LDL cholesterol. PCSK9 is critical in the regulation of plasma cholesterol homeostasis. PCSK9 binds to the low-density lipid receptor family members low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments. Human PCSK9 has a protein sequence of NP_777596.2, as shown in SEQ ID NO: 23, with the coding sequence shown in SEQ ID NO: 22.

While the PCSK9 gene has been targeted for treatment of cholesterol related diseases, it is demonstrated herein that the PSCK9 gene locus is a safe harbor for gene targeting for insertion of other, non-PCSK9 transgenes. Thus, the compositions, kits, and methods provided herein utilize nucleases which target the PCSK9 gene locus, and insert a therapeutic transgene into the target PCSK9 locus, using a donor template.

The compositions, kits, and methods provided herein include a gene editing component (in some embodiments, a vector), and a donor vector which provides the therapeutic transgene to be expressed in the host cell.

### Gene Editing Component

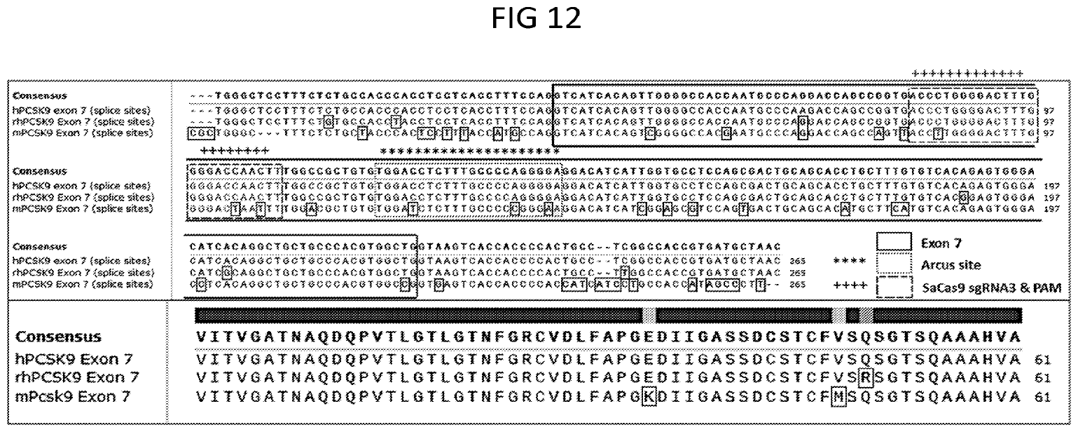

The compositions, kits, and methods provided herein include a gene editing component that comprises a nuclease (or the coding sequence therefore) and sequences which direct the nuclease to specifically target the native PCSK9 gene locus on chromosome 1. As used herein, the “target PCSK9 locus” or “PCSK9 gene locus” is any site in the PCSK9 coding region where insertion of the heterologous transgene is desired. In certain embodiments, the target PCSK9 locus is in Exon 7 of the PCSK9 coding sequence. FIG. 12 provides an alignment of the human (h), rhesus (rh), and mouse (m) PCSK9 exon 7 splice sites which are exemplified herein using a SaCas9 and a meganuclease targeted to PCSK9 (referred to as ARCUS).

Described herein are compositions, particularly nucleases, which are useful for targeting a gene for the insertion of a transgene, for example, nucleases that are specific for PCSK9. In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases: TAL-effector nucleases: meganuclease DNA-binding domains with heterologous cleavage domains).

In certain embodiments, the nuclease is a meganuclease that targets PCSK9. Meganucleases are endodeoxyribonucleases characterized by a large recognition site (double-stranded DNA sequences of 12 to 40 base pairs), for example, I-Scel. When combined with a nuclease, DNA can be cut at a specific location. The restriction enzymes can be introduced into cells, for use in gene editing or for genome editing in situ. In certain embodiments, the nuclease is a member of the LAGLIDADG (SEQ ID NO: 31) family of homing endonucleases. In certain embodiments, the nuclease is a member of the I-Crel family of homing endonucleases which recognizes and cuts a 22 base pair recognition sequence SEQ ID NO: 32-CAAAACGTCGTGAGACAGTTTG. See, e.g., WO 2009/059195. Methods for rationally-designing mono-LAGLIDADG (SEQ ID NO: 31) homing endonucleases were described which are capable of comprehensively redesigning I-Crel and other homing endonucleases to target widely-divergent DNA sites, including sites in mammalian, yeast, plant, bacterial, and viral genomes (WO 2007/047859). In one embodiment, the nuclease is encoded by the sequence shown in SEQ ID NO: 19, nt 330 to 1424, or a sequence sharing at least 95%, 98%, or 99% identity thereto. In one embodiment, the nuclease protein sequence is the sequence shown in SEQ ID NO: 20, or a sequence sharing at least 95%, 98%, or 99% identity thereto. Such nuclease is sometimes referred to herein as the ARCUS nuclease. The term “homing endonuclease” is synonymous with the term “meganuclease.” See, WO 2018/195449, describing certain PCSK9 meganucleases, which is incorporated herein in its entirety.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms and serve as a prominent tool in the field of genome editing. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). In another embodiment, the coding sequence encodes a zinc finger nuclease or a transcription activator-like (TAL) effector nuclease (TALEN).

In certain embodiments, the nuclease is a CRISPR-associated nuclease (Cas), optionally, Cas9. “Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Streptococcus pyogenes (SpCas9), and Neisseria meningitides [KM Estelt et al, Nat Meth, 10:1116-1121 (2013)]. The wild-type coding sequences may be utilized in the constructs described herein. Alternatively, the bacterial codons are optimized for expression in humans, e.g., using any of a variety of known human codon optimizing algorithms. Alternatively, these sequences may be produced synthetically, either in full or in part. Other endonucleases with similar properties may optionally be substituted. See, e.g., the public CRISPR database (db) accessible at http://crispr.u-psud.fr/crispr.

In certain embodiments, the compositions, kits, and methods the nuclease coding sequence is comprised in a gene editing vector. The gene editing vector includes an expression cassette comprising a nucleic acid sequence encoding a nuclease and regulatory sequences that direct expression of the nuclease in a target cell comprising a PCSK9 gene.

A “vector” as used herein is a biological or chemical moiety comprising a nucleic acid sequence which can be introduced into an appropriate host cell for replication or expression of said nucleic acid sequence. Common vectors include non-viral vectors and viral vectors. As used herein, a non-viral system might be selected from nanoparticles, electroporation systems and novel biomaterials, naked DNA, phage, transposon, plasmids, cosmids (Phillip McClean, www.ndsu.edu/pubweb/˜mcclean/-plsc731/cloning/cloning4.htm) and artificial chromosomes (Gong, Shiaoching, et al. “A gene expression atlas of the central nervous system based on bacterial artificial chromosomes.” Nature 425.6961 (2003): 917-925).

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous with the nucleic acid sequence and regulatory sequences that act in trans or at a distance to control the sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region comprising a polyadenylation site, among other elements. In other embodiments, the term “transgene” refers to one or more DNA sequences from an exogenous source which are inserted into a target cell. Typically, such an expression cassette for generating a viral vector contains the coding sequence for the gene product described herein flanked by packaging signals of the viral genome and other expression control sequences such as those described herein. In certain embodiments, a vector genome may contain two or more expression cassettes.

In addition to the coding sequence for the nuclease, in certain embodiments the gene editing vector includes regulatory sequences which direct expression of the nuclease in a host cell. In certain embodiments, the regulatory elements include a promoter. In certain embodiments, where the system is designed for treatment of metabolic disorders characterized by a mutation or phenotype in hepatocytes, the gene editing vector may be designed such that the nuclease is expressed under the control of a liver-specific promoter. An illustrative plasmid and vector described herein uses the liver-specific promoter thyroxin binding globulin (TBG), which is characterized by the sequence of SEQ ID NO: 41. In other embodiments a shortened version of TBG, a variant termed herein TBG-S1, which is characterized by the sequence of SEQ ID NO: 11, is useful. In another embodiment, the hybrid liver promoter (HLP) having the sequence of SEQ ID NO: 12 is utilized.

In some embodiments it is desirable to utilize a promoter having low-transcriptional activity, or weak promoter. In one embodiment, the promoter is a weakened version of the liver-specific thyroxin binding globulin (TBG) promoter. In one embodiment, the weak promoter is truncated at the 5′ or 3′ end of the native promoter, or TBG-SI sequence. In another embodiment, the promoter retains only the 3′ terminal 113 nt from the TBG-SI promoter and is termed F113 (also called TBG-S1-F113) (SEQ ID NO: 19, nt 206 to 318). U.S. Provisional Patent Application Nos. 63/016,145, filed Apr. 27, 2020, 63/033,738, filed Jun. 2, 2020, and 63/089,796, filed Oct. 9, 2020, PCT/US21/29386 and PCT/US21/29403, both filed Apr. 27, 2021, each entitled COMPOSITIONS AND METHODS FOR REDUCING NUCLEASE EXPRESSION AND OFF-TARGET ACTIVITY USING A PROMOTER WITH LOW TRANSCRIPTIONAL ACTIVITY are incorporated herein by reference in their entirety.

Alternatively, other liver-specific promoters may be used such as alpha 1 anti-trypsin (A1AT), human albumin (Miyatake et al., J. Virol., 71:5124 32 (1997)), and hepatitis B virus core promoter (Sandig et al., Gene Ther., 3:1002 9 (1996), TTR minimal enhancer/promoter, alpha-antitrypsin promoter, LSP (845 nt). See, e.g., The Liver Specific Gene Promoter Database, Cold Spring Harbor, http://rulai.schl.edu/LSPD. Alternatively, other tissue specific promoters, such as muscle-specific promoters, such as the muscle creatine kinase (MCK) promoter, or muscle hybrid (MH) promoter, may be used. Alternatively, other promoters, such as constitutive promoters (CMV, CBG, CB7, etc.), regulatable (inducible) promoters [see, e.g., WO 2011/126808 and WO 2013/049493, incorporated by reference herein], or a promoter responsive to physiologic cues may be utilized in the vectors described herein. Optionally, if a regulatable system is selected, a third vector may be required in order to provide the regulatory function.

In addition to a promoter, the gene editing cassette, expression cassette and/or vector may contain one or more appropriate “regulatory elements” or “regulatory sequences”, which comprise but are not limited to an enhancer: transcription factor: transcription terminator; efficient RNA processing signals such as splicing and polyadenylation signals (polyA): sequences that stabilize cytoplasmic mRNA, for example Woodchuck Hepatitis Virus (WHV) Posttranscriptional Regulatory Element (WPRE): sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequence or transgene coding sequence.

In certain embodiments, the gene editing vector includes a TBG promoter, one or more alpha mic/bik enhancer(s), coding sequence for the ARCUS meganuclease, optionally a WPRE, and a polyA. In certain embodiments, the expression cassette includes nt 211 to nt 2964 of SEQ ID NO: 42.

In some embodiments, the gene editing component further includes sequences which direct the nuclease to a target site in the PCSK9 target locus. In certain embodiments, such as a meganuclease specific for PCSK9, no further sequences are required to direct the nuclease to the target site. However, in the case, for example, of Cas9, an additional sequence, called a “single guide RNA” or “sgRNA” is provided, which is specific for the target sequence. The sgRNA may be provided on the same vector (cis) or a different vector from (trans) as the Cas9. As used herein, the sgRNA has at least a 20-base sequence (or about 24-28 bases, sometimes called the seed region) for specific DNA binding (i.e., homologous to the target DNA), in combination with the gRNA scaffold. Transcription of sgRNAs should start precisely at its 5′ end. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence. Optionally, the gene editing vector may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. In one embodiment, the sgRNA “seed” coding sequence is AAGTTGGTCCCCAAAGTCCC (SEQ ID NO: 8), which is useful for targeting exon 7 of human and macaque PCSK9 by SaCas9. However, other sgRNAs can be designed by the person of skill in the art.

In certain embodiments, the sgRNA includes at least 20 nucleotides and specifically binds to a target site in the PCSK9 gene, said target site being 5′ to a protospacer-adjacent motif (PAM) that is specifically recognized by the Cas9. The seed region in some embodiments shares 100% complementarity with the target site in the PCSK9 gene. In other embodiments, the seed region contains 1, 2, 3, 4, or 5 mismatches as compared to the target site.

The sgRNA is under control of an RNA polymerase promoter and/or terminator. In certain embodiments, the RNA polymerase promoter is a Pol III promoter such as the U6 promoter. In another embodiment, the promoter is the H1 promoter. The sequence for an exemplary U6 promoter can be found in SEQ ID NO: 10. In other embodiments, the sgRNA and RNA polymerase promoter are located in the donor vector.

In other embodiments, for example, wherein the nuclease is a Cas9, the gene editing component further includes one or more nuclear localization signal (NLSs). In one embodiment, the NLSs flank the coding sequence for the Cas9. In certain embodiments, the NLS has the sequence of nt 4241 to 4288 of SEQ ID NO: 5. See, e.g., Lu et al. Types of nuclear localization signals and mechanisms of protein import into the nucleus, Cell Commun Signal (May 2021) 19:60, which is incorporated herein by reference.

In certain embodiments, the nuclease coding sequence is provided as messenger RNA (mRNA). An mRNA may include a 5′ untranslated region, a 3′ untranslated region, and/or a coding or translating sequence. In certain embodiments, the coding sequence for a Cas9 is provided as mRNA.

An mRNA may be a naturally or non-naturally occurring mRNA. An mRNA may include one or more modified nucleobases, nucleosides, or nucleotides. In some embodiments, the mRNA in the compositions of the invention comprise at least one modification which confers increased or enhanced stability to the nucleic acid, including, for example, improved resistance to nuclease digestion in vivo. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g. the wild-type version of the mRNA). Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozak consensus sequence).

In some embodiments, the mRNA described herein have undergone a chemical or biological modification to render them more stable. Exemplary modifications to an mRNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring mRNA, for example, covalent modifications such as the introduction of modified nucleotides, (e.g., nucleotide analogs, or the inclusion of pendant groups which are not naturally found in such mRNA molecules).

In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids of the present invention also include the incorporation of pseudouridines pseudouridine (ψ) or 5-methylcytosine (m5C). Substitutions and modifications to the mRNA of the present invention may be performed by methods readily known to one or ordinary skill in the art.

In certain embodiments, the mRNA includes a 5′ cap structure, a chain terminating nucleotide, a stem loop, and/or a polyadenylation signal. A cap structure or cap species is a compound including two nucleoside moieties joined by a linker and may be selected from a naturally occurring cap, a non-naturally occurring cap or cap analog, or an anti-reverse cap analog. An mRNA may instead or additionally include a chain terminating nucleoside.

In certain embodiments, the mRNA includes a stem loop, such as a histone stem loop. A stem loop may include 1, 2, 3, 4, 5, 6, 7, 8, or more nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

In certain embodiments, the mRNA includes a polyA sequence. A polyA sequence may be comprised entirely or mostly of adenine nucleotides or analogs or derivatives thereof. In certain embodiments, the polyA sequence is a tail located adjacent to a 3′ untranslated region of an mRNA.

An mRNA may encode any polypeptide of interest, e.g., a nuclease, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

An exemplary gene editing vector genome encoding SaCas9 is shown in SEQ ID NO: 5. In another embodiment, an expression cassette is provided comprising nt 193-4502 of SEQ ID NO: 5.

### Donor Vector

The compositions, kits, and methods include a donor vector, which provides the coding sequence for the therapeutic transgene, i.e., Factor IX (FIX, F9). In certain embodiments, the donor vector contains an expression cassette comprising a nucleic acid sequence encoding a transgene, and regulatory sequences that direct expression of the transgene in the target cell. In certain embodiments, the transgene encodes a protein that is aberrantly expressed in a liver metabolic disorder or other genetic disorder. The transgene encodes a protein other than PCSK9. In certain embodiments, the transgene encodes Factor IX. Factor IX is a vitamin K-dependent plasma protein that participates in the intrinsic pathway of blood coagulation by converting factor X to its active form in the presence of Ca2+ ions, phospholipids, and factor VIIIa. Hemophilia B is an X-linked blood coagulation disorder characterized by a permanent tendency to hemorrhage, due to factor IX deficiency. It is phenotypically similar to hemophilia A, but patients present with fewer symptoms.

Many patients are asymptomatic until the hemostatic system is stressed by surgery or trauma. The sequence of Factor IX is known in the art, and is shown, e.g., at Uniprot accession no. P00740.

In certain embodiments, Factor IX includes proteins such as the sequence shown in SEQ ID NO: 56, or a sequence sharing at least 70%, 75%, 80%, 85%, 90%, 91%, 92%, 93%, 94%, 95%, 96%, 97%, 98%, 99% identity with SEQ ID NO: 56. In certain embodiments, the Factor IX is encoded by the sequence shown in SEQ ID NO: 55, or a sequence sharing at least 70%, 75%, 80%, 85%, 90%, 91%, 92%, 93%, 94%, 95%, 96%, 97%, 98%, 99% identity with SEQ ID NO: 55.

In certain embodiments, Factor IX includes proteins such as the sequence shown in SEQ ID NO: 82, or a sequence sharing at least 70%, 75%, 80%, 85%, 90%, 91%, 92%, 93%, 94%, 95%, 96%, 97%, 98%, 99% identity with SEQ ID NO: 82. In certain embodiments, the Factor IX is encoded by the sequence shown in SEQ ID NO: 81, or a sequence sharing at least 70%, 75%, 80%, 85%, 90%, 91%, 92%, 93%, 94%, 95%, 96%, 97%, 98%, 99% identity with SEQ ID NO: 81.

Other Factor IX proteins useful herein include active variants, such as those described in US 2019/0017039A1, US 2021/0330763A1, US 2019/0192640A1, US 2019/0240350A1, and US 2021/0230570A1, each of which is incorporated herein by reference. In certain embodiments, Factor IX-Padua is used, in which a R384L mutation is present, using the sequence of SEQ ID NO: 82. See, e.g., Samelson-Jones B J, Finn J D, George L A, Camire R M, Arruda V R. Hyperactivity of factor IX Padua (R338L) depends on factor VIIIa cofactor activity. JCI Insight. 2019 Jun. 20: 5 (14): e128683, which is incorporated herein by reference.

In some embodiments, the transgene cassette includes a promoter, the transgene coding sequence, and a poly A sequence. In some embodiments, the promoter is a liver-specific promoter, such as the TBG promoter, TBG-SI promoter, HLP promoter, or others described herein. In other embodiments, a transgene is provided without a promoter, and is inserted in the genome downstream of the native PSCK9 promoter.

The transgene cassette, expression cassette and/or vector (editing or donor) may contain one or more appropriate “regulatory elements” or “regulatory sequences”, which comprise but are not limited to an enhancer: transcription factor: transcription terminator; efficient RNA processing signals such as splicing and polyadenylation signals (polyA); sequences that stabilize cytoplasmic mRNA, for example Woodchuck Hepatitis Virus (WHV) Posttranscriptional Regulatory Element (WPRE); sequences that enhance translation efficiency (i.e., Kozak consensus sequence); sequences that enhance protein stability; and when desired, sequences that enhance secretion of the encoded product. Examples of suitable polyA sequences include, e.g., SV40, bovine growth hormone (bGH), and TK polyA. Examples of suitable enhancers include, e.g., the alpha fetoprotein enhancer, the TTR minimal promoter/enhancer, LSP (TH-binding globulin promoter/alpha1-microglobulin/bikunin enhancer), amongst others. These control sequences or the regulatory sequences are operably linked to the nuclease coding sequences or transgene coding sequence.

In addition to the transgene cassette, in certain embodiments, the donor vector also includes homology-directed recombination (HDR) arms 5′ and 3′ to the transgene cassette, to facilitate homology directed recombination of the transgene into the endogenous genome. The homology arms are directed to the target PCSK9 locus and can be of varying length. In some embodiments, the HDR arms are each from about 100 bp to about 1000 bp in length. In other embodiments, the HDR arms are each from about 130 bp to about 500 bp. In other embodiments, the HDR arms are each from about 100 bp to about 300 bp. In other embodiments, the HDR arms are each from about 100 bp to about 400 bp. In other embodiments, the HDR arms are each from about 250 bp to about 500 bp. In other embodiments, the HDR arms are each from about 300 bp to about 500 bp. In certain embodiments, the HDR arms are each about 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 450 bp, 475 bp, or 500 bp. In one embodiment, the HDR arm is 130 bp. In another embodiment, the HDR arm is 137 bp. In other embodiments, the HDR arms are about 130 bp to 140 bp. In another embodiment, the HDR arms are about 500 bp. In another embodiment, the HDR arms are absent. The HDR arms ideally share a high level of complementarity with the target PCSK9 locus, although it need not be 100% complementarity. In some embodiments, 1, 2, 3, 4, 5, 6, 7, 8, 9, 10, 11, 12, 13, 14, 15, 16, 17, 18, 19, 20, or more mismatches are permitted in each HDR arm. Suitable HDR arm sequences, for targeting PCSK9 exon 7 are shown in SEQ ID Nos: 24-29. In one embodiment, the HDR arm sequences are selected from SEQ ID Nos: 24-29.

Also provided herein, are compositions, kits, and methods for nuclease-mediated, site-specific integration of a Factor IX transgene cassette in a PCSK9 safe harbor in the genome that provides long-term therapeutic benefits to patients with hemophilia.

### Viral and Non-Viral Vectors

The (gene editing and donor) expression cassettes or coding sequences described herein, may be engineered into any suitable genetic element for delivery to a target cell, e.g., a liver cell, such as a vector. In one embodiment, a nucleic acid is delivered via non-viral vector or lipid nanoparticle, as described herein or known in the art.

In certain embodiments, the gene editing component is encapsulated in a lipid nanoparticle (LNP). See, for example, Conway et al, Non-viral Delivery of Zinc Finger Nuclease mRNA Enables Highly Efficient In Vivo Genome Editing of Multiple Therapeutic Gene Targets, Molecular Therapy, 27 (4): 866-877 (April 2019), which is incorporated herein by reference). As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more mRNA to one or more target cells (e.g., liver and/or muscle). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a mRNA to a target cell. Useful lipid nanoparticles for mRNA comprise a cationic lipid to encapsulate and/or enhance the delivery of mRNA into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid. PEG-lipid and polymer forming a lipid bilayer around the encapsulated nucleic acids (Kowalski et al., 2019, Mol. Ther. 27 (4): 710-728). In some embodiments, LNP comprises a cationic lipid (i.e. N-[1-(2,3-dioleoyloxy) propyl]-N, N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly (β-amino) esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference. In certain embodiments, wherein the gene editing component includes a Cas9 mRNA, the LNP also includes a gRNA.

Certain LNPs useful herein include those that are described in WO 2021/077066, WO 2021/055892, and PCT/US23/65720 each of which is incorporated herein by reference in its entirety. Useful LNPs include those that show enhanced delivery to the liver. LNP formulations may be varied to enhance liver delivery. For example, the type and ionizable lipid: mRNA ratio, the mRNA: sgRNA ratio, molar ratio of ionizable lipid, phosopholipid, cholesterol, and PEG-lipid, etc. may be varied. In one embodiment, the LNP is one described by Kauffman, K. J.: Dorkin, J. R.; Yang, J. H.; Heartlein, M. W.: DeRosa, F.; Mir, F. F.: Fenton, O. S.: Anderson, D. G., Optimization of lipid nanoparticle formulations for mRNA delivery in vivo with fractional factorial and definitive screening designs. Nano letters 2015, 15 (11), 7300-7306, which is incorporated herein by reference. In certain embodiments, the LNPs are designed with ionizable lipid: mRNA weight ratios varying between 5:1 to 25:1. In certain embodiments, the ionizable lipid: mRNA weight ratio is 1:1, 2:1, 3:1, 4:1, 5:1, 6:1, 7:1, 8:1, 9:1, 10:1, 12.5:1, 15:1, 20:1, or 25:1. In certain embodiments, the mRNA: sgRNA weight ratio is 1:1, 1:2, 2:1, 1:4, 1:5, 5:1, 4:1, 3:1, or 2:1.

Other LNPs have been described and are useful herein. See, e.g., WO 2016/118724. U.S. Pat. No. 10,413,618B2, U.S. Pat. No. 10,723,692B2, and U.S. Pat. No. 8,754,062B2, each of which is incorporated herein by reference.

In certain embodiments, the gene editing component, e.g., Cas9, is provided in an LNP.

Certain examples herein illustrate use of AAV vectors containing the gene editing component (nuclease) coding sequences and transgene coding sequences in AAV vector genomes. However, the use of constructs described herein is not limited to AAV constructs and can be used for other vectors. In certain embodiments, the vector genome may be packaged into a different vector (e.g., a recombinant bocavirus). In certain embodiments, the expression cassette may be packaged into a different viral vector, into a non-viral vector, and/or into a different delivery system. In certain embodiments, the gene editing component is provided in an LNP.

“Plasmid” or “plasmid vector” generally is designated herein by a lowercase p preceded and/or followed by a vector name. Plasmids, other cloning and expression vectors, properties thereof, and constructing/manipulating methods thereof that can be used in accordance with the present invention are readily apparent to those of skill in the art. In one embodiment, the nucleic acid sequence as described herein or the expression cassette as described herein are engineered into a suitable genetic element (a vector) useful for generating viral vectors and/or for delivery to a host cell, e.g., naked DNA, phage, transposon, cosmid, episome, etc., which transfers the nuclease sequences carried thereon. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY.

In certain embodiments, the expression cassette is located in a vector genome for packaging into a viral capsid. For example, for an AAV vector genome, the components of the expression cassette are flanked at the extreme 5′ end and the extreme 3′ end by AAV inverted terminal repeat sequences. For example, a 5′ AAV ITR, expression cassette, 3′ AAV ITR. In other embodiments, a self-complementary AAV may be selected. In other embodiments, retroviral system, lentivirus vector system, or an adenoviral system may be used.

### AAV Vectors

In certain embodiments, the gene editing vector and/or the donor vector is provided as a recombinant AAV. A “recombinant AAV” or “rAAV” is a DNAse-resistant viral particle containing two elements, an AAV capsid and a vector genome containing at least non-AAV coding sequence packaged within the AAV capsid. Unless otherwise specified, this term may be used interchangeably with the phrase “rAAV vector” or “AAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The source of the AAV capsid may be one of any of the dozens of naturally occurring and available adeno-associated viruses, as well as engineered AAVs. The source of the AAV capsid for the gene editing vector and/or the donor vector is, in one embodiment, the same. In another embodiment, the source of the AAV capsid for the gene editing vector and/or the donor vector is different. An adeno-associated virus (AAV) viral vector is an AAV DNase-resistant particle having an AAV protein capsid into which is packaged nucleic acid sequences for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Published Patent Application No. 2007-0036760-A1: US Published Patent Application No. 2009-0197338-A1: EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, WO 2003/042397 (rh.10) and WO 2018/160582 (AAVhu68). These documents also describe other AAV which may be selected for generating AAV and are incorporated by reference.

Unless otherwise specified, the AAV capsid. ITRs, and other selected AAV components described herein, may be readily selected from among any AAV, including, without limitation, the AA Vs commonly identified as AAV1, AAV2, AAV3, AAV4, AAV5, AAV6, AAV7, AAV8, AAV9, AAV8 bp, AAV7M8, AAVAnc80, AAVrh10, AAVrh79, and AAVPHP.B and variants of any of the known or mentioned AAVs or AAVs yet to be discovered or variants or mixtures thereof. See, e.g., WO 2005/033321, which is incorporated herein by reference. In one embodiment, the AAV capsid is an AAV1 capsid or variant thereof, AAV8 capsid or variant thereof, an AAV9 capsid or variant thereof, an AAVhu.68 capsid or variant thereof, an AAVrh.10 capsid or variant thereof, an AAVrh64R1 capsid or variant thereof, an AAVhu.37 capsid or variant thereof, or an AAV3B or variant thereof. In one aspect, the capsid is an AAVhu.37 capsid. See, also WO 2019/168961 and WO 2019/169004, which are incorporated by reference herein in their entirety. In other embodiments, the AAV capsid is an AAVrh79 capsid or variant thereof. In other embodiments, the AAV capsid is an AAVrh.90 or variant thereof.

In certain embodiments, the rAAV comprises an AAVhu37 capsid. An AAVhu37 capsid comprises: a heterogeneous population of vp1 proteins which are the product of a nucleic acid sequence encoding the amino acid sequence of SEQ ID NO: 38, a heterogeneous population of vp2 proteins which are the product of a nucleic acid sequence encoding the amino acid sequence of at least about amino acids 138 to 738 of SEQ ID NO: 38, and a heterogeneous population of vp3 proteins which are the product of a nucleic acid sequence encoding at least amino acids 204 to 738 of SEQ ID NO: 38 wherein: the vp1, vp2 and vp3 proteins contain subpopulations with amino acid modifications comprising at least two highly deamidated asparagines (N) in asparagine-glycine pairs in SEQ ID NO: 38 and optionally further comprising subpopulations comprising other deamidated amino acids, wherein the deamidation results in an amino acid change. AAVhu37 is characterized by having highly deamidated residues, e.g., at positions N57, N263, N385, and/or N514 based on the numbering of the AAVhu37 VP1 (SEQ ID NO: 38).

Deamidation has been observed in other residues, as shown in the table below, and in, e.g., WO 2019/168961, published Sep. 6, 2019, which is incorporated herein by reference. In certain embodiments, an AAVhu37 capsid is modified in one or more of the following positions, in the ranges provided below, as determined using mass spectrometry with a trypsin enzyme. In certain embodiments, one or more of the following positions, or the glycine following the N is modified as described herein. For example, in certain embodiments, a G may be modified to an S or an A, e.g., at position 58, 264, 386, or 515. In one embodiment, the AAVhu37 capsid is modified at position N57/G58 to N57Q or G58A to afford a capsid with reduced deamidation at this position. In another embodiment, N57/G58 is altered to NS57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location.

In certain embodiments, AAVhu37 may have these or other residues deamidated, e.g., typically at less than 10% and/or may have other modifications, including methylations (e.g. ˜R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g. at one or more of W248, W307, W307, M405, M437, M473, W480, W480, W505, M526, M544, M561, W621, M637, and/or W697). Optionally the W may oxidize to kynurenine.

TABLE A

| AAVhu37 | |
|---|---|
| Deamidation based | |
| on VP1 numbering | % Deamidation |
| N57 + Deamidation | 65-90, 70-95, 80- |
| | 95, 75-100, 80- |
| | 100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80- |
| | 95, 75-100, 80- |
| | 100, or 90-100 |
| ~N410 + Deamidation | 1-25, |
| N479 + Deamidation | 1-5, 1-3 |
| ~N514 + Deamidation | 65-90, 70-95, 80- |
| | 95, 75-100, 80- |
| | 100, or 90-100 |
| ~Q601 + Deamidation | 0-1 |
| N653 + Deamidation | 0-2 |

Still other positions may have such these or other modifications (e.g., acetylation or further deamidations). In certain embodiments, the nucleic acid sequence encoding the AAVhu37 vp1 capsid protein is provided in SEQ ID NO: 37. In other embodiments, a nucleic acid sequence of 70% to 99.9% identity to SEQ ID NO: 37 may be selected to express the AAVhu37 capsid proteins. In certain other embodiments, the nucleic acid sequence is at least about 75% identical, at least 80% identical, at least 85%, at least 90%, at least 95%, at least 97% identical, or at least 99% identical to SEQ ID NO: 37. However, other nucleic acid sequences which encode the amino acid sequence of SEQ ID NO: 38 may be selected for use in producing rAAVhu37 capsids. In certain embodiments, the nucleic acid sequence has the nucleic acid sequence of SEQ ID NO: 37 or a sequence at least 70% to at least 99% identical, at least 75%, at least 80%, at least 85%, at least 90%, at least 95%, at least 97%, at least 99%, identical to SEQ ID NO: 37 which encodes SEQ ID NO: 38. In certain embodiments, the nucleic acid sequence has the nucleic acid sequence of SEQ ID NO: 37 or a sequence at least 70% to 99%, at least 75%, at least 80%, at least 85%, at least 90%, at least 95%, at least 97%, at least 99%, identical to about nt 412 to about nt 2214 of SEQ ID NO: 37 which encodes the vp2 capsid protein (about aa 138 to 738) of SEQ ID NO: 38. In certain embodiments, the nucleic acid sequence has the nucleic acid sequence of about nt 610 to about nt 2214 of SEQ ID NO: 37 or a sequence at least 70% to 99%, at least 75%, at least 80%, at least 85%, at least 90%, at least 95%, at least 97%, at least 99%, identical to nt SEQ ID NO: 37 which encodes the vp3 capsid protein (about aa 204 to 738) of SEQ ID NO: 38. See, EP 2 345 731 B1 and SEQ ID NO: 88 therein, which are incorporated by reference.

In certain embodiments, the rAAV comprises an AAV8 capsid. An AAV8 capsid comprises: a heterogeneous population of VP isoforms which are deamidated as defined in the following table, based on the total amount of VP proteins in the capsid, as determined using mass spectrometry. Suitable modifications include those described in the paragraph above labelled modulation of deamidation, which is incorporated herein. In certain embodiments, the AAV capsid is modified at one or more of the following position, in the ranges provided below, as determined using mass spectrometry. In certain embodiments, one or more of the following positions, or the glycine following the N is modified as described herein. In certain embodiments, an artificial NG is introduced into a different position than one of the positions identified below. In certain embodiments, one or more of the following positions, or the glycine following the N is modified as described herein. For example, in certain embodiments, a G may be modified to an S or an A, e.g., at position 58, 67, 95, 216, 264, 386, 411, 460, 500, 515, or 541. Significant reduction in deamidation is observed when NG57/58 is altered to NS 57/58 or NA57/58. However, in certain embodiments, an increase in deamidation is observed when NG is altered to NS or NA. In certain embodiments, an N of an NG pair is modified to a Q while retaining the G. In certain embodiments, both amino acids of an NG pair are modified. In certain embodiments, N385Q results in significant reduction of deamidation in that location. In certain embodiments, N499Q results in significant increase of deamidation in that location. In certain embodiments, an NG mutation is made at the pair located at N263 (e.g., to N263A). In certain embodiments, an NG mutation is made at the pair located at N514 (e.g., to N514A). In certain embodiments, an NG mutation is made at the pair located at N540 (e.g., N540A). In certain embodiments, AAV mutants containing multiple mutations and at least one of the mutations at these positions are engineered. In certain embodiments, no mutation is made at position N57. In certain embodiments, no mutation is made at position N94. In certain embodiments, no mutation is made at position N305. In certain embodiments, no mutation is made at position G386. In certain embodiments, no mutation is made at position Q467. In certain embodiments, no mutation is made at position N479. In certain embodiments, no mutation is made at position N653. In certain embodiments, the capsid is modified to reduce “N” or “Q” at positions other than then “NG” pairs. Residue numbers are based on the published AAV8 sequence, reproduced in SEQ ID NO: 36.

TABLE B

| AAV8 Modification Based on | |
|---|---|
| VP1 numbering | % |
| N35 + Deamidation | 1 |
| N57 + Deamidation | 65-90, 70-95, 80-95, 75- |
| | 100, 80-100, or 90-100 |
| N66 + Deamidation | 0-10 |
| N94 + Deamidation | 1-15 |
| N113 + Deamidation | 0-10 |
| ~Q166 + Deamidation | 0-10 |
| ~N173 + Deamidation | 0-10 |
| N254/N255 + Deamidation | 5-45 |
| N263 + Deamidation | 65-90, 70-95, 80-95, 75- |
| | 100, 80-100, or 90-100 |
| ~N304 + Deamidation | 0-10 |
| ~N305 + Deamidation | 10-40 |
| N320 + Deamidation | 0-10 |
| ~Q322 + Deamidation | 0-10 |
| N385 + Deamidation | 65-90, 70-95, 80-95, 75- |
| | 100, 80-100, or 90-100 |
| N410 + Deamidation | 15-70 |
| ~Q431 + Deamidation | 0-10 |
| N438 + Deamidation | 0-10 |
| ~N459 + Deamidation | 0-10 |
| ~Q467 + Deamidation | 0-10 |
| ~N479 + Deamidation | 0-10 |
| N498/N499 + Deamidation | 0-10 |
| N502 + Deamidation | 0-10 |
| N514 + Deamidation | 65-90, 70-95, 80-95, 75- |
| | 100, 80-100, or 90-100 |
| N517 + Deamidation | 15-40 |
| N540 + Deamidation | 65-90, 70-95, 80-95, 75- |
| | 100, 80-100, or 90-100 |
| ~N554 + Deamidation | 0-10 |
| ~Q589 + Deamidation | 0-10 |
| ~N590 + Deamidation | 0-10 |
| ~N599 + Deamidation | 35-75 |
| ~Q601 + Deamidation | 45-75 |
| ~Q610 + Deamidation | 0-10 |
| Q617 + Deamidation | 0-10 |
| N630 + Deamidation | 5-30 |
| Q648 + Deamidation | 0-10 |
| N653 + Deamidation | 0-10 |
| N665 + Deamidation | 5-30 |
| N670 + Deamidation | 0-10 |
| N693 + Deamidation | 0-10 |
| ~N706 + Deamidation | 0-10 |
| N718 + Deamidation | 0-10 |
| N737 + Deamidation | 0-10 |

In certain embodiments, the rAAV comprises a AAVrh79 capsid, as described in WO 2019/169004, published Sep. 6, 2019, which is incorporated herein by reference. In one embodiment, an AAVrh79 capsid comprises a heterogeneous population of AAVrh79 vp1 proteins, AAVrh79 vp2 proteins, and AAVrh79 vp3 proteins. In one embodiment, the AAVrh79 capsid is produced by expression from a nucleic acid sequence which encodes the predicted amino acid sequence of 1 to 738 of SEQ ID NO: 34. Optionally, sequences co-expressing the vp3 protein from a nucleic acid sequence excluding the vp1-unique region (about aa 1 to 137) or the vp2-unique region (about aa 1 to 203), vp1 proteins produced from SEQ ID NO: 33, or vp1 proteins produced from a nucleic acid sequence at least 70% identical to SEQ ID NO: 33 which encodes the predicted amino acid sequence of 1 to 738 of SEQ ID NO: 34. In other embodiments, the AAVrh79 vp2 proteins produced by expression from a nucleic acid sequence which encodes the predicted amino acid sequence of at least about amino acids 138 to 738 of SEQ ID NO: 34, vp2 proteins produced from a sequence comprising at least nucleotides 412 to 2214 of SEQ ID NO: 33, or vp2 proteins produced from a nucleic acid sequence at least 70% identical to at least nucleotides 412 to 2214 of SEQ ID NO: 33 which encodes the predicted amino acid sequence of at least about amino acids 138 to 738 of SEQ ID NO: 34, AAVrh79 vp3 proteins produced by expression from a nucleic acid sequence which encodes the predicted amino acid sequence of at least about amino acids 204 to 738 of SEQ ID NO: 34, vp3 proteins produced from a sequence comprising at least nucleotides 610 to 2214 of SEQ ID NO: 33, or vp3 proteins produced from a nucleic acid sequence at least 70% identical to at least nucleotides 610 to 2214 of SEQ ID NO: 33 which encodes the predicted amino acid sequence of at least about amino acids 204 to 738 of SEQ ID NO: 34.

In certain embodiments, an AAVrh79 capsid comprises: a heterogeneous population of vp1 proteins which are the product of a nucleic acid sequence encoding the amino acid sequence of SEQ ID NO: 34, a heterogeneous population of vp2 proteins which are the product of a nucleic acid sequence encoding the amino acid sequence of at least about amino acids 138 to 738 of SEQ ID NO: 34, and a heterogeneous population of vp3 proteins which are the product of a nucleic acid sequence encoding at least amino acids 204 to 738 of SEQ ID NO: 34.

The AAVrh79 vp1, vp2 and vp3 proteins contain subpopulations with amino acid modifications comprising at least two highly deamidated asparagines (N) in asparagine-glycine pairs in SEQ ID NO: 34 and optionally further comprising subpopulations comprising other deamidated amino acids, wherein the deamidation results in an amino acid change. High levels of deamidation at N-G pairs N57, N263, N385 and/or N514 are observed, relative to the number of SEQ ID NO: 34. Deamidation has been observed in other residues, as shown in the table below and in the examples. In certain embodiments. AAVrh79 may have other residues deamidated, e.g., typically at less than 10% and/or may have other modifications, including methylations (e.g. ˜R487) (typically less than 5%, more typically less than 1% at a given residue), isomerization (e.g., at D97) (typically less than 5%, more typically less than 1% at a given residue, phosphorylation (e.g., where present, in the range of about 10 to about 60%, or about 10 to about 30%, or about 20 to about 60%) (e.g., at one or more of S149, ˜S153, ˜S474, ˜T570, ˜S665), or oxidation (e.g. at one or more of W248, W307, W307, M405, M437, M473, W480, W480, W505, M526, M544, M561, W621, M637, and/or W697). Optionally the W may oxidize to kynurenine.

TABLE C

| AAVrh79 | |
|---|---|
| Deamidation based | |
| on VP1 numbering | % Deamidation |
| N57 + Deamidation | 65-90, 70-95, 80- |
| | 95, 75-100, 80- |
| | 100, or 90-100 |
| N94 + Deamidation | 5-15, about 10 |
| ~N254 + Deamidation | 10-20 |
| ~N263 + Deamidation | 75-100 |
| ~N305 + Deamidation | 1-5 |
| ~N385 + Deamidation | 65-90, 70-95, 80- |
| | 95, 75-100, 80- |
| | 100, or 90-100 |
| ~N410 + Deamidation | 1-25, |
| N479 + Deamidation | 1-5, 1-3 |
| ~N514 + Deamidation | 65-90, 70-95, 80- |
| | 95, 75-100, 80- |
| | 100, or 90-100 |
| ~Q601 + Deamidation | 0-1 |
| N653 + Deamidation | 0-2 |

In certain embodiments, an AAVrh79 capsid is modified in one or more of the positions identified in the preceding table, in the ranges provided below, as determined using mass spectrometry with a trypsin enzyme. In certain embodiments, one or more of the following positions, or the glycine following the N is modified as described herein. Residue numbers are based on the AAVrh79 sequence provided herein. See, SEQ ID NO: 34.

In certain embodiments, the nucleic acid sequence encoding the AAVrh79 vp1 capsid protein is provided in SEQ ID NO: 33. In other embodiments, a nucleic acid sequence of 70% to 99.9% identity to SEQ ID NO: 33 may be selected to express the AAVrh79 capsid proteins. In certain other embodiments, the nucleic acid sequence is at least about 75% identical, at least 80% identical, at least 85%, at least 90%, at least 95%, at least 97% identical, at least 99% or at least 99.9% identical to SEQ ID NO: 33. However, other nucleic acid sequences which encode the amino acid sequence of SEQ ID NO: 34 may be selected for use in producing rAAV capsids. In certain embodiments, the nucleic acid sequence has the nucleic acid sequence of SEQ ID NO: 33 or a sequence at least 70% to 99% identical, at least 75%, at least 80%, at least 85%, at least 90%, at least 95%, at least 97%, at least 99%, identical to SEQ ID NO: 33 which encodes SEQ ID NO: 34. In certain embodiments, the nucleic acid sequence has the nucleic acid sequence of SEQ ID NO: 33 or a sequence at least 70% to 99. %, at least 75%, at least 80%, at least 85%, at least 90%, at least 95%, at least 97%, at least 99%, identical to about nt 412 to about nt 2214 of SEQ ID NO: 33 which encodes the vp2 capsid protein (about aa 138 to 738) of SEQ ID NO: 34. In certain embodiments, the nucleic acid sequence has the nucleic acid sequence of about nt 610 to about nt 2214 of SEQ ID NO: 33 or a sequence at least 70% to 99. %, at least 75%, at least 80%, at least 85%, at least 90%, at least 95%, at least 97%, at least 99%, identical to nt SEQ ID NO: 33 which encodes the vp3 capsid protein (about aa 204 to 738) of SEQ ID NO: 34.

The invention also encompasses nucleic acid sequences encoding mutant AAVrh79, in which one or more residues has been altered in order to decrease deamidation, or other modifications which are identified herein. Such nucleic acid sequences can be used in production of mutant rAAVrh79 capsids.

In certain embodiments, the rAAV comprises a AAVrh.90 capsid, as described in WO 2020/223232, published Nov. 5, 2020, which is incorporated herein by reference In a further aspect, a recombinant adeno-associated virus (rAAV) is provided which comprises: (A) an AAVrh.90 capsid comprising one or more of: (1) AAVrh.90 capsid proteins comprising: a heterogeneous population of AAVrh.90 vp1 proteins selected from: vp1 proteins produced by expression from a nucleic acid sequence which encodes the predicted amino acid sequence of 1 to 738 of SEQ ID NO: 40, vp1 proteins produced from SEQ ID NO: 39, or vp1 proteins produced from a nucleic acid sequence at least 70% identical to SEQ ID NO: 39 which encodes the predicted amino acid sequence of 1 to 738 of SEQ ID NO: 40, a heterogeneous population of AAVrh.90 vp2 proteins selected from: vp2 proteins produced by expression from a nucleic acid sequence which encodes the predicted amino acid sequence of at least about amino acids 138 to 738 of SEQ ID NO: 40, vp2 proteins produced from a sequence comprising at least nucleotides 412 to 2214 of SEQ ID NO: 39, or vp2 proteins produced from a nucleic acid sequence at least 70% identical to at least nucleotides 412 to 2214 of SEQ ID NO: 39 which encodes the predicted amino acid sequence of at least about amino acids 138 to 738 of SEQ ID NO: 40, a heterogeneous population of AAVrh.90 vp3 proteins selected from: vp3 proteins produced by expression from a nucleic acid sequence which encodes the predicted amino acid sequence of at least about amino acids 204 to 738 of SEQ ID NO: 40, vp3 proteins produced from a sequence comprising at least nucleotides 610 to 2214 of SEQ ID NO: 39, or vp3 proteins produced from a nucleic acid sequence at least 70% identical to at least nucleotides 610 to 2214 of SEQ ID NO: 39 which encodes the predicted amino acid sequence of at least about amino acids 204 to 738 of SEQ ID NO: 40; and/or (2) a heterogeneous population of vp1 proteins which are the product of a nucleic acid sequence encoding the amino acid sequence of SEQ ID NO: 40, a heterogeneous population of vp2 proteins which are the product of a nucleic acid sequence encoding the amino acid sequence of at least about amino acids 138 to 738 of SEQ ID NO: 40, and a heterogeneous population of vp3 proteins which are the product of a nucleic acid sequence encoding at least amino acids 204 to 738 of SEQ ID NO: 40, wherein: the vp1, vp2 and vp3 proteins contain subpopulations with amino acid modifications comprising at least two highly deamidated asparagines (N) in asparagine-glycine pairs in SEQ ID NO: 40 and optionally further comprising subpopulations comprising other deamidated amino acids, wherein the deamidation results in an amino acid change; and (B) a vector genome in the AAVrh. 90 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the AAVrh.90 vp1, vp2 and vp3 proteins contain subpopulations with amino acid modifications comprising at least two highly deamidated asparagines (N) in asparagine-glycine pairs in SEQ ID NO: 40 and optionally further comprising subpopulations comprising other deamidated amino acids, wherein the deamidation results in an amino acid change. High levels of deamidation at N-G pairs N57, ˜N263, ˜N385, and/or ˜N514 are observed, relative to the number of SEQ ID NO: 40. Deamidation has been observed in other residues as shown in the table below. In certain embodiments, AAVrh.90 may have other residues deamidated (e.g., ˜N305, ˜N499, and/or ˜N599, typically at less than 20%) and/or may have other modifications, including phosphorylation (e.g., where present, in the range of about 2 to about 30%, or about 2 to about 20%, or about 2 to about 10%) (e.g., at S149), or oxidation (e.g. at one or more of ˜W23, ˜M204, ˜M212, W248, W282, M405, M473, W480, W505, M526, ˜N544, M561, and/or ˜M607). Optionally the W may oxidize to kynurenine.

TABLE D

| AAVrh.90 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| ~N263 + Deamidation | 65-90, 70-95, 80-95, 75-100, 80-100, or 90-100 |
| ~N305 + Deamidation | 5-30, 5-20, or 10-20 |
| ~N385 + Deamidation | 65-90, 70-95, 80-95, 75-100, 80-100, or 90-100 |
| ~N499 + Deamidation | 2-15, 2-10, or 5-10 |
| ~N514 + Deamidation | 65-90, 70-95, 80-95, 75-100, 80-100, or 90-100 |
| ~N599 + Deamidation | 2-15, 2-10, or 5-10 |

In certain embodiments, an AAVrh.90 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh.90 sequence provided herein. See, SEQ ID NO: 40.

In certain embodiments, an AAVrh.90 capsid comprises: a heterogeneous population of vp1 proteins which are the product of a nucleic acid sequence encoding the amino acid sequence of SEQ ID NO: 40, a heterogeneous population of vp2 proteins which are the product of a nucleic acid sequence encoding the amino acid sequence of at least about amino acids 138 to 738 of SEQ ID NO: 40, and a heterogeneous population of vp3 proteins which are the product of a nucleic acid sequence encoding at least amino acids 204 to 738 of SEQ ID NO: 40.

In certain embodiments, the parvovirus vector capsids are selected for liver-tropism. As used herein, a “vector genome” refers to the nucleic acid sequence packaged inside the rAAV capsid which forms a viral particle. Such a nucleic acid sequence contains AAV inverted terminal repeat sequences (ITRs). In certain embodiments, the vector genome in the packaged capsid may be “partial”, e.g., lacking one or both ITRs, but still functional. In certain embodiments, a vector genome contains from 5′ to 3″, an AAV 5′ ITR, expression cassette containing the transgene or coding sequence(s) operably linked to regulatory sequences directing expression thereof, and an AAV 3′ ITR. The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (ΔITR), which may be used for convenience. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external “a” element is deleted. The shortened ITR is reverted back to the wild-type length of 145 base pairs during vector DNA amplification using the internal A element as a template. In other embodiments, the full-length AAV 5′ and 3′ ITRs are used. In other embodiments, a full-length or engineered ITR may be selected. ITRs from AAV2, a different source AAV than the capsid, or other than full-length ITRs may be selected. The ITRs are from the same AAV source as the AAV which provides the rep function during production or a transcomplementing AAV. Further, other ITRs may be used. Examples of suitable ITR sequences are shown in the sequence listing, e.g., SEQ ID NO: 42, nt 1 to 130 and 3052 to 3181. Further, the vector genome contains regulatory sequences that direct modulate expression of the gene products (e.g., directly or indirectly by modulating transcription and/or translation). Suitable components of a vector genome are discussed in more detail herein.

In certain embodiments, the gene editing vector genome includes a TBG promoter, one or more alpha mic/bik enhancer(s), coding sequence for the ARCUS meganuclease, optionally a WPRE, and a polyA. In certain embodiments, the expression cassette includes nt 211 to nt 2964 of SEQ ID NO: 42, flanked by 5′ and 3′ ITRs.

In certain embodiments, the gene editing component includes a U6 promoter, one or more alpha mic/bik enhancer(s), Cas9 coding sequence, sgRNA targeting PCSK9, hybrid liver promoter, and a polyA.

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. Exemplary production plasmids are shown in SEQ ID Nos: 1 and 4.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin Biotechnol. 99: 119-145: Buning et al. 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397: WO 2005/033321, WO 2006/110689: U.S. Pat. No. 7,588,772 B2. Such a method involves culturing a host cell which contains a nucleic acid sequence encoding an AAV capsid protein: a functional rep gene: an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell: a nucleic acid molecule suitable for packaging into the AAV capsid, e.g., a vector genome which contains AAV ITRs and a non-AAV nucleic acid sequence encoding a gene product operably linked to sequences which direct expression of the product in a host cell; and sufficient AAV rep functions and adenovirus helper functions to permit packaging of the nucleic acid molecule into the recombinant AAV capsid. In one embodiment, the cell culture is composed of mammalian cells (e.g., human embryonic kidney 293 cells, among others) or insect cells (e.g., baculovirus).

Optionally the rep functions are provided by an AAV other than the AAV providing the capsid. For example, the rep may be, but is not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof: or another source. Optionally, the rep and cap sequences are on the same genetic element in the cell culture. There may be a spacer between the rep sequence and cap gene. Any of these AAV or mutant AAV capsid sequences may be under the control of exogenous regulatory control sequences which direct expression thereof in a host cell.

In one embodiment, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: 5,139,941:5,741, 683; 6,057, 152; 6,204,059; 6,268,213:6,491,907:6,660,514; 6,951,753:7,094,604; 7,172,893:7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Publication No. WO 2017/160360, which is incorporated by reference herein. Purification methods for AAV8, International Patent Publication No. WO 2017/100676, and rh 10. International Patent Publication No. WO 2017/100704, and for AAV1. International

Patent Publication No. WO 2017/100674 are all incorporated by reference herein.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the BI anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using Silver Xpress (Invitrogen. CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al. Hu Gene Therapy Methods, Hum Gene Ther Methods. 2014 April: 25 (2): 115-25, doi: 10.1089/hgtb.2013.131. Epub 2014 Feb. 14. The ddPCR method directly measures the concentration of encapsidated vector genomes. The sample is treated with DNase I to digest any non-encapsidated DNA present in the sample followed by treatment with Proteinase K to disrupt the capsid. The sample is then diluted to fit the assay range. The sample is mixed with ddPCR Supermix, and detection is accomplished using sequence-specific primers targeting the Meganuclease specific to the PCSK9 gene (M2PCSK9) in combination with a fluorescently-tagged probe hybridizing to this same region. Twenty microliters of ddPCR reaction mixture are processed in the Bio-Rad droplet generator, and the ddPCR reaction mixture is partitioned into ≥10,000 droplets. After droplet generation, the ddPCR reaction mixture undergoes PCR amplification, and the amplified ddPCR reaction mixture is read using a Bio-Rad Droplet Reader.

The infectious unit (IU) assay may be used to determine the productive uptake and replication of rAAV vector in RC32 cells (rep2 expressing HeLa cells). A 96-well endpoint format has been employed similar to that previously published. Briefly, RC32 cells will be co-infected by serial dilutions of rAAV BDS and a uniform dilution of Ad5 with 12 replicates at each dilution of rAAV. Seventy-two hours after infection, the cells will be lysed, and qPCR will be performed to detect rAAV vector amplification over input. An endpoint dilution 50% tissue culture infectious dose (TCID50) calculation (Spearman-Karber) will be performed to determine a infectious titer expressed as IU/mL. Since “infectivity” values are dependent on each particle's contact with cells, receptor binding, internalization, transport to the nucleus, and genome replication, they are influenced by assay geometry and the presence of appropriate receptors and post-binding pathways in the cell line used. Receptors and post-binding pathways are not usually maintained in immortalized cell lines, and thus infectivity assay titers are not an absolute measure of the number of “infectious” particles present. However, the ratio of encapsidated GC to “infectious units” (described as GC/IU ratio) can be used as a measure of product consistency from lot to lot.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high pH, and subjected to a salt gradient while monitoring eluate for ultraviolet absorbance at about 260 and about 280. The pH may be adjusted depending upon the AAV selected. See, e.g., WO2017/160360 (AAV9), WO2017/100704 (AA Vrh10). WO 2017/100676 (e.g., AAV8), and WO 2017/100674 (AAV1)] which are incorporated by reference herein. In this method, the AAV full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2 serotype. Under these ionic conditions, a significant percentage of residual cellular DNA and proteins flow through the column, while AAV particles are efficiently captured.

### Dual Component System

In another aspect, a dual component system for treating a genetic disorder is provided. The system includes (a) a gene editing component that includes a nucleic acid sequence encoding a nuclease that targets PCSK9 and, optionally, regulatory sequences that direct expression of the nuclease in a target cell comprising a PCSK9 gene; and (b) a donor vector comprising a nucleic acid sequence encoding an exogenous product for expression from the PCSK9 locus, wherein the inserted nucleic acid sequence does not encode PCSK9, and wherein the system further comprises sequences that direct the nuclease to specifically targets the native PCSK9 gene locus. The system optionally comprises a component which allows the native PCSK9 in the target cell to be ablated or reduced post-dosing with the dual component system, e.g., via use of an inducing agent with an inducible promoter. In one embodiment the gene editing component is comprised in a gene editing vector comprising an expression cassette comprising a nucleic acid sequence encoding a nuclease and regulatory sequences that direct expression of the nuclease in a target cell comprising a PCSK9 gene. The components of the dual component system are as those described herein. It is noted that each “component” of the dual component system may comprise or consist of one or more elements.

While the system can be effective if the ratio of gene editing component to donor vector is about 1 to about 1, it is desirable for the donor template vector to be present in excess of the gene editing component. In one embodiment, the ratio of editing vector (a) to donor vector (b) is about 1:3 to about 1:100, or about 1:10. In certain embodiments, the ratio of editing vector (a) to donor vector (b) is about 1:3. In certain embodiments, the ratio of editing vector (a) to donor vector (b) is about 1:2. In certain embodiments, the ratio of editing vector (a) to donor vector (b) is about 1:2.5. In certain embodiments, the ratio of editing vector (a) to donor vector (b) is about 1:3.5. In certain embodiments, the ratio of editing vector (a) to donor vector (b) is about 1:4. In certain embodiments, the ratio of editing vector (a) to donor vector (b) is about 1:4.5. In certain embodiments, the ratio of editing vector (a) to donor vector (b) is about 1:5. This ratio of gene editing enzyme (e.g., Cas9 or meganuclease) to donor template may be maintained even if the enzyme is additionally or alternatively supplied by a source other than the AAV vector.

In one embodiment, the dual component system includes a gene editing AAV vector comprising an AAV capsid and a first vector genome comprising a 5′ ITR, a sequence encoding a meganuclease that targets PCSK9 under control of regulatory sequences that direct expression of the meganuclease in a target cell comprising a PCSK9 gene, and a 3′ ITR; and (b) a donor AAV vector comprising an AAV capsid and a second vector genome comprising: a 5′ITR, a 5′ homology directed recombination (HDR) arm, a Factor IX transgene and regulatory sequences that direct expression of the transgene in the target cell, a 3′ HDR arm, and a 3′ ITR.

In another embodiment, the dual component system includes a gene editing AAV comprising an AAV capsid and a first vector genome comprising a 5′ ITR, a 5′ nuclear localization signal (NLS), a sequence encoding a Cas9 and regulatory sequences that direct expression of the SaCas9 in a target cell comprising the PCSK9 gene, a 3′ NLS, and a 3′ ITR; and a donor AAV vector comprising an AAV capsid and a second vector genome comprising: a 5′ITR, a 5′ homology directed recombination (HDR) arm, a Factor IX transgene and regulatory sequences that direct expression of the transgene in the target cell, a 3′ HDR arm, a U6 promoter, a sgRNA comprising at least 20 nucleotides that specifically bind to a target site in the PCSK9 gene, said target site being 5′ to a protospacer-adjacent motif (PAM) that is specifically recognized by the Cas9, and a 3′ ITR. In one embodiment, the gene editing vector has a vector genome comprising SEQ ID NO: 5.

In another embodiment, the dual component system includes a gene editing AAV vector comprising an AAV capsid and a first vector genome comprising a 5′ ITR, a U6 promoter, a sgRNA comprising at least 20 nucleotides that specifically bind to a target site in the PCSK9 gene, said target site being 5′ to a protospacer-adjacent motif (PAM) that is specifically recognized by the Cas9, a 5′ nuclear localization signal (NLS), a sequence encoding a Cas9 and regulatory sequences that direct expression of the Cas9 in a target cell comprising the PCSK9 gene, a 3′ NLS, and a 3′ ITR; and a donor AAV vector comprising an AAV capsid and a second vector genome comprising: a 5′ITR, a 5′ homology directed recombination (HDR) arm, a Factor IX transgene and regulatory sequences that direct expression of the transgene in the target cell, a 3′ HDR arm, and a 3′ ITR.

In certain embodiments of the systems described herein, the gene editing AAV vector the donor AAV vector have the same AAV capsid. In other embodiments, the gene editing AAV vector and the donor AAV vector have different AAV capsids. In some embodiments, the AAV capsid is selected from AAV8, AAV9, rh10, AAV6.2, AAV3B, hu37, rh79, and rh64. In some embodiments, the donor and/or gene editing vector has a hu37 capsid. In some embodiments, the donor and/or gene editing vector has an rh79 capsid.

In certain embodiments, the nuclease is a Cas9 nuclease, and the Cas9 is selected from Staphylococcus aureus or Streptococcus pyogenes Cas9. An amino acid sequence for SaCas9 is shown in SEQ ID NO: 6. A coding sequence for SaCas9 is shown in SEQ ID NO: 7.

In one embodiment, the gene editing vector has a vector genome comprising SEQ ID NO: 2. In one embodiment, the gene editing vector has a vector genome comprising SEQ ID NO: 5.

In certain embodiments, the nuclease and/or transgene is under the control of a tissue-specific promoter. In certain embodiments, the nuclease and/or transgene is under the control of a constitutive promoter. In certain embodiments, the nuclease and/or transgene is under the control of an inducible promoter. In certain embodiments, the nuclease and/or transgene is under the control of a liver-specific promoter, optionally a human thyroxin-binding globulin (TBG) promoter, or hybrid liver promoter (HLP). In certain embodiments, the system further comprises an inducing agent.

In another embodiment, the system includes (a) a gene editing component that includes a nucleic acid sequence encoding a nuclease that targets PCSK9 and regulatory sequences that direct expression of the nuclease in a target cell comprising a PCSK9 gene encapsulated in a LNP; and (b) a donor vector comprising a nucleic acid sequence encoding Factor IX for expression from the PCSK9 locus encapsulated in a LNP, wherein the system further comprises sequences that direct the nuclease to specifically target the native PCSK9 gene locus. The system optionally comprises a component which allows the native PCSK9 in the target cell to be ablated or reduced post-dosing with the dual component system, e.g., via use of an inducing agent with an inducible promoter.

In another embodiment, the system includes (a) a gene editing component that includes a nucleic acid sequence encoding a nuclease that targets PCSK9 and regulatory sequences that direct expression of the nuclease in a target cell comprising a PCSK9 gene wherein the gene editing component is provided via AAV vector; and (b) a donor vector comprising a nucleic acid sequence encoding Factor IX for expression from the PCSK9 locus encapsulated in a LNP, wherein the system further comprises sequences that direct the nuclease to specifically targets the native PCSK9 gene locus. The system optionally comprises a component which allows the native PCSK9 in the target cell to be ablated or reduced post-dosing with the dual component system, e.g., via use of an inducing agent with an inducible promoter.

In another embodiment, the system includes (a) a gene editing component that includes a nucleic acid sequence encoding a nuclease that targets PCSK9 and regulatory sequences that direct expression of the nuclease in a target cell comprising a PCSK9 gene encapsulated in a LNP; and (b) a donor vector comprising a nucleic acid sequence encoding Factor IX for expression from the PCSK9 locus, wherein the system further comprises sequences that direct the nuclease to specifically targets the native PCSK9 gene locus. The system optionally comprises a component which allows the native PCSK9 in the target cell to be ablated or reduced post-dosing with the dual component system, e.g., via use of an inducing agent with an inducible promoter.

In one embodiment, the dual component system includes (a) a LNP comprising mRNA that encodes a meganuclease that targets PCSK9 under control of regulatory sequences that direct expression of the meganuclease in a target cell comprising a PCSK9 gene; and (b) a donor AAV vector comprising an AAV capsid and a second vector genome comprising: a 5′ITR, a 5′ homology directed recombination (HDR) arm, a Factor IX transgene and regulatory sequences that direct expression of the transgene in the target cell, a 3′ HDR arm, and a 3′ ITR.

In another embodiment, the dual component system includes (a) a LNP comprising a nucleic acid comprising a sequence encoding a Cas9 and a sgRNA comprising at least 20 nucleotides that specifically bind to a target site in the PCSK9 gene, said target site being 5′ to a protospacer-adjacent motif (PAM) that is specifically recognized by the Cas9; and (b) a donor AAV vector comprising an AAV capsid and a vector genome comprising: a 5′ITR, a 5′ homology directed recombination (HDR) arm, a Factor IX transgene and regulatory sequences that direct expression of the transgene in the target cell, a 3′ HDR arm, and a 3′ ITR. The sequence encoding Cas9 is provided as mRNA.

In another embodiment, the dual component system includes a gene editing AAV vector comprising an AAV capsid and a first vector genome comprising a 5′ ITR, a U6 promoter, a sgRNA comprising at least 20 nucleotides that specifically bind to a target site in the PCSK9 gene, said target site being 5′ to a protospacer-adjacent motif (PAM) that is specifically recognized by the Cas9, a 5′ nuclear localization signal (NLS), a sequence encoding a Cas9 and regulatory sequences that direct expression of the Cas9 in a target cell comprising the PCSK9 gene, a 3′ NLS, and a 3′ ITR; and a donor AAV vector comprising an AAV capsid and a second vector genome comprising: a 5′ITR, a 5′ homology directed recombination (HDR) arm, a Factor IX transgene and regulatory sequences that direct expression of the transgene in the target cell, a 3′ HDR arm, and a 3′ ITR.

### Pharmaceutical Compositions

In another aspect, a pharmaceutical composition is provided which contains a first rAAV stock comprising rAAV gene editing vectors comprising an expression cassette comprising a nucleic acid sequence encoding a nuclease that targets PCSK9 and regulatory sequences that direct expression of the nuclease in a target cell comprising a PCSK9 gene: and a second rAAV stock comprising rAAV donor vectors comprising a Factor IX transgene cassette comprising a nucleic acid sequence encoding a transgene and regulatory sequences that direct expression of the transgene in the target cell. The pharmaceutical composition contains an optional carrier, excipient, and/or preservative. In some embodiments, the donor vector further includes homology-directed recombination (HDR) arms 5′ and 3′ to the transgene cassette. In one embodiment, the AAV capsid for the donor vector, gene editing vector, or both, is an AAVrh79 capsid. In another embodiment, the AAV capsid for the donor vector, gene editing vector, or both, is an AAVrh.90 capsid. In another embodiment, the AAV capsid for the donor vector, gene editing vector, or both, is an AAVhu.37 capsid. In one embodiment, the AAV capsid for the donor vector, gene editing vector, or both, is an AAV8 capsid. In one embodiment, the AAV capsid for the donor vector, gene editing vector, or both, is an AAVrh.91 capsid. In one embodiment, the AAV capsid for the donor vector, gene editing vector, or both, is an AAVhu.68 capsid.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

Methods and agents well known in the art for making formulations are described, for example, in “Remington's Pharmaceutical Sciences,” Mack Publishing Company, Easton, Pa. Formulations may, for example, contain excipients, carriers, stabilizers, or diluents such as sterile water, saline, polyalkylene glycols such as polyethylene glycol, oils of vegetable origin, or hydrogenated napthalenes, preservatives (such as octadecyldimethylbenzyl, ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol), low molecular weight polypeptides, proteins such as serum albumin, gelatin, or immunoglobulins, hydrophilic polymers such as polyvinylpyrrolidone, amino acids such as glycine, glutamine, asparagine, histidine, arginine, and lysine, monosaccharides, disaccharides, and other carbohydrates including glucose, mannose, and dextrins, chelating agents such as EDTA, sugars such as sucrose, mannitol, trehalose or sorbitol: salt-forming counter-ions such as sodium; metal complexes (e.g. Zn-protein complexes); and/or non-ionic surfactants such as TWEEN™, PLURONICS™ or polyethylene glycol (PEG).

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery), lung, heart, eye, kidney.), oral, inhalation, intranasal, intrathecal, intratracheal, intraarterial, intraocular, intravenous, intramuscular, subcutaneous, intradermal, and other parental routes of administration. Routes of administration may be combined, if desired.

Dosages of the viral vector depend primarily on factors such as the condition being treated, the age, weight and health of the patient, and may thus vary among patients. For example, a therapeutically effective human dosage of the viral vector is generally in the range of from about 25 to about 1000 microliters to about 100 mL of solution containing concentrations of from about 1×10⁹ to 1×10¹⁶ genomes virus vector. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The vector compositions can be formulated in dosage units to contain an amount of replication-defective virus that is in the range of about 1.0×10⁹ GC to about 1.0×1016 GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. Any suitable route of administration may be selected. Accordingly, pharmaceutical compositions may be formulated for any appropriate route of administration, for example, in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

### Methods

The compositions provided herein are useful for treatment hemophilia B. Provided herein is a method of treating a disorder in a human by co-administering the dual component system as described herein.

In one embodiment, a method of treating hemophilia B in an adolescent subject is provided. The method includes co-administering to the subject having hemophilia B a gene editing AAV vector comprising a sequence encoding a nuclease that targets PCSK9 and regulatory sequences that direct expression of the nuclease in a target cell comprising a PCSK9 gene; and a donor AAV vector comprising a factor IX transgene and regulatory sequences that direct expression of the transgene in the target cell. In another embodiment, the method includes co-administering to the subject having a hemophilia B an LNP comprising a sequence encoding a Cas9 nuclease and sgRNA that target PCSK9 in a target cell comprising a PCSK9 gene; and a donor AAV vector comprising a factor IX transgene and regulatory sequences that direct expression of the transgene in the target cell.

In one embodiment, a method of treating hemophilia B in an adult subject is provided. The method includes co-administering to the subject having hemophilia B a gene editing AAV vector comprising a sequence encoding a nuclease that targets PCSK9 and regulatory sequences that direct expression of the nuclease in a target cell comprising a PCSK9 gene; and a donor AAV vector comprising a factor IX transgene and regulatory sequences that direct expression of the transgene in the target cell. In another embodiment, the method includes co-administering to the subject having a hemophilia B an LNP comprising a sequence encoding a Cas9 nuclease and sgRNA that target PCSK9 in a target cell comprising a PCSK9 gene; and a donor AAV vector comprising a factor IX transgene and regulatory sequences that direct expression of the transgene in the target cell.

In one embodiment, a method of treating hemophilia B in a subject is provided. The method includes co-administering to the subject having hemophilia B a gene editing AAV vector comprising a sequence encoding a nuclease that targets PCSK9 and regulatory sequences that direct expression of the nuclease in a target cell comprising a PCSK9 gene; and a donor AAV vector comprising a factor IX transgene and regulatory sequences that direct expression of the transgene in the target cell. In another embodiment, the method includes co-administering to the subject having a hemophilia B an LNP comprising a sequence encoding a Cas9 nuclease and sgRNA that target PCSK9 in a target cell comprising a PCSK9 gene; and a donor AAV vector comprising a factor IX transgene and regulatory sequences that direct expression of the transgene in the target cell.

In certain of the methods described herein, the native PCSK9 expression is reduced or ablated and a transgene is expressed from the insertion in the native PCSK9 locus. In another embodiment, the native PCSK9 expression is reduced or ablated, and the transgene is expressed exogenously, i.e., without being integrated into the subject's genome.

In certain embodiment, the subject is older than an infant. In certain embodiments, the subject is a child. In other embodiments, the subject is ≥6 to <12 years of age. In certain embodiments, the subject is an adolescent. In certain embodiments, the adolescent is ≥12 to <18 years of age. In another embodiment, the subject is an adult. In certain embodiments, the subject is greater than 18 years of age. In certain embodiments, the subject is a young adult, e.g., under 30 years of age. In certain embodiments, the subject is age 20 or greater. In certain embodiments, the subject is age 25 or greater. In certain embodiments, the subject is age 30 or greater. In certain embodiments, the subject is age 35 or greater. In certain embodiments, the subject is age 40 or greater. In certain embodiments, the subject is age 45 or greater. In certain embodiments, the subject is age 50 or greater. In certain embodiments, the subject is age 55 or greater. In certain embodiments, the subject is age 60 or greater. In certain embodiments, the subject is age 65 or greater.

In certain embodiments, the gene editing AAV vector and the donor vector of are delivered essentially simultaneously via the same route. In other embodiments, the gene editing vector is delivered first. In other embodiments, the donor vector is delivered first.

In one embodiment, the dosage of an rAAV is about 1×10⁹ GC to about 1×10¹⁵ genome copies (GC) per dose (to treat an average subject of 70 kg in body weight), and preferably 1.0×10¹² GC to 2.0×10¹⁵ GC for a human patient. In another embodiment, the dose is less than about 1×10¹⁴ GC/kg body weight of the subject. In certain embodiments, the dose administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10¹⁰ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×1010 GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×1011 GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×1013 GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg body weight or the subject.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Desirable routes of administration include direct delivery to a desired organ (e.g., the liver (optionally via the hepatic artery), lung, heart, eye, kidney), oral, inhalation, intranasal, intratracheal, intrathecal, intraarterial, intraocular, intravenous, intramuscular, subcutaneous, intradermal, and other parental routes of administration. Routes of administration may be combined, if desired.

The system described herein may be therapeutically useful if a sufficient amount of functional protein is generated to improve the patient's condition. In certain embodiments, gene expression levels as low as 5% of healthy patients will provide sufficient therapeutic effect for the patient to be treatable by non-gene therapy approaches. In other embodiments, gene expression levels are at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to up to 100% of the normal range (levels) observed in humans (or other veterinary subject). For example, by “functional protein”, is meant a gene which encodes the wild-type enzyme or functional variant (e.g., Factor IX) which provides at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about the same, or greater than 100% of the biological activity level of the wild-type protein, or a natural variant or polymorph thereof which is not associated with disease. More particularly, as heterozygous patients may have as low a functional level as about 50% or lower, effective treatment may not require replacement to levels within the range of “normal” or non-deficient patients. Similarly, patients having no detectable amounts of Factor IX may be rescued by delivering levels to less than 100%, and may optionally be subject to further treatment subsequently. In certain embodiments, where gene function is being delivered by the donor template, patients may express higher levels than found in “normal”, healthy subjects. As described herein, the therapy described herein may be used in conjunction with other treatments, i.e., the standard of care for the subject's (patient's) diagnosis.

In one embodiment, the method further comprises administering an immunosuppressive co-therapy to the subject. Such immunosuppressive co-therapy may be started prior to delivery of an rAAV or a composition as disclosed, e.g., if undesirably high neutralizing antibody levels to the AAV capsid are detected. In certain embodiments, co-therapy may also be started prior to delivery of the rAAV as a precautionary measure. In certain embodiments, immunosuppressive co-therapy is started following delivery of the rAAV, e.g., if an undesirable immune response is observed following treatment.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include prednisolone, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the rAAV administration, or 0, 1, 2, 3, 7, or more days post the rAAV administration. Such therapy may involve a single drug (e.g., prednisolone) or co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), two weeks, three weeks, about 60 days, or longer, as needed. In certain embodiments, a tacrolimus-free regimen is selected.

In another embodiment, the method includes co-treatment with a standard hemophilia therapy. The severity of hemophilia B is defined by the level of circulating FIX and management is based on FIX replacement therapy administered either prophylactically to prevent bleeding episodes or after a bleeding episode has occurred, known as “on-demand” treatment. The standard of care for patients with severe and moderately severe hemophilia B is FIX prophylaxis. FIX supplementation is given intravenously as using standard half-life (SHL) or extended half-life (EHL) treatments, which are given every 2-3 or 7-14 days, respectively. The frequent infusions required by FIX treatment incur a level of treatment burden that can compromise adherence and clinical effectiveness. People with mild or moderate hemophilia B who tend to experience relatively infrequent bleeding episodes are often managed with on-demand FIX treatment to minimize treatment burden. n the U.S., the currently available recombinant factor IX products are BeneFIX, Rixubis, Ixinity. Alprolix Idelvion, and Rebinyn. Other treatments available on the market include prothrombin complex concentrates (PCCs), which contain prothrombin, factors VII and X, and proteins C and S, in addition to factor IX. PCCs may also contain small amounts of factors VIIa, IXa, and Xa.

In another embodiment, the method further comprises administering at least one anti-FcRn ligand (e.g., anti-FcRn antibody) to permit effective vector delivery. In certain embodiments, the methods comprising administering anti-FcRn ligands and the regimens and co-administration are utilized during systemic delivery of viral vectors.

As used herein, the term “FcRn” refers a neonatal Fc receptor that binds to the Fc region of an immunoglobulin (IgG) antibody. An exemplary FcRn is human FcRn having UniProt ID No. P55899. Human FcRn is believed to be responsible for maintaining the half-life of IgG by binding and trafficking constitutively internalized IgG back to the cell surface for the recycling of IgG. Unless otherwise specified, “FcRn” refers to a patient's native FcRn.

As used herein, an “FcRn ligand” is any moiety (e.g., without limitation, peptide, protein, antibody, a shRNA, RNAi, a nucleic acid encoding a peptide, protein, or antibody, or small molecule drug) which blocks or significantly reduces binding between human neonatal Fc receptor (FcRn) and a patient's neutralizing antibodies. In desired embodiments, the ligand may be referred to herein as “anti-FcRn”. In certain embodiments, the FcRn ligand blocks FcRn binding to a patient's NAbs without blocking FcRn binding to albumin. This may be referred to herein as an FcRn-IgG blocking ligand, an FcRn-NAb blocking ligand, or an anti-FcRn ligand.

In certain embodiments, the anti-FcRN ligand is a monoclonal antibody. Examples of such antibodies may include, e.g., nipocalimab (M281) (Momenta Pharmaceuticals Inc), rozanolixizumab (UCB7665) (UCB SA); IMVT-1401, RVT-1401 (HL161), HBM9161 (all form HanAll BioPhrma Co. Ltd), ARGX-113 (efgartigimod) (Argenx S.E.), orilanolimab (ALXN 1830, SYNT001, Alexion Pharmaceuticals Inc), SYNT002, ABY-039 (Affibody AB), or DX-2507 (Takeda Pharmaceutical Co. Ltd), combinations thereof, or one of these antibodies in combination with another ligand. Alternatively, other antibody constructs may be derived from these antibodies, among others.

As used herein, the term “inhibit IgG binding to FcRn” refers to the ability of a ligand to block or inhibit the binding of IgG (e.g., IgG1) to a patient's native FcRn (e.g., human FcRn in a human patient). In some embodiments, the ligand binds FcRn, for example, at the site on human FcRn to which IgG binds. Thus, the ligand inhibits the binding of a patient's IgG autoantibodies to FcRn. In some embodiments, the ligand substantially or completely inhibits binding to IgG. In some embodiments, the binding of IgG is reduced by 10%, 20%, 30%, 50%, 70%, 80%, 90%, 95%, or even 100%.

Other such methods and compositions comprising co-administration of an FcRn ligand with a vector system are known by the skilled artisan including, for example in WO 2021/257668 A1 which is incorporated herein by reference.

In another embodiment, the method further comprises administering at least one anti-CD20 ligand (e.g., anti-CD20 antibody) to permit effective vector delivery. In certain embodiments, the methods comprising administering anti-CD20 ligands and the regimens and co-administration are utilized during systemic delivery of viral vectors. Such ligands may include or be derived from antibodies such as rituximab (rituzin, a chimeric anti-CD20), daclizumab (Zenepax, a humanized anti-CD25), ocrelizumab (humanized, anti-CD20, Roche), “C2B8” (U.S. Pat. No. 5,736,137); or tositumomab (BEXXAR). In one embodiment, the CD20 ligand is rituximab.

In one aspect, a method is provided for treating a patient having hemophilia B, using a nuclease expression cassette comprising a meganuclease coding sequence that recognizes a site within the human PCSK9 gene, under the control of a promoter as described herein.

In another aspect, a method is provided for treating a patient having hemophilia B, using a nuclease expression cassette comprising a an sgRNA and Cas9 coding sequence that recognizes a site within the human PCSK9 gene. The method further includes administration of an expression cassette carrying a Factor IX transgene, as described herein. Such expression cassettes may be delivered via a viral or non-viral vector. In certain embodiments, the expression cassettes may be delivered using an LNP.

A variety of assays exist for measuring OTC expression and activity levels in vitro. See, e.g., X Ye, et al, 1996 Prolonged metabolic correction in adult ornithine transcarbamylase-deficient mice with adenoviral vectors. J Biol Chem 271:3639-3646) or in vivo. For example, OTC enzyme activity can be measured using a liquid chromatography mass spectrometry stable isotope dilution method to detect the formation of citrulline normalized to [1,2,3,4,5-13C5] citrulline (98% 13C). The method is adapted from a previously developed assay for detection of N-acetylglutamate synthase activity [Morizono H, et al, Mammalian N-acetylglutamate synthase. Mol Genet Metab. 2004; 81(Suppl 1):S4-11.]. Slivers of fresh frozen liver are weighed and briefly homogenized in buffer containing 10 mM HEPES, 0.5% Triton X-100, 2.0 mM EDTA and 0.5 mM DTT. Volume of homogenization buffer is adjusted to obtain 50 mg/ml tissue. Enzyme activity is measured using 250 ug liver tissue in 50 mM Tris-acetate, 4 mM ornithine, 5 mM carbamyl phosphate, pH 8.3. Enzyme activity is initiated with the addition of freshly prepared 50 mM carbamyl phosphate dissolved in 50 mM Tris-acetate pH 8.3, allowed to proceed for 5 minutes at 25° C. and quenched by addition of an equal volume of 5 mM13C5-citrulline in 30% TCA. Debris is separated by 5 minutes of microcentrifugation, and the supernatants are transferred to vials for mass spectroscopy. Ten μL of sample are injected into an Agilent 1100 series LC-MS under isocratic conditions with a mobile phase of 93% solvent A (1 ml trifluoroacetic acid in 1 L water): 7% solvent B (1 ml trifluoroacetic acid in IL of 1:9 water/acetonitrile). Peaks corresponding to citrulline [176.1 mass: charge ratio (m/z)] and 13C5-citrulline (181.1 m/z) are quantitated, and their ratios are compared to ratios obtained for a standard curve of citrulline run with each assay. Samples are normalized to either total liver tissue or to protein concentration determined using a Bio-Rad protein assay kit (Bio-Rad, Hercules, CA). Other assays, which do not require liver biopsy, may also be used. One such assay is a plasma amino acid assays in which the ratio of glutamine and citrulline is assessed and if glutamine is high (>800 microliters/liter) and citrulline low (e.g., single digits), a urea cycle defect is suspected. Plasma ammonia levels can be measured and a concentration of about 100 micromoles per liter is indicative of OTCD. Blood gases can be assessed if a patient is hyperventilating: respiratory alkalosis is frequent in OTCD. Orotic acid in urine, e.g., greater than about 20 micromoles per millimole creatine is indicative of OTCD, as is elevated urinary orotate after allopurinol challenge test. Diagnostic criteria for OTCD have been set forth in Tuchman et al, 2008, Urea Cycle Disorders Consortium (UCDC) of the Rare Disease Clinical Research Network (RDCRN). Tuchman M, et al., Consortium of the Rare Diseases Clinical Research Network. Cross-sectional multicenter study of patients with urea cycle disorders in the United States. Mol Genet Metab. 2008:94:397-402, which is incorporated by reference herein. See, also, http://www.ncbi.nlm.nih.gov/books/NBK154378/, which provides a discussion of the present standard of care for OTCD.

In certain embodiments, a nuclease expression cassette, non-viral vector, viral vector (e.g., rAAV), or any of the same in a pharmaceutical composition, as described herein is administrable for gene editing in a patient. In certain embodiments, the method is useful for non-embryonic gene editing. In certain embodiments, the patient is an infant (e.g., birth to about 9 months). In certain embodiments, the patient is older than an infant, e.g., 12 months or older.

As used herein. “a.” “an.” or “the” can mean one or more than one. For example. “a” cell can mean a single cell or a multiplicity of cells.

As used herein, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g. D M Mccarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535:7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same expression cassette or host cell, but which is present in a non-natural state, e.g., a different copy number, or under the control of different regulatory elements.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene.

As used herein, the term “host cell” may refer to the packaging cell line in which a vector (e.g., a recombinant AAV) is produced from a production plasmid. In the alternative, the term “host cell” may refer to any target cell in which expression of the transgene is desired. Thus, a “host cell,” refers to a prokaryotic or eukaryotic cell that contains a exogenous or heterologous nucleic acid sequence that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. In certain embodiments herein, the term “host cell” refers to cultures of cells of various mammalian species for in vitro assessment of the compositions described herein. In other embodiments herein, the term “host cell” refers to the cells employed to generate and package the viral vector or recombinant virus. Still in other embodiment, the term “host cell” is intended to reference the target cells of the subject being treated in vivo for the diseases or conditions as described herein. In certain embodiments, the term “host cell” is a liver cell or hepatocyte.

A “subject” is a mammal, e.g., a human, mouse, rat, guinea pig, dog, cat, horse, cow, pig, or non-human primate, such as a monkey, chimpanzee, baboon or gorilla. A patient refers to a human. A veterinary subject refers to a non-human mammal. In certain embodiments, the subject does not have a defect in their PCSK9 gene.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity.” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™ a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”. “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

As used herein, the term “about” refers to a variant of +10% from the reference integer and values therebetween. For example, “about” 40 base pairs, includes +4 (i.e., 36-44, which includes the integers 36, 37, 38, 39, 40, 41, 42, 43, 44). For other values, particularly when reference is to a percentage (e.g., 90% identity, about 10% variance, or about 36% mismatches), the term “about” is inclusive of all values within the range including both the integer and fractions.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example. 1, 2, 2.7, 3, 4, 5, 5.3, and 6. This applies regardless of the breadth of the range.

As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

Unless defined otherwise in this specification, technical and scientific terms used herein have the same meaning as commonly understood by one of ordinary skill in the art and by reference to published texts, which provide one skilled in the art with a general guide to many of the terms used in the present application.

### SPECIFIC EMBODIMENTS

1. A method of treating hemophilia B in an adolescent subject, the method comprising co-administering to the subject having hemophilia B:
   (a) a gene editing AAV vector comprising a sequence encoding a nuclease and regulatory sequences that direct expression of the nuclease in a target cell comprising a PCSK9 gene; and
   (b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.
2. A method of treating hemophilia B in an adult subject, the method comprising co-administering to the subject having hemophilia B:
   (a) a gene editing AAV vector comprising a sequence encoding a nuclease and regulatory sequences that direct expression of the nuclease in a target cell comprising a PCSK9 gene; and
   (b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.
3. The method of embodiment I or embodiment 2, wherein the ratio of gene editing vector to donor vector is about 1:3.
4. The method of any one of embodiments 1 to 3, wherein the donor AAV vector and/or gene editing AAV vector comprises an AAVhu37 capsid.
5. The method of any one of embodiments 1 to 4, wherein the donor AAV vector and/or gene therapy vector comprises an AAVrh79 capsid.
6. The method of any one of embodiments 1 to 5, wherein the gene editing AAV vector of (a) is suspended in a vehicle for injection at a concentration of about 2×10¹¹ GC/mL to about 2×10¹² GC/mL.
7. The method of any one of embodiments 1 to 6, wherein the AAV donor vector of (b) is suspended in a vehicle for injection at a concentration of about 2×10¹² GC/mL to about 1×10¹³ GC/mL.
8. A method for treating hemophilia B in an adolescent subject in need thereof, the method comprising co-administering to the subject having hemophilia B:
   (a) a lipid nanoparticle (LNP) comprising a mRNA sequence encoding a nuclease; and
   (b) a donor AAV vector comprising a factor IX transgene and regulatory sequences which direct its expression in the target cell, the donor vector further comprising a homology-directed recombination (HDR) arms 5′ and 3′ to the transgene.
9. A method for treating hemophilia B in an adult subject in need thereof, the method comprising co-administering to the subject having hemophilia B:
   (a) a lipid nanoparticle (LNP) comprising a mRNA sequence encoding a nuclease; and
   (b) a donor AAV vector comprising a factor IX transgene and regulatory sequences which direct its expression in the target cell, the donor vector further comprising a homology-directed recombination (HDR) arms 5′ and 3′ to the transgene.
10. The method of any one of embodiments 1 to 9, wherein the donor AAV vector comprises an AAVhu37 capsid.
11. The method of any one of embodiments 1 to 9, wherein the donor AAV vector comprises an AAVrh79 capsid.
12. The method of any one of embodiments 1 to 11, wherein the nuclease targets the PCSK9 gene.
13. The method of embodiment 12, wherein the nuclease targets PCSK9 exon 7.
14. The method of any one of embodiments 1 to 13, wherein the nuclease is a meganuclease specific for PCSK9.
15. The method of embodiment 14, wherein the meganuclease is the ARCUS meganuclease.
16. The method of any one of embodiments 1 to 13, wherein the nuclease is a Cas9 nuclease and wherein said method further comprises administering an sgRNA.
17. The method of embodiment 16, wherein said LNP comprises the sgRNA.
18. The method of embodiment 16 or embodiment 17, wherein the Cas9 nuclease is flanked by nuclear localization signals.
19. The method of any one of embodiments 16 to 18, wherein the sgRNA comprises at least 20 nucleotides which specifically bind to a target site in the PCSK9 gene, said target site being 5′ to a protospacer-adjacent motif (PAM) that is specifically recognized by the Cas9.
20. The method of any one of embodiments 8 to 13 or 16 to 19, further comprising an RNA polymerase promoter.
21. The method of embodiment 20, wherein the RNA polymerase promoter is the U6 promoter.
22. The method of embodiment 21, wherein the U6 promoter is located 5′ of the sgRNA.
23. The method of any one of embodiments 16 to 22, wherein the sgRNA is 100% complementary to the target site sequence.
24. The method of any one of embodiments 16 to 22, wherein the sgRNA is less than 100% complementary to the target site sequence.
25. The method of any one of embodiments 16 to 24, wherein Cas9 is selected from Staphylococcus aureus or Streptococcus pyogenes Cas9.
26. The method of any one of embodiments 1 to 25, wherein the nuclease is under the control of a tissue-specific promoter.
27. The method of any one of embodiments 1 to 25, wherein the nuclease is under the control of a constitutive promoter.
28. The method of embodiment 26, wherein the nuclease is under the control of a liver-specific promoter, optionally a human thyroxin-binding globulin (TBG) promoter, or hybrid liver promoter (HLP).
29. The method of any preceding embodiment, wherein the gene editing AAV vector of (a) and the donor vector of (b) are delivered essentially simultaneously via the same route.
30. The method of any preceding embodiment, further comprising administering an anti-CD20 ligand, optionally rituximab.
31. A dual component system useful for treating hemophilia B in an adolescent subject, the system comprising:
   (a) a gene editing AAV vector comprising a sequence encoding a nuclease and regulatory sequences that direct expression of the nuclease in a target cell comprising a PCSK9 gene; and
   (b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.
32. A dual component system useful for treating hemophilia B in an adult subject, the method system comprising:
   (a) a gene editing AAV vector comprising a sequence encoding a nuclease and regulatory sequences that direct expression of the nuclease in a target cell comprising a PCSK9 gene; and
   (b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.
33. The dual component system of embodiment 31 or embodiment 32, wherein the ratio of gene editing vector to donor vector is about 1:3.
34. The dual component system of any one of embodiments 31 to 33, wherein the donor AAV vector and/or gene editing AAV vector comprises an AAVhu37 capsid.
35. The dual component system of any one of embodiments 31 to 34, wherein the donor AAV vector and/or gene therapy vector comprises an AAVrh79 capsid.
36. The dual component system of any one of embodiments 31 to 35, wherein the gene editing AAV vector of (a) is suspended in a vehicle for injection at a concentration of about 2×10¹¹ GC/mL to about 2×10¹² GC/mL.
37. The dual component system of any one of embodiments 31 to 36, wherein the AAV donor vector of (b) is suspended in a vehicle for injection at a concentration of about 2×10¹² GC/mL to about 1×10¹³ GC/mL.
38. A dual component system useful for treating hemophilia B in an adolescent subject in need thereof, the system comprising:
   (a) a lipid nanoparticle (LNP) comprising a mRNA sequence encoding a nuclease; and
   (b) a donor AAV vector comprising a factor IX transgene and regulatory sequences which direct its expression in the target cell, the donor vector further comprising a homology-directed recombination (HDR) arms 5′ and 3′ to the transgene.
39. A method for treating hemophilia B in an adult subject in need thereof, the method comprising co-administering to the subject having hemophilia B:
   (a) a lipid nanoparticle (LNP) comprising a mRNA sequence encoding a nuclease; and
   (b) a donor AAV vector comprising a factor IX transgene and regulatory sequences which direct its expression in the target cell, the donor vector further comprising a homology-directed recombination (HDR) arms 5′ and 3′ to the transgene.
40. The method of any one of embodiments 31 to 39, wherein the donor AAV vector comprises an AAVhu37 capsid.
41. The method of any one of embodiments 31 to 39, wherein the donor AAV vector comprises an AAVrh79 capsid.
42. The method of any one of embodiments 31 to 41, wherein the nuclease targets the PCSK9 gene.
43. The method of embodiment 42, wherein the nuclease targets PCSK9 exon 7.
44. The method of any one of embodiments 31 to 43, wherein the nuclease is a meganuclease specific for PCSK9.
45. The method of embodiment 44, wherein the meganuclease is the ARCUS meganuclease.
46. The method of any one of embodiments 31 to 43, wherein the nuclease is a Cas9 nuclease and wherein said method further comprises administering an sgRNA.
47. The method of embodiment 46, wherein said LNP comprises the sgRNA.
48. The method of embodiment 46 or embodiment 47, wherein the Cas9 nuclease is flanked by nuclear localization signals.
49. The method of any one of embodiments 46 to 48, wherein the sgRNA comprises at least 20 nucleotides which specifically bind to a target site in the PCSK9 gene, said target site being 5′ to a protospacer-adjacent motif (PAM) that is specifically recognized by the Cas9.
50. The method of any one of embodiments 38 to 43 or 46 to 49, further comprising an RNA polymerase promoter.
51. The method of embodiment 50, wherein the RNA polymerase promoter is the U6 promoter.
52. The method of embodiment 51, wherein the U6 promoter is located 5′ of the sgRNA.
53. The method of any one of embodiments 46 to 52, wherein the sgRNA is 100% complementary to the target site sequence.
54. The method of any one of embodiments 46 to 52, wherein the sgRNA is less than 100% complementary to the target site sequence.
55. The method of any one of embodiments 46 to 54, wherein Cas9 is selected from Staphylococcus aureus or Streptococcus pyogenes Cas9.
56. The method of any one of embodiments 31 to 55, wherein the nuclease is under the control of a tissue-specific promoter.
57. The method of any one of embodiments 31 to 55, wherein the nuclease is under the control of a constitutive promoter.
58. The method of embodiment 26, wherein the nuclease is under the control of a liver-specific promoter, optionally a human thyroxin-binding globulin (TBG) promoter, or hybrid liver promoter (HLP).
59. The method of any preceding embodiment, wherein the gene editing AAV vector of (a) and the donor vector of (b) are delivered essentially simultaneously via the same route.

### EXAMPLES

Ornithine transcarbamylase (OTC) deficiency is an X-linked urea cycle disorder associated with high mortality. Although a promising treatment for late-onset OTC deficiency, adeno-associated virus (AAV) neonatal gene therapy would only provide short-term therapeutic effects as the non-integrated genome gets lost during hepatocyte proliferation. Nuclease-mediated, site-specific integration of an OTC mini gene cassette in a safe harbor in the genome would provide long-term therapeutic benefits to patients with OTC deficiency. One of the safe harbors for gene targeting is the PCSK9 gene, such as the exon 7 region. The nucleases could be an engineered meganuclease targeting PCSK9 (ARCUS2) or CRISPR/Cas9 with specific sgRNA targeting PCSK9. The donor vector contains a mini gene including a liver-specific promoter such as a TBG promoter, a codon optimized hOTC coding sequence, and a poly A sequence. Both the nuclease and the donor template could be delivered by AAV vectors (dual AAV vector system). Demonstrated persistent transgene expression and efficient gene targeting in 12% of hepatocytes at 12 weeks after a single intravenous injection of the dual AAV vectors in a newborn nonhuman primate (NHP). The mini gene in the donor vector is flanked with homolog-directed recombination (HDR) arms.

First time demonstration in NHPs of in vivo nuclease-mediated gene targeting to the PCSK9 locus to express a therapeutic protein following a single injection of the dual AAV vectors as newborns or infants. Composition of the donor vector containing the OTC mini gene for gene targeting to the human/NHP PCSK9 locus has not been tested in the clinics for the treatment of OTC deficiency. We will test the hOTC donor vectors in newborn NHPs for gene targeting efficiency and in newborn transgenic OTC deficient mice for efficacy.

Many metabolic diseases require early intervention and therapy; however. AAV-mediated neonatal gene therapy is unstable due to fast liver proliferation in the neonatal stage and non-integrative nature of the AAV vector. Targeted integration of a therapeutic mini gene cassette in a safe harbor would persistently express the therapeutic gene on the genome level and the therapeutic effects would be maintained through cell division. For many metabolic diseases such as OTC deficiency, sufficient transduction efficiency in liver needs to be achieved for clinical benefits.

We describe a genome editing approach for the treatment of ornithine transcarbamylase deficiency (OTCD) which can cause lethal episodes of hyperammonemia in infancy. The goal of genome editing is for the therapeutic effect to be durable and achieved in all OTCD patients independent of their mutation. We propose to accomplish this by treating surviving newborns with two AAV vectors: one to deliver a nuclease to create a double stranded break in a safe-harbor stie and the second to deliver an OTC minigene for knock-in into this site. Our assumption is that dividing hepatocytes of the newborn liver will be conducive to efficient knock-in of the OTC gene and will eliminate, through dilution, the non-integrated input vector genomes. We decided to use the PCSK9 gene as a safe-harbor site and a meganuclease, called ARCUS to target it, based on our previous work in adult macaques which showed safe, efficient and stable reductions of PCSK9 following AAV delivery ARCUS. Our initial studies of genome editing for OTCD were performed in an OTC deficient mouse rendered susceptible to the PCSK9 ARCUS nuclease through germ line modification of exon 7 of the endogenous PCSK9 gene. Injection of the two vectors into newborn mice resulted in efficient knock-in of the human OTC minigene and protection to lethal hyperammonemia when challenged with a high protein diet. In preparation for clinical studies, we evaluated key safety and efficacy parameters in newborn and infant macaques. A total of 24 animals were treated with AAV vectors with analyses to include examination of liver biopsies at 3 and 12 months. In these studies we evaluated the impact of the following parameters on editing efficiency and toxicity: transgene (human factor IX and human OTC), promoters driving ARCUS, Clade E capsids, length of donor flanking the transgene and age of the macaque at time of dosing. We report here preliminary data of 16/24 animals that includes, at a minimum, 3 month biopsy results. We found the injection of AAV vectors was quite safe with no evidence of transaminase elevations or liver histopathology in any ARCUS treated animals. The key measure of efficacy in the primate model is transduction efficiency measured by in situ hybridization and immunostaining to detect cells expressing the human OTC mRNA and protein, respectively. The highest and most consistent results were obtained with vectors using a novel clade E capsid driving ARCUS with a TBG promoter in the first vector and using 500 bp flanking homology arms on the donor vector. With this combination we achieved 10.0±6.4% (N=6) transduction which is higher than the threshold we believe can provide substantial benefit to patients which is ˜5% OTC expressing cells. Preliminary data suggests that the level of editing is stable over one year and that efficient targeted insertion can be achieved when injected into macaques up to 3 months of age. Molecular analyses of the PCSK9 target locus suggested the vast majority of the knocked-in of vector genome were through non-homologous end joining (NHEJ) rather than homology-directed repair (HDR). In summary, the substantial unmet need of the neonatal form of OTCD warrants consideration of experimental therapies such as genome editing such as that described in this report.

### Example 1—Materials and Methods

### Materials and Methods

AAV vectors were constructed according to previously established procedures and manufacturer's instructions. The AAVhu37 capsid was used for the experiments as described herein, where indicated.

All animal procedures were performed in accordance with protocols approved by the Institutional Animal Care and Use Committee of the University of Pennsylvania.

### Example 2—Pilot Study: HFIX Mini Gene Knock-In in Pcsk9 Locus by ARCUS2 or SACAS9 in Newborn NHPS

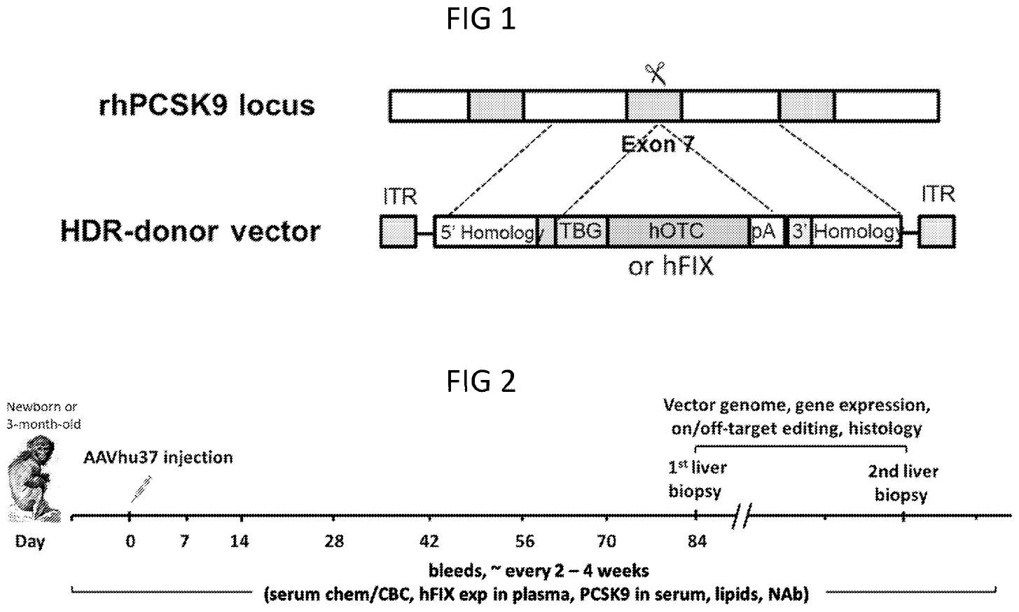

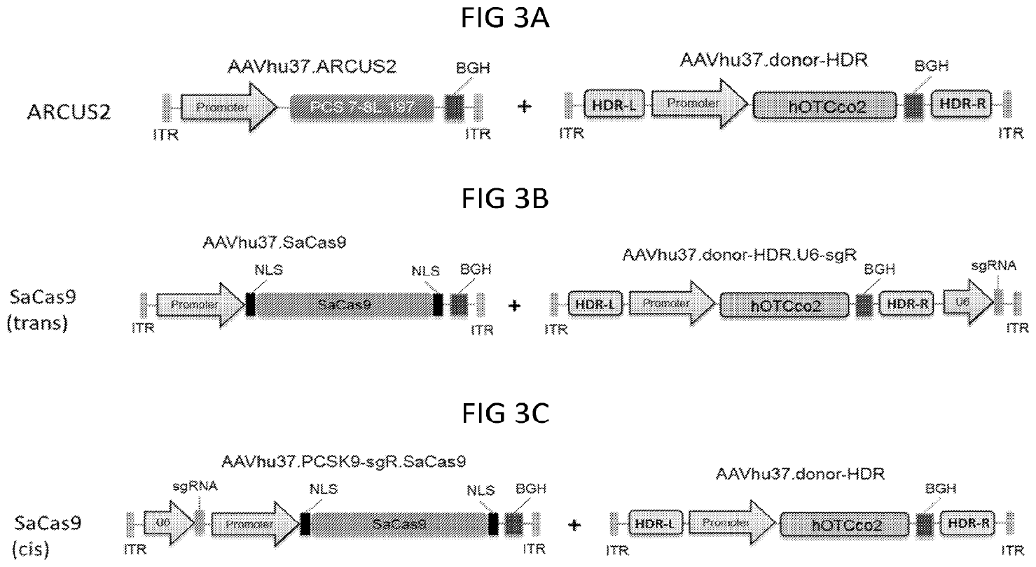

In this study, we evaluated on-target (PSK9) SaCas9- or ARCUS-mediated gene editing and hFIX or OTC mini-gene knock in efficiency in newborn non-human primates (NHPs). FIG. 1 shows a schematic representation of the rhPCSK9 locus showing the donor splice site within exon 7, and a HDR donor vector comprising donor template of interest, e.g., hFIX, hOTC. Furthermore, FIGS. 3A to 3C show a schematic representation for a dual AAV vector system for SaCas9- or ARCUS-mediated gene correction. FIG. 3A shows a schematic representation for a dual AAVhu37 vector system for ARCUS2-mediated gene correction, wherein the AAVhu37-donor vector comprises an hOTC donor template sequence. FIG. 3B shows a schematic representation for a dual AA Vhu37 vector system for Sa-Cas9-mediated gene correction (trans: AAVhu37-SaCas9), wherein the AAV.hu37.shRNA-donor vector comprises an hOTC donor template sequence. FIG. 3C shows a schematic representation for a dual AAVhu37 vector system for Sa-Cas9-mediated gene correction (cis: AAVhu37.PCSK9-sgRN.SaCas9), wherein the AAV.hu37-donor vector comprises an hOTC donor template sequence.

The above described dual AAVhu37 vectors comprising gene editing nuclease and donor templates, were used in newborn NHP to examine the hFIX mini-gene knock-in in PCSK9 locus as mediated by either SaCas9 or ARCUS2. Gene editing AAVhu37 vector was delivered at a dose of 1×10¹³ GC/kg, and donor template AA Vhu37 vector was delivered at 3×10¹³ GC/kg. Overall, there were three treatment NHP groups: 1) AAVhu37.EGFP and AAVhu37.Donor-HDR-hFIX.U6.sgR: 2) AA Vhu37.ARCUS2 and AA Vhu37.Donor-HDR-hFIX: 3) AAVhu37.SaCas9 and AAVhu37.Donor-HDR-hFIX.U6.sgR. FIG. 2 shows a timeline for a pilot study comprising an hFIX mini-gene knock-in in PCSK9 locus by ARCUS2 or SaCas9 in newborn NHPs. In this study, NHPs were injected at day 0, blood samples were collected at every 2-4 weeks (to examine serum chemistry, hFIX expression in plasma, PCSK9 levels in serum, LDL levels and neutralizing antibodies (NAb) levels), first liver biopsy was performed at day 84 (to examine vector genome levels, gene expression levels, on- an off-target editing, and histology).

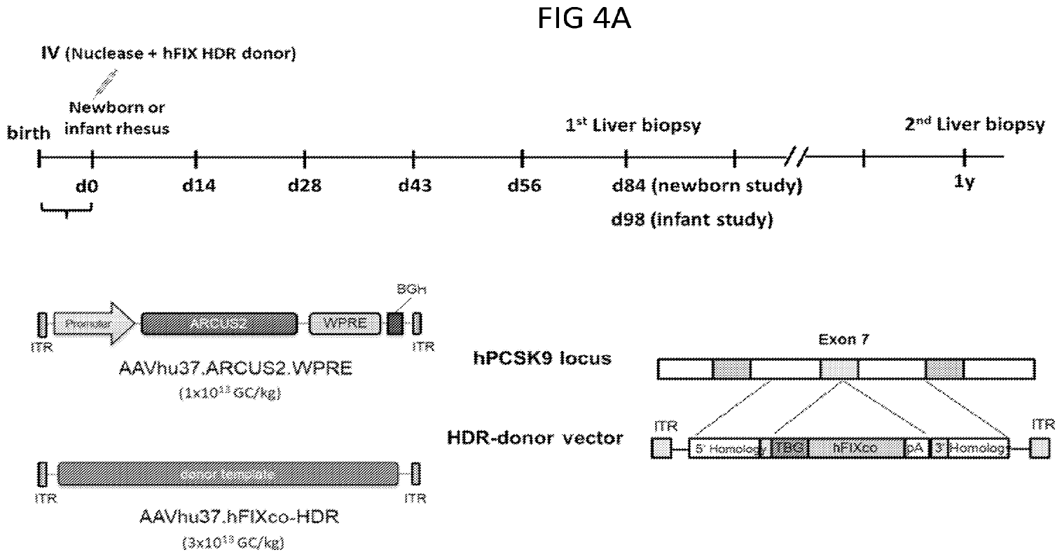

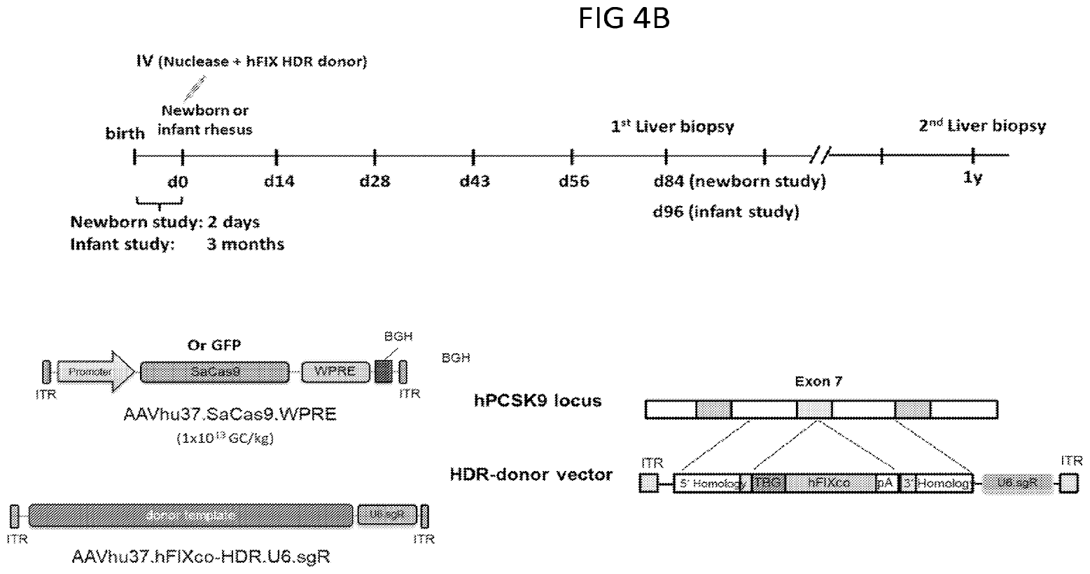

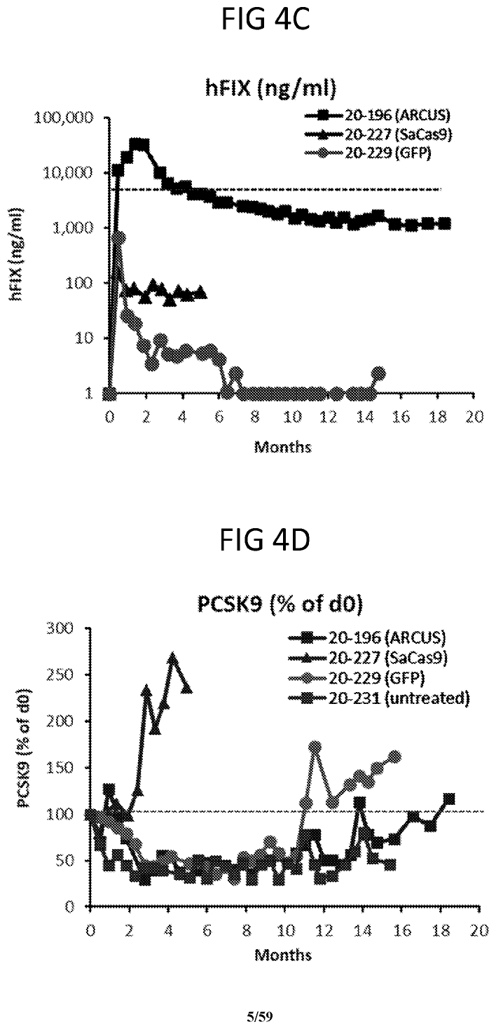

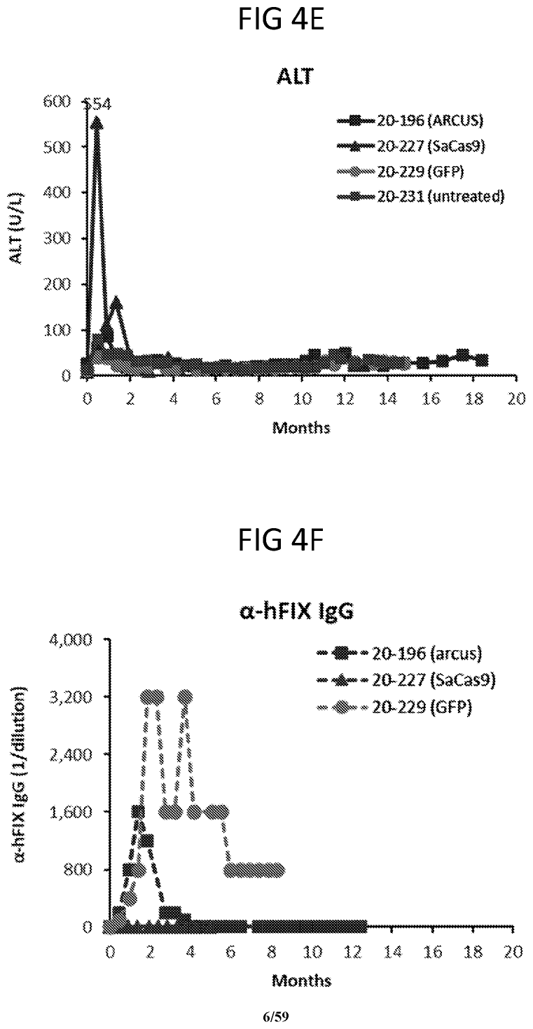

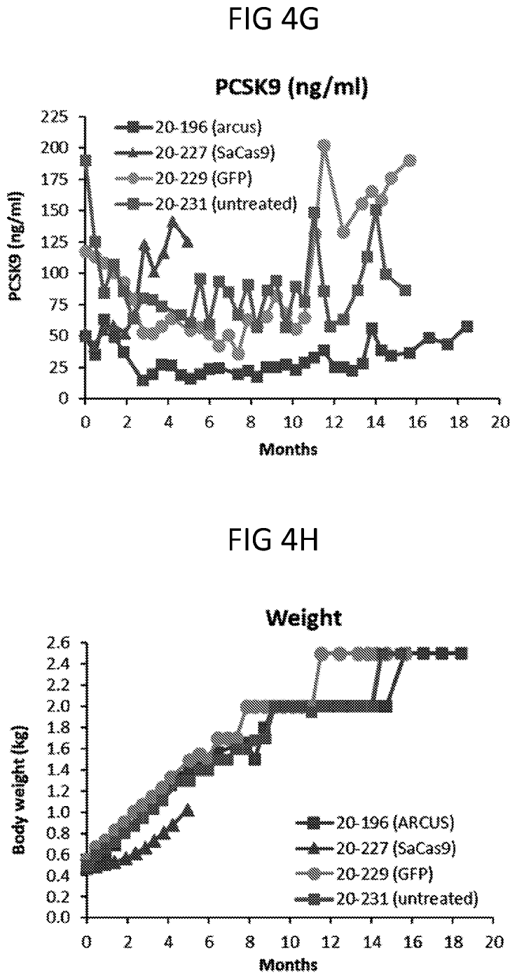

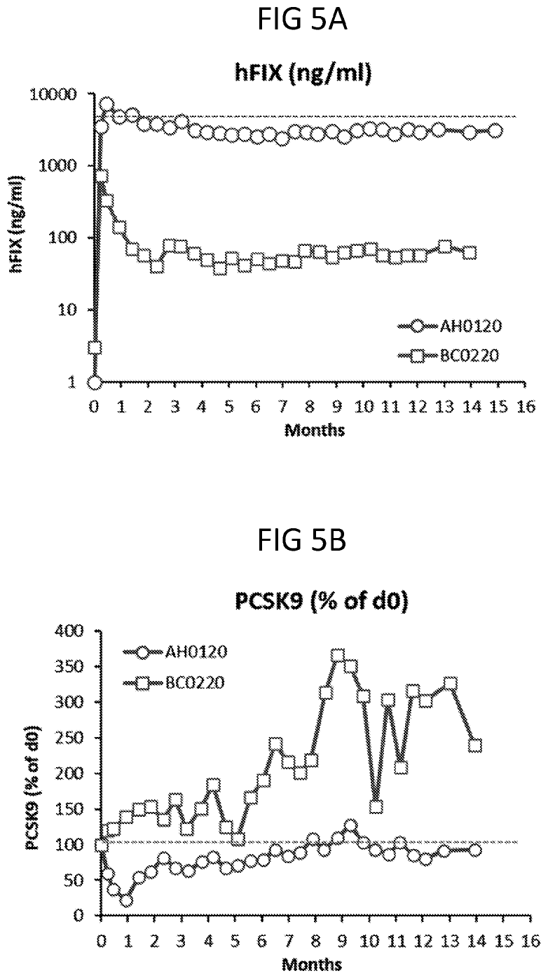

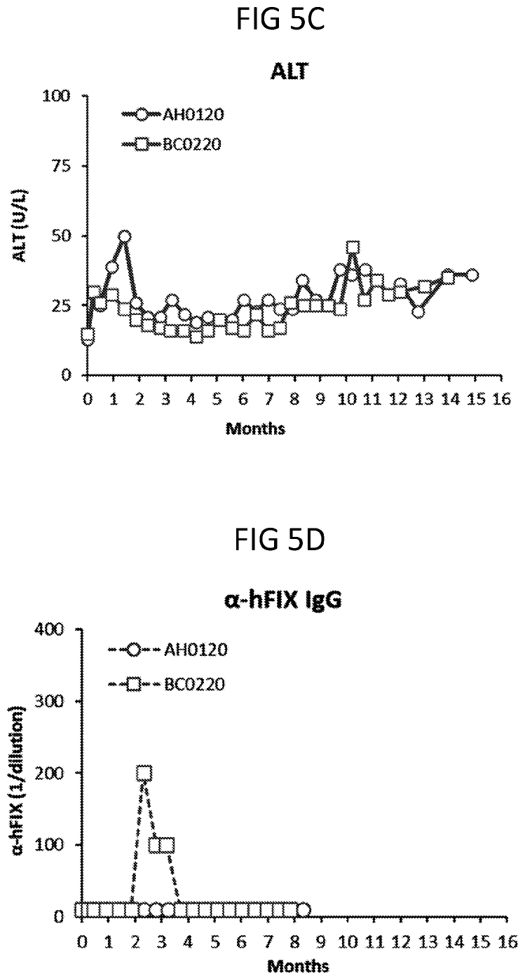

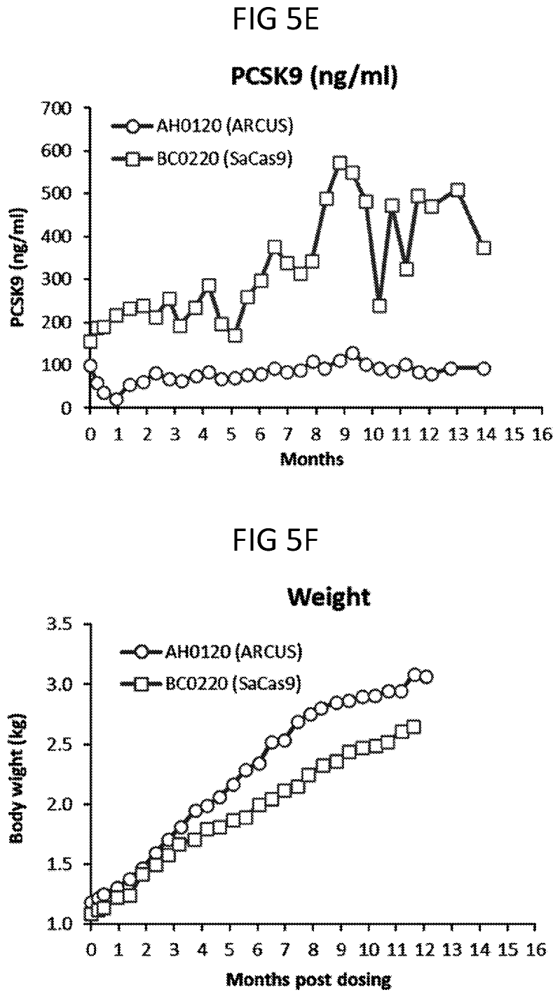

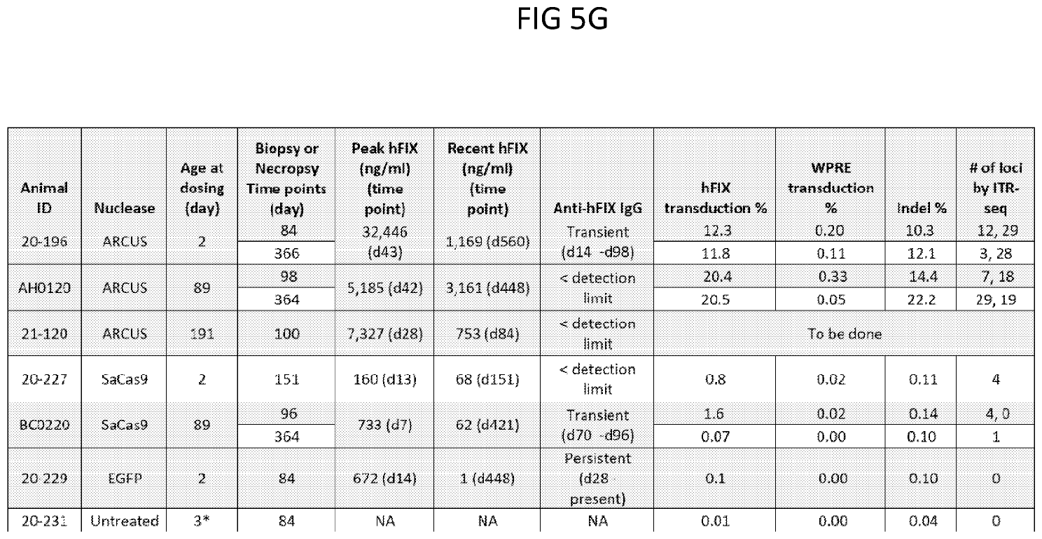

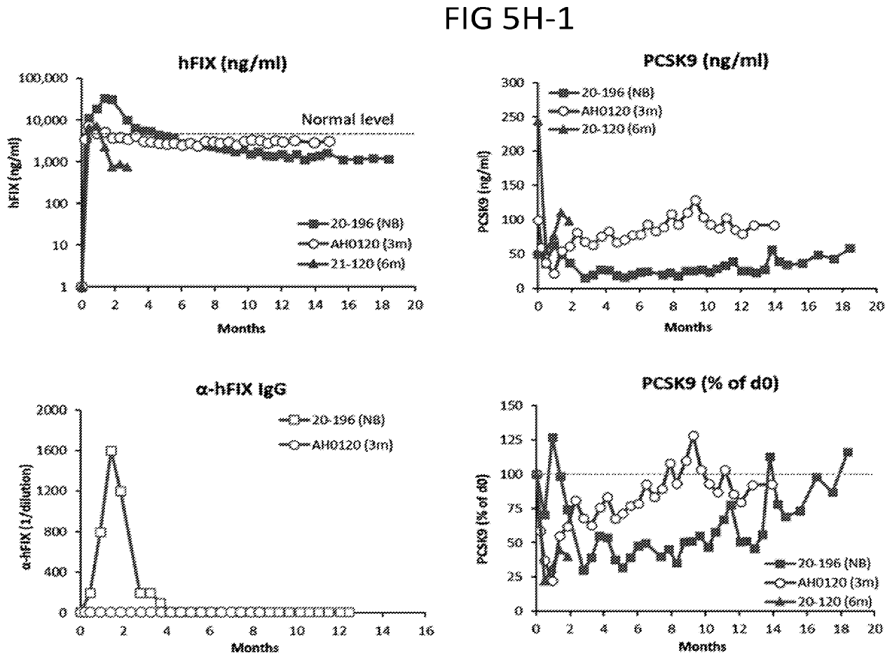

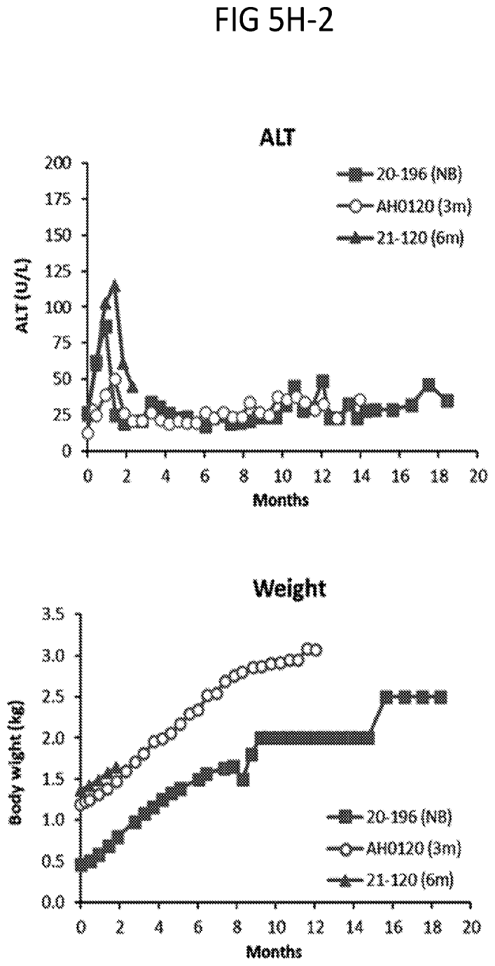

An in vivo test of nuclease-mediated gene targeting was performed in newborn and infant NHPs. Animals were administered with 1×10¹³ GC/kg of AAVhu37.ARCUS2.WPRE and 3×10¹³ GC/kg of AAVhu37.hFIXco-HDR or 1×10¹³ GC/kg of AAVhu37.SaCas9.WPRE and 3×10¹³ GC/kg of AAVhu37.hFIXco-HDR.U6.sgR or 1×10¹³ GC/kg of AAVhu37.GFP.WPRE and 3×10¹³ GC/kg of AAVhu37.hFIXco-HDR.U6.sgR, as shown in FIGS. 4A. 4B, and 5G. FIG. 4C shows hFIX levels at the indicated timepoints from day 0 to 13 months post treatment (plotted as ng/ml). FIG. 4D shows PCSK9 levels at the indicated timepoints from day 0 to 12 months post treatment (plotted as percentage of baseline at day 0). FIG. 4E shows ALT (Alanine Aminotransferase) levels at the indicated timepoints from day 0 to day 196 post treatment (plotted as U/L). FIG. 4F shows anti-FIX IgG levels at the indicated timepoints from day 0 to day 196 post treatment (plotted as dilution factor, 1/dilution). FIG. 4G shows PCSK9 levels at the indicated timepoints from day 0 to day 196 post treatment (plotted as ng/mL). FIG. 4H shows weight as measured at the indicated timepoints from day 0 to day 196 post treatment (plotted as g). FIG. 5A shows hFIX levels at the indicated timepoints (plotted as ng/ml) in infant NHPs. FIG. 5B shows PCSK9 levels at the indicated timepoints (plotted as percentage of baseline at day 0) in infant NHPs. FIG. 5C shows ALT (Alanine Aminotransferase) levels at the indicated timepoints (plotted as U/L) in infant NHPs. FIG. 5D shows anti-FIX IgG levels at the indicated timepoints (plotted as dilution factor, 1/dilution) in infant NHPs. FIG. 5E shows PCSK9 levels at the indicated timepoints (plotted as ng/mL) in infant NHPs. FIG. 5F shows weight as measured at the indicated timepoints (plotted as g) in infant NHPs. FIG. 5G is a summary table showing data from the experiment described in FIGS. 4A-5G. FIG. 5H shows a comparison of various data between newborn and infant NHPs tested.

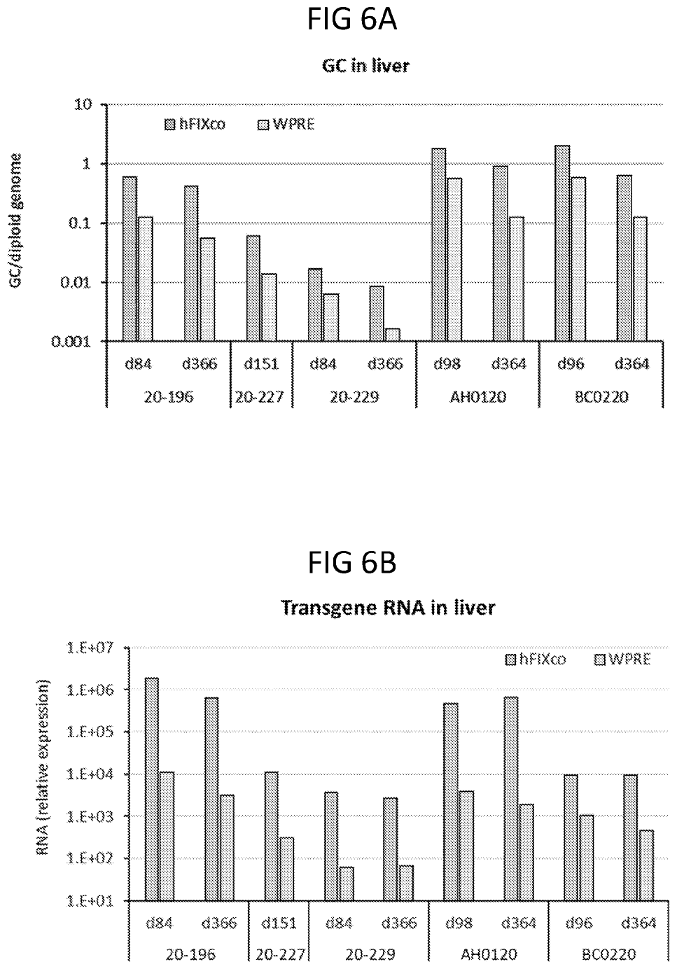

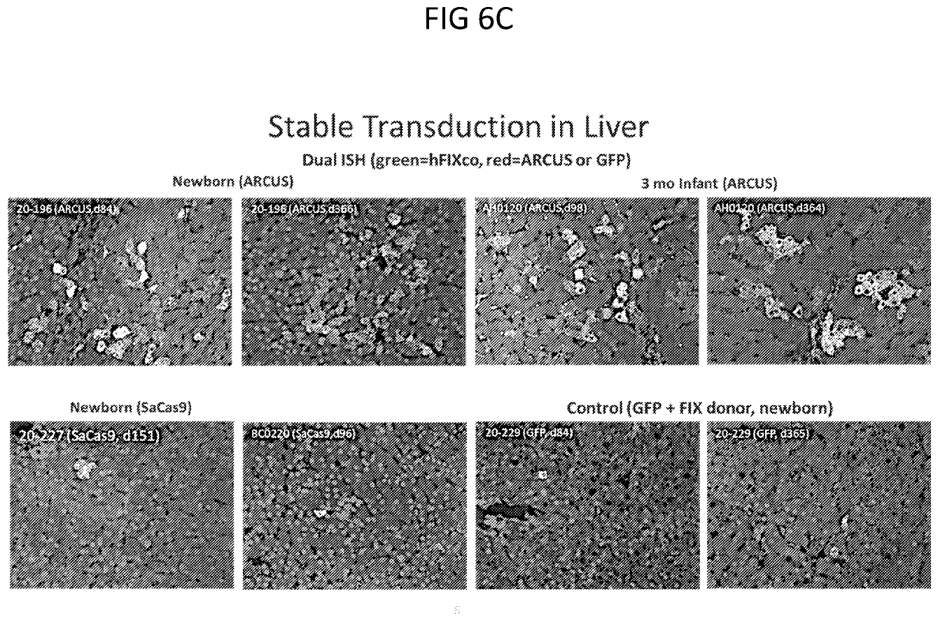

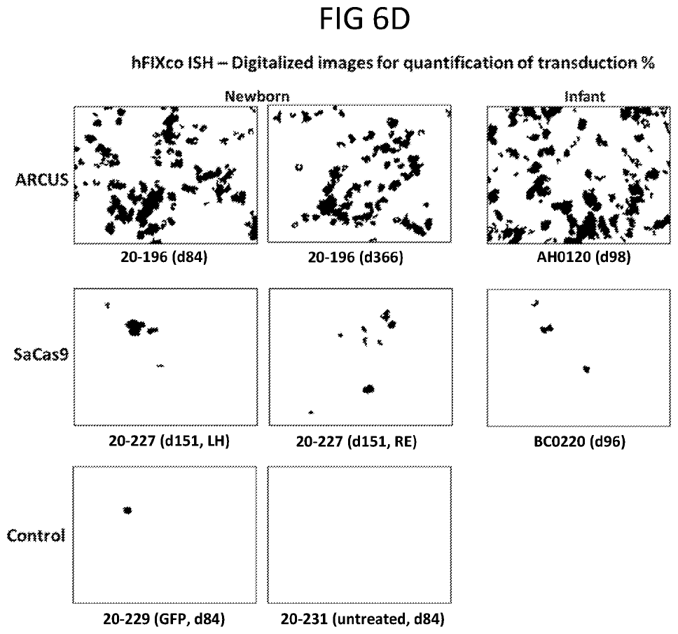

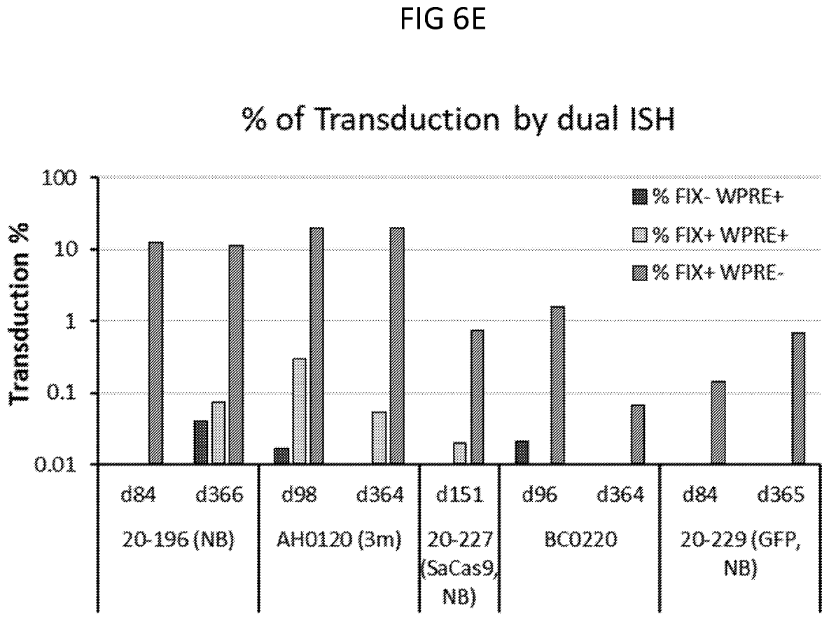

FIGS. 6A to 6E show vector transduction (GC) and transgene expression in liver biopsies samples collected at days shown post treatment in the NHPs. 6A shows vector transduction levels in liver biopsies samples, plotted as AAV genome copies (GC) per diploid cell. FIG. 6B shows relative expression of transgene RNA in liver biopsies samples. FIG. 6C shows dual in situ hybridization (ISH) using specific probes to detect FIX and ARCUS in liver biopsies. FIG. 6D shows digitized ISH images used for quantification of transduction. FIG. 6E shows transduction efficiency of FIX transgene as quantified by ISH, and plotted as percent transduction.

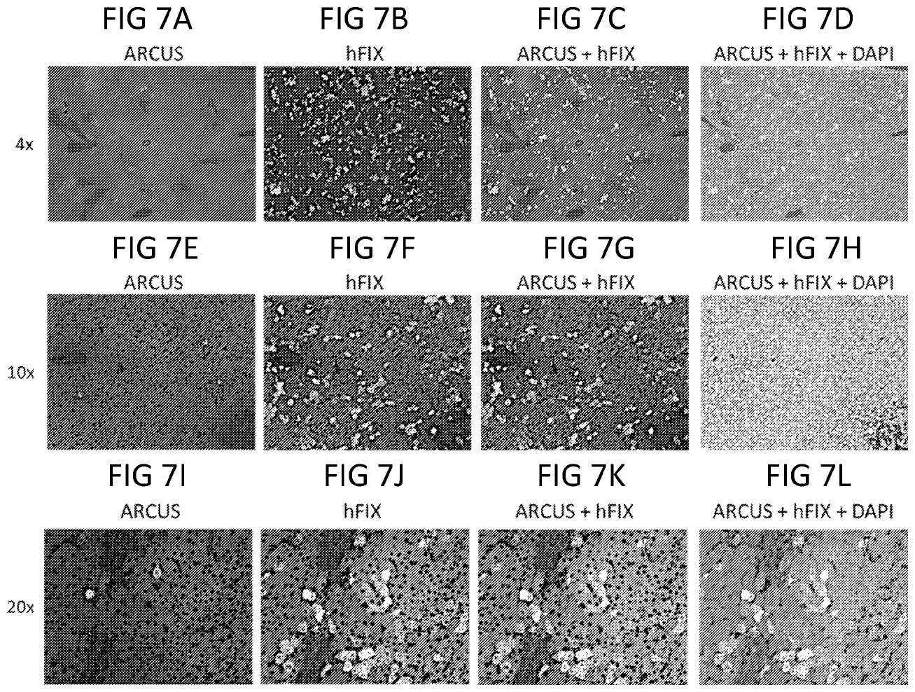

FIGS. 7A to 7L show dual in situ hybridization (ISH) using specific probes to detect FIX and ARCUS in liver biopsies collected at 84 days post treatment in NHPs: showed at various magnification views (NHPs treated with AAVhu37.ARCUS2 and AAVhu37.Donor-HDR-hFIX). FIG. 7A shows ISH-detected ARCUS in liver biopsies, viewed at 4× magnification. FIG. 7B shows ISH-detected hFIX in liver biopsies, viewed at 4× magnification. FIG. 7C shows overlay image of ISH-detected ARCUS and hFIX, viewed at 4× magnification. FIG. 7D shows ISH-detected ARCUS and hFIX as an overlayed image with DAPI (staining for nuclei), viewed at 4× magnification. FIG. 7E shows ISH-detected ARCUS in liver biopsies, viewed at 10× magnification. FIG. 7F shows ISH-detected hFIX in liver biopsies, viewed at 10× magnification. FIG. 7G shows overlay image of ISH-detected ARCUS and hFIX, viewed at 10× magnification. FIG. 7H shows ISH-detected ARCUS and hFIX as an overlayed image with DAPI (staining for nuclei), viewed at 10× magnification. FIG. 7I shows ISH-detected ARCUS expression in liver biopsies, viewed at 20× magnification. FIG. 7J shows ISH-detected hFIX in liver biopsies, viewed at 20× magnification. FIG. 7K shows overlay image of ISH-detected ARCUS and hFIX, viewed at 20× magnification. FIG. 7L shows ISH-detected ARCUS and hFIX as an overlayed image with DAPI (staining for nuclei), viewed at 20× magnification. Summary of vector transduction (GC/diploid genome) is shown in table 1 below.

TABLE 1

| | GC/diploid genome |
|---|---|
| hFIX | 0.63 |
| ARCUS | 0.13 |
| Ratio (FIX/ARCUS) | 4.8 |
| Ratio of vector dose | 3.0 |
| (FIX/ARCUS) | |

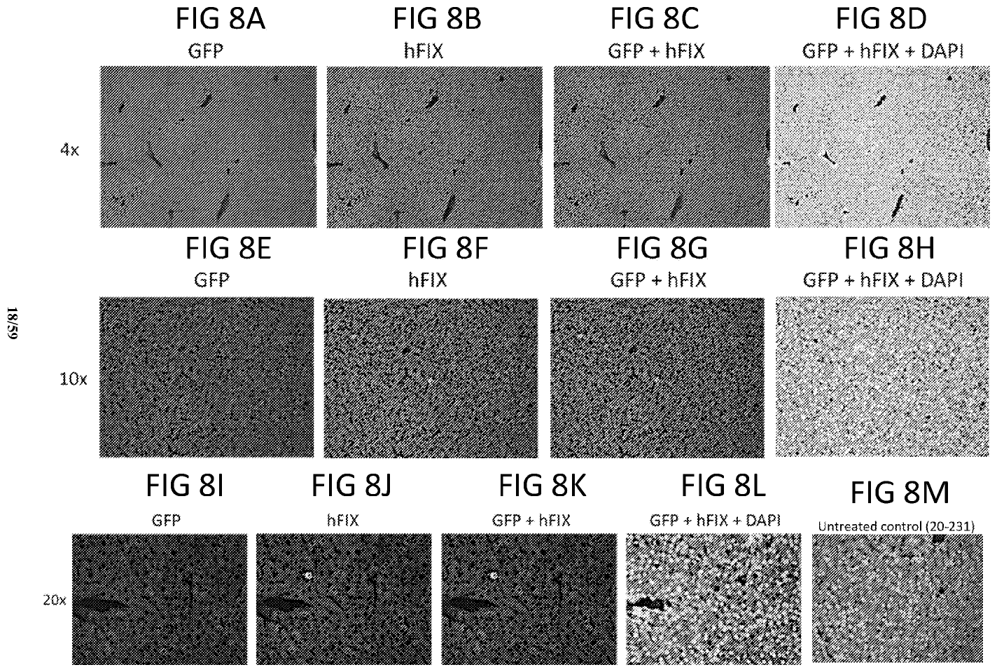

FIGS. 8A to 8M show dual in situ hybridization (ISH) using specific probes to detect FIX and ARCUS in liver biopsies collected at 84 days post treatment in NHPs; showed at various magnification views (NHPs treated with AAVhu37.EGFP and AAVhu37.Donor-HDR-hFIX.U6.sgR). FIG. 8A shows ISH-detected GFP-WRPE in liver biopsies, viewed at 4× magnification. FIG. 8B shows ISH-detected hFIX in liver biopsies, viewed at 4× magnification. FIG. 8C shows overlay image of ISH-detected GFP-WRPE and hFIX, viewed at 4× magnification. FIG. 8D shows ISH-detected GFP-WRPE and hFIX as an overlayed image with DAPI (staining for nuclei), viewed at 4× magnification. FIG. 8E shows ISH-detected GFP-WRPE in liver biopsies, viewed at 10× magnification. FIG. 8F shows ISH-detected hFIX in liver biopsies, viewed at 10× magnification. FIG. 8G shows overlay image of ISH-detected GFP-WRPE and hFIX, viewed at 10× magnification. FIG. 8H shows ISH-detected GFP-WRPE and hFIX as an overlayed image with DAPI (staining for nuclei), viewed at 10× magnification. FIG. 8I shows ISH-detected GFP-WRPE expression in liver biopsies, viewed at 20× magnification. FIG. 8J shows ISH-detected hFIX in liver biopsies, viewed at 20× magnification. FIG. 8K shows overlay image of ISH-detected GFP-WRPE and hFIX, viewed at 20× magnification. FIG. 8L shows ISH-detected GFP-WRPE and hFIX as an overlayed image with DAPI (staining for nuclei), viewed at 20× magnification. FIG. 8M shows ISH-detected GFP-WRPE and hFIX as an overlayed image with DAPI (staining for nuclei), viewed at 20× magnification in an untreated control. Summary of vector transduction (GC/diploid genome) is shown in table 2 below.

TABLE 2

| | GC/diploid genome |
|---|---|
| hFIX | 0.017 |
| ARCUS | 0.006 |
| Ratio (FIX/ARCUS) | 2.7 |
| Ratio of vector dose | 3.0 |
| (FIX/ARCUS) | |

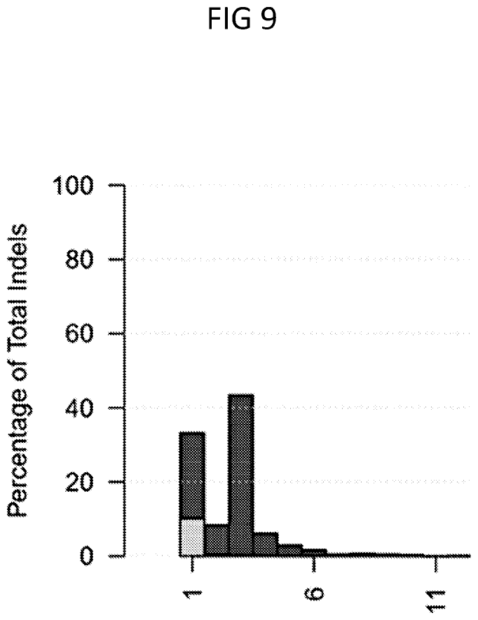

FIG. 9 shows ARCUS-mediated on-target editing in NHP treated with AAVhu37.ARCUS2 and AAVhu37.Donor-HDR-hFIX. At 84 days post treatment, liver biopsies samples were collected, and percentage of total indels in the target region present in was calculated. Furthermore, ARCUS-mediated on-target editing in NHP treated with AAVhu37.ARCUS2 and AAVhu37.Donor-HDR-hFIX. At 84 days post treatment, liver biopsies samples were collected, and frequencies of total indels in the target region present in was calculated, plotted as frequency of unique UMI OT reads relative to the target. Summary of indel as quantified by amplicon sequencing is shown in Table 3 below.

TABLE 3

| | 20-196 | 20-196 (NB, | AH0120 (3 m, | RA3567 |
|---|---|---|---|---|
| ID | (NB, d 84) | d 366) | d 98) | (ctl) |
| Ins % | 1.1 | 1.3 | 1.2 | 0.00 |
| Del % | 9.2 | 10.9 | 13.2 | 0.04 |
| Total | 10.2 | 12.1 | 14.4 | 0.04 |
| Indel % | | | | |
| HDR % | 2.8 | 2.0 | 1.2 | |
| (LMU- | | | | |
| seq) | | | | |

### Example 3—ARCUS2-Mediated HOTC Gene Targeting in Newborn NHPS

Newborn (1-16 days old) or infant (3-4 months old) rhesus macaques were used in non-GLP-compliant POC pharmacology studies. The M2PCSK9 meganuclease targets a 22-bp sequence present in the human and rhesus macaque PCSK9 gene. Thus, rhesus macaques can be used to evaluate on-target editing (pharmacology) and safety/toxicology. Furthermore, newborn and infant rhesus macaques have similar anatomical and physiological features as human infants and will allow for the use of the intended clinical ROA (IV). It is anticipated that the similarity in anatomy and ROA will result in representative vector distribution and transduction profiles, which will enable more accurate assessment of the pharmacology and toxicity of the test article, including on- and off-target editing, and clinical pathology, which is not possible in newborn mice.

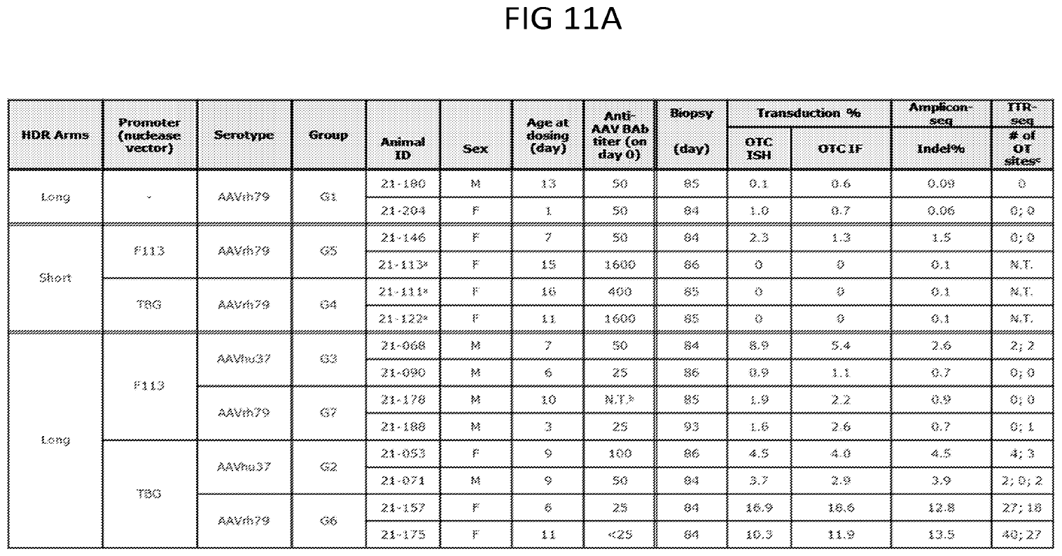

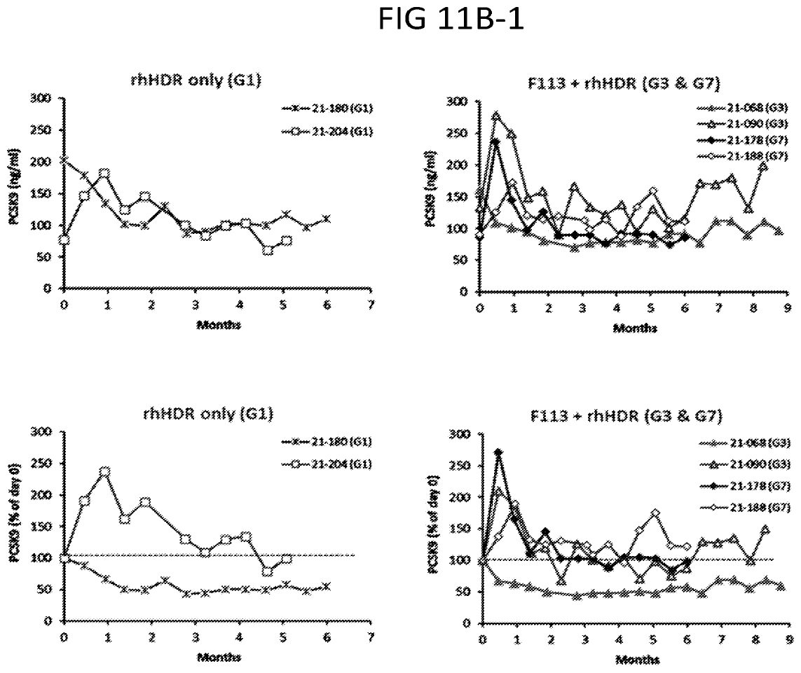

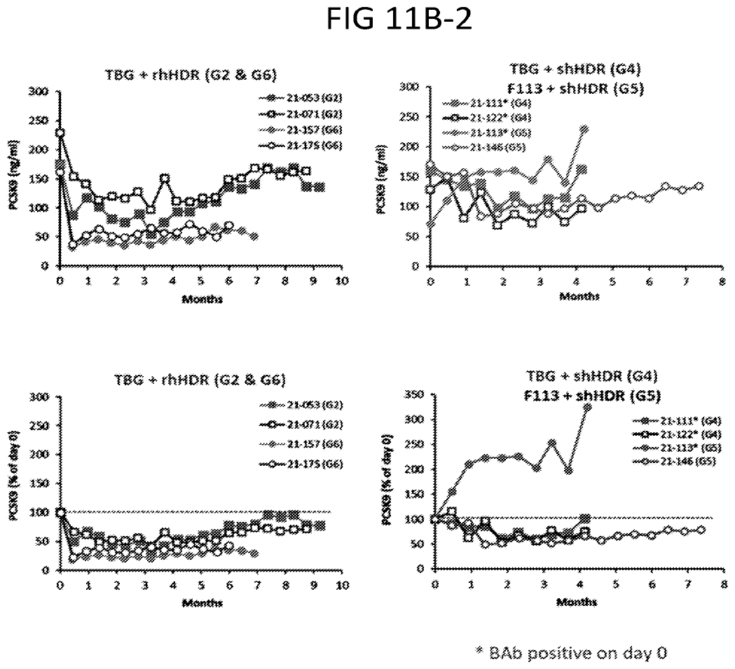

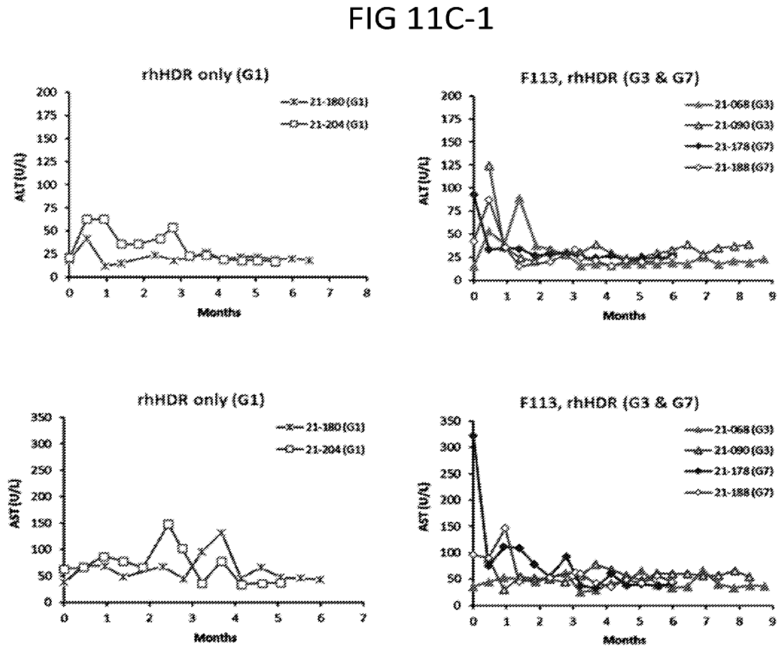

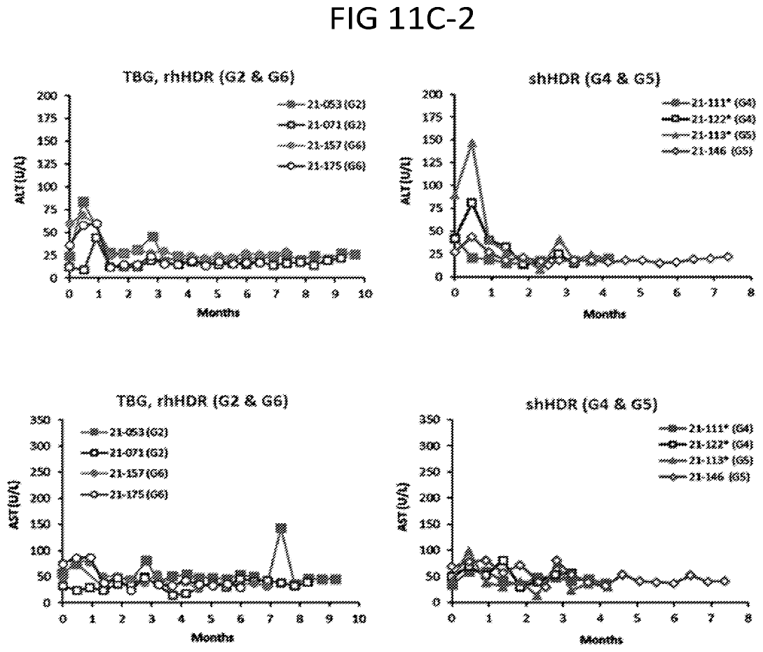

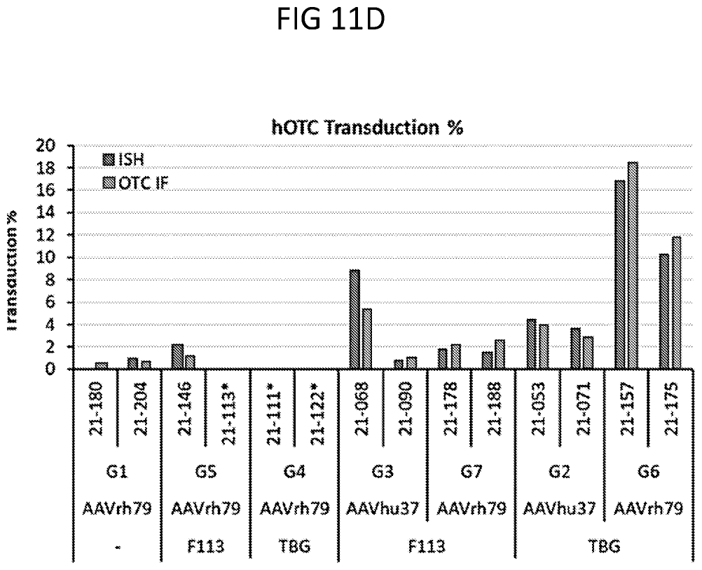

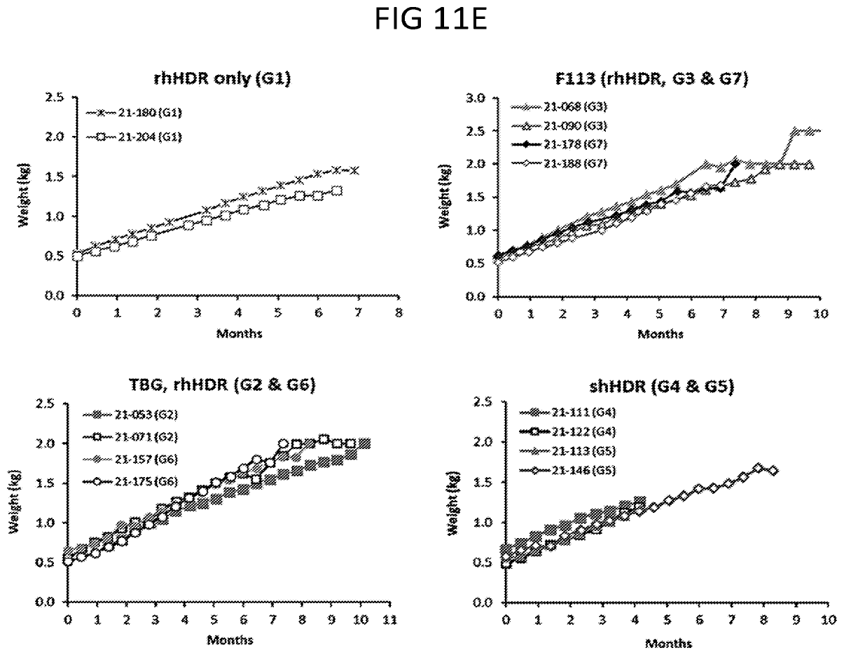

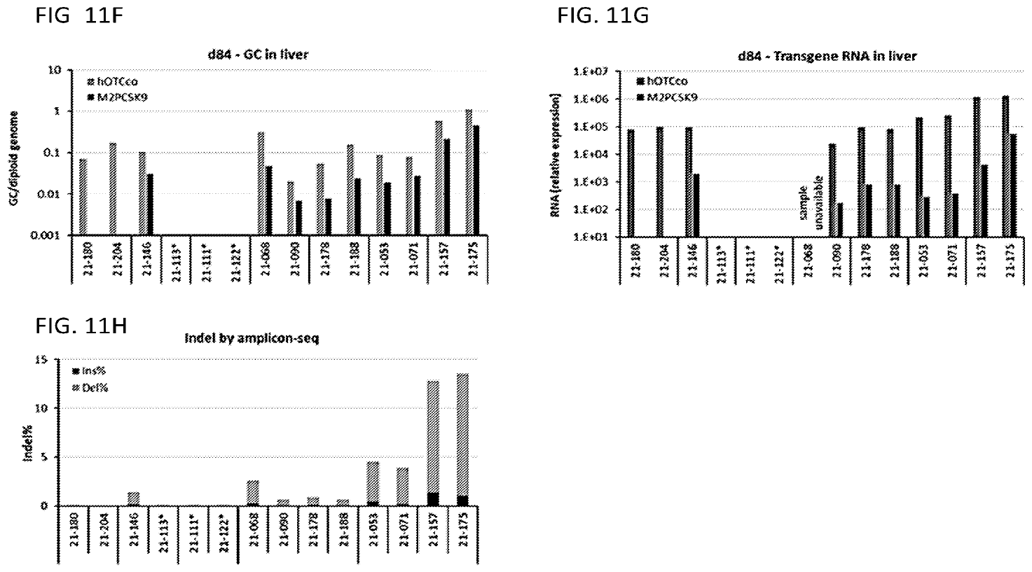

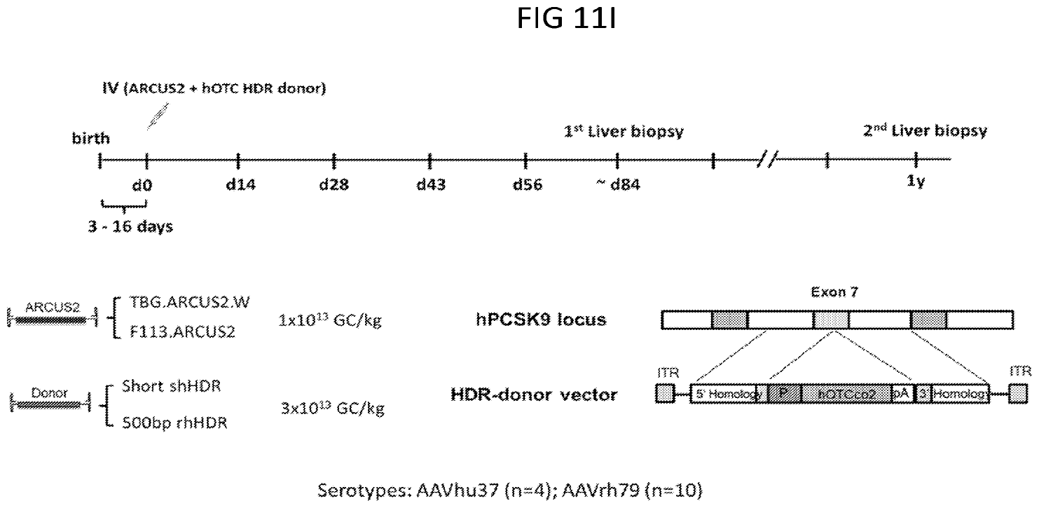

In this study newborn NHPs were administered ARCUS2 nuclease vectors, and donor vectors having HDR arms of varying length-500 bp arm or short HDR arm. Vector schematic is shown in FIG. 11I. FIG. 11A is a summary table showing data from the experiment. All 14 newborn macaques tolerated vector infusions well (i.e., no apparent clinical sequelae) and gained weight over time (FIG. 11E). Liver enzyme levels were within the normal range except for transient and modest elevation of ALT levels in some animals on Day 14 (FIG. 11C).

Analysis on the Day 0 plasma samples collected from the newborns prior to dosing showed 3 animals (21-111, 21-113, 21-122) had high levels (≥400) of binding antibodies to AAVrh79 (FIG. 11A). These pre-existing anti-AAVrh79 antibodies would block AAV gene transfer.

PCSK9 levels were followed in all newborn animals including the donor-only control animals over time. PCSK9 levels on Day 0 varied between the newborns (FIG. 11B). Nine animals showed a trend of reduced PCSK9 levels post vector administration including one donor-only control animal, while the while the remaining five animals showed persistent or transient elevation of PCSK9 levels post dosing FIG. 11B).

On Day 84, a liver biopsy via laparotomy was performed. Transduction efficiencies of hOTC in liver were evaluated by dual ISH with hOTC- and M2PCSK9-specific probes to detect transgene mRNA, and by OTC immunofluorescence to detect human OTC protein, followed by quantification on scanned slides (FIG. 11D). The three animals (21-111, 21-113, and 21-122) with pre-existing anti-AAVrh79 binding antibodies at the time of dosing did show any OTC-positive hepatocytes by both methods. The two donor-only control animals showed low level (≤1%) of hOTC transduction. The highest transduction efficiencies (11.9 and 18.6% by OTC immunofluorescence) were detected in the two animals that received a co-administration of AAVrh79.TBG.PI.M2PCSK9.WPRE.bGH and AAVrh79.rhHDR.TBG.hOTCco.bGH donor vectors (G6). Positive hOTC-expressing hepatocytes were also found to be present in clusters. These levels are above the threshold for substantially benefitting patients, which is ˜5% OTC-expressing cells.

Molecular analysis on the Day 84 liver biopsy samples from each animal were performed to measure transgene copy numbers per diploid genome, mRNA expression levels, on-target editing, and off-target editing (FIG. 11F). Consistent with the transduction efficiency analyses, the two animals (21-157 and 21-175) in Group 6 had the highest hOTC vector GC (FIG. 11F), hOTC mRNA (FIG. 11G), and on-target indel % (FIG. 11H). The M2PCSK9 vector GC in animals were 2-fold to 7-fold lower than the hOTC vector GC, while M2PCSK9 mRNA levels were 23-fold and 765-fold lower than the hOTC mRNA levels (FIG. 11F and FIG. 11G).

Off-target activity evaluated by ITR-seq identified 2 to 40 potential off-targets in the Day 84 liver biopsy samples in this study. Some off-target sites were detected in multiple animals, including the hFIX infant and hFIX newborn animals in Study 2 and Study 3, respectively. Off-target editing will be further characterized by amplicon-seq on the potential off-target sites.

In summary, we identified an M2PCSK9 vector and hOTCco donor vector combination that when co-administered into newborn macaques could achieve 12-18.6% transduction efficiency in liver at 3 months post dosing, both higher than the threshold for substantially benefitting patients, which is ˜5% OTC-expressing hepatocytes. Animals in this study are being followed for long-term efficiency and safety evaluation. We will perform a second liver biopsy at 1 year post dosing to evaluate the stability of hOTC transduction, histopathology in liver, and on- and off-targeting in liver.

### Example 4—PCSK9-HE7-KI Mouse Model

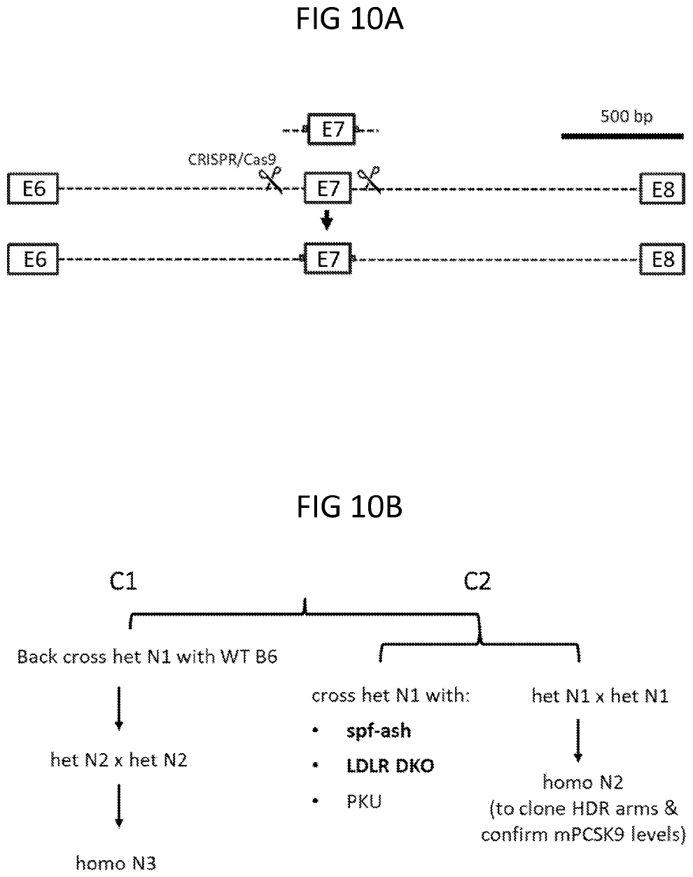

Since the M2PCSK9 targeting sequence in human and macaque PCSK9 gene is not conserved with the murine Pesk9 gene, we cannot use M2PCSK9 for genome editing in the genomic locus in mouse. Therefore, we commissioned The Jackson Laboratory to generate a knock-in mouse model that replaces a region including the exon 7 of the murine Pesk9 gene with a region of human PCSK9 gene containing exon 7, named PCSK9-hE7-KI mouse (FIG. 10A-10C). This model can be used for evaluation of in vivo genome editing and gene targeting efficiency. We then crossed PCSK9-hE7-KI mouse with sparse fur ash (spf^{ash}) mouse, spf^{ash} mice have a G to A point mutation at the splice donor site at the end of exon 4 of the Otc gene, which leads to abnormal splicing of Ote mRNA and a 20-fold reduction in both OTC mRNA and protein expression (Hodges and Rosenberg, 1989). Affected animals have 5-10% residual OTC activity and can survive on a chow diet, but they develop hyperammonia that can be lethal when on a high-protein diet (Yang et al., 2016).

The PCSK9-hE7-KI.spf^{ash} mouse model can be used for evaluation of the efficacy of in vivo gene targeting of human OTC and demonstration of correlations of targeting efficiency and efficacy. However, due to the small size of neonatal mice, evaluation of blood clinical pathology and clinical efficacy of gene targeting can only be performed after mice are weaned, once they have reached sufficient body weight, and as a terminal procedure.

FIG. 12 shows sequence alignment of 265 bp sequence represents the human PCSK9 sequence of the PCSK9-hE7 knock-in allele, mouse PCSK9 (mPCSK9) and rhesus macaques PCSK9 (rhPCSK9). There are 6 mismatches between human and rhesus sequences in this 265 bp region. Rodent and primate sequences diverge beyond this window due to insertion of various LINE and LTR. A 2 amino acid difference in exon 7 between human and mouse is present. The hE7-KI mouse is expressing normal levels of mPCSK9 by as measured ELISA.

### Example 5—In Vivo OTC Gene Targeting to the PCSK9 Locus in PCSK9-HE7-KI.Spf^{ASH} PUPS

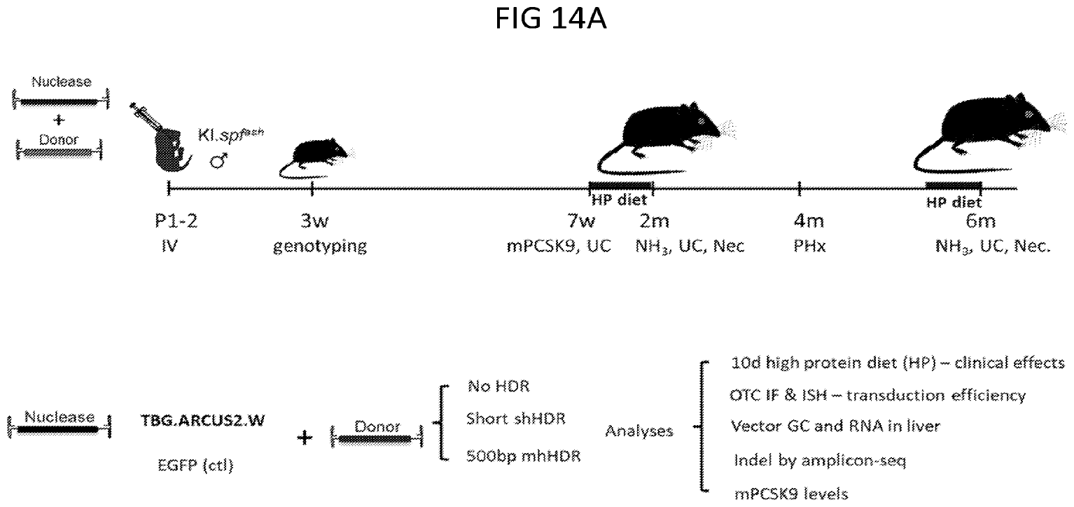

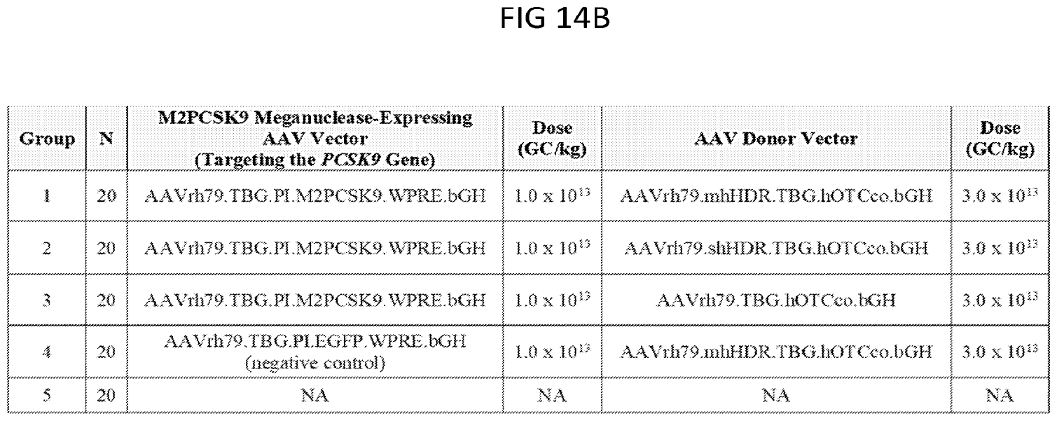

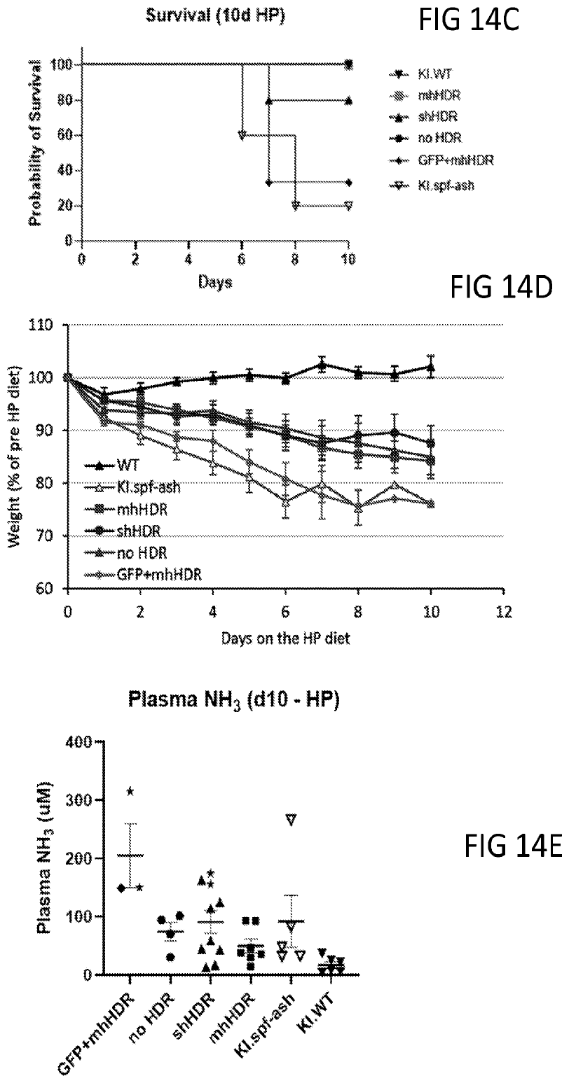

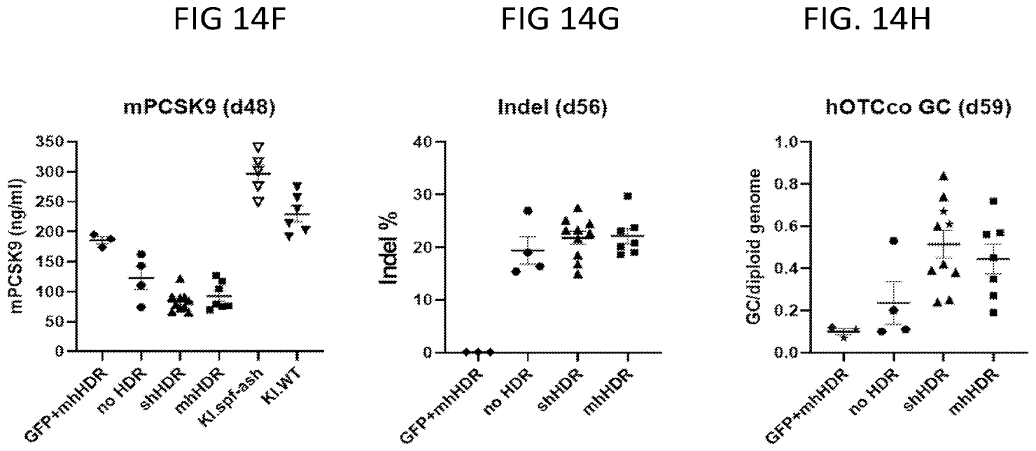

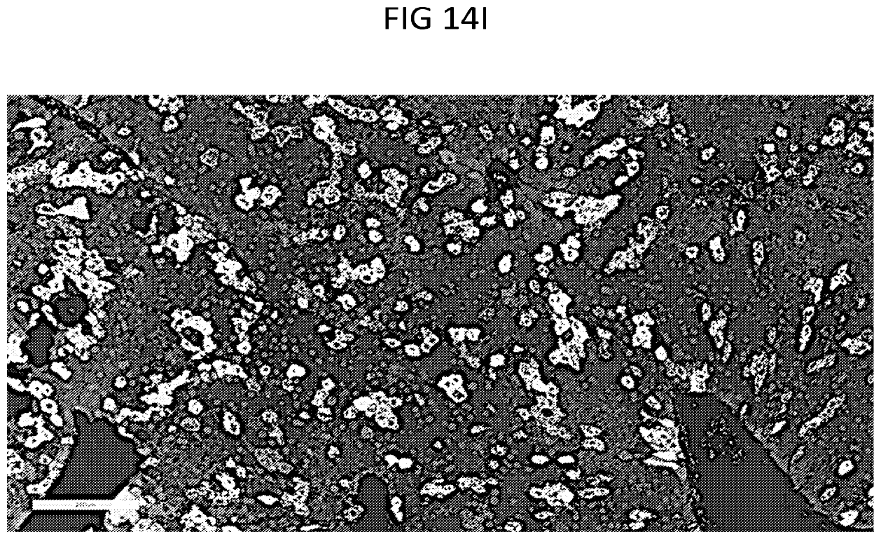

This ongoing non-GLP-compliant pharmacology study aims to assess whether M2PCSK9 meganuclease-mediated knock-in of the human OTC gene in newborn PCSK9-hE7-KI.spf^{ash} mice can achieve therapeutic human OTC expression in the target tissue for treatment of OTC deficiency (liver) following a single co-administration of an M2PCSK9 nuclease-expressing vector and a human OTC donor vector via the intended clinical ROA (IV). A schematic of the experimental design is shown in FIG. 14A, with dosage groups shown in FIG. 14B.

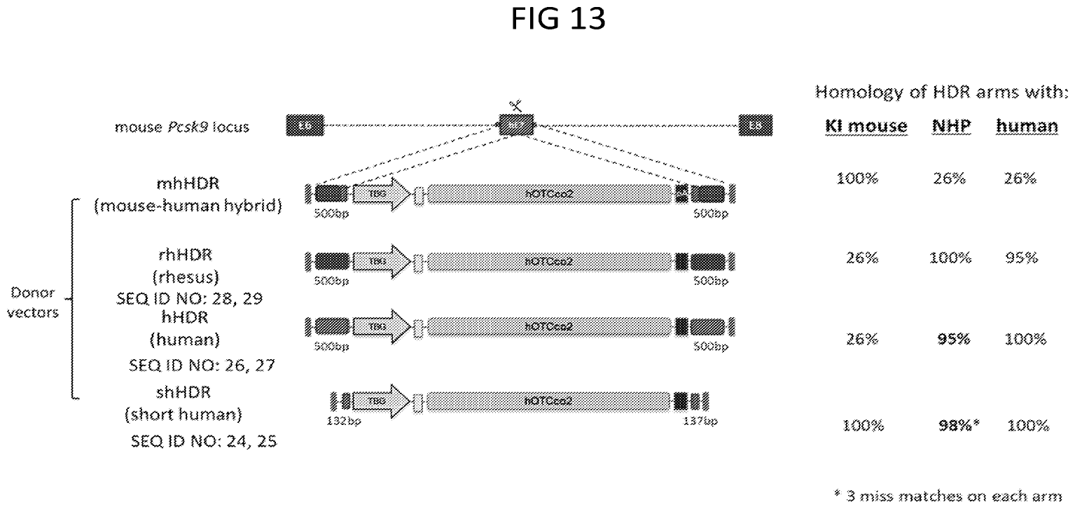

On Day 0, newborn (PND 1-2) male PCSK9-hE7-KI.spf^{ash} mice were IV co-administered an AAVrh79 vector expressing M2PCSK9 meganuclease (AAVrh79.TBG.PI.M2PCSK9.WPRE.bGH) at a dose of 1.0×10¹³ GC/kg in combination with one of three different AA Vrh79 hOTCco donor vectors at a dose of 3.0×10¹³ GC/kg. The M2PCSK9 meganuclease-expressing vector evaluated in this study (AAVrh79.TBG.PI.M2PCSK9.WPRE.bGH) was identical to the lead clinical candidate, while each hOTCco donor vector was identical to the lead clinical candidate except for the HDR arms. Specifically, while the clinical candidate includes a long version of the human HDR sequence (AAVrh79.hHDR.TBG.hOTCco.bGH), the hOTCco donor vectors assessed in this study included a mouse-human hybrid HDR sequence (AAVrh79.mhHDR.TBG.hOTCco.bGH), a shorter version of the human HDR sequence (AAVrh79.shHDR.TBG.hOTCco.bGH), or no HDR sequence (AAVrh79.TBG.hOTCco.bGH). FIG. 13 shows a comparison of the homology of the HDR arms with human, knock in mouse and NHP sequences. As a negative control, additional age-matched PCSK9-hE7-KI.spf^{ash} mice were administered an AAVrh79 vector expressing no meganuclease (AAVrh79.TBG.PI.EGFP.WPRE.bGH) in combination with AAVrh79.mhHDR.TBG.hOTCco.bGH.

In-life evaluations include viability monitoring performed daily, body weight measurements, assessment of plasma PCSK9, and plasma NH₃ and urine orotic acid levels following the high protein diet challenge, and a partial hepatectomy at Day 120 to evaluate the stability of human OTC transduction following two-third partial hepatectomy. On Days 49 and 170, a subgroup from each cohort is challenged with a 10-day high protein diet followed by necropsy at the end of the challenge. At necropsy, livers are collected to evaluate the knock-in of the human OTC gene, including assessment of human OTC mRNA expression (in situ hybridization), OTC protein expression (immunostaining), and OTC enzyme activity assessed by staining and/or an enzyme activity assay. Liver DNA is isolated to assess on-target editing (amplicon-seq. Oxford nanopore long-read sequencing) and evaluate vector genome copies.

Preliminary results show mice dosed with vectors having the mhHDR arms show survival equivalent to wild type mice, with shHDR-treated mice achieving 80% viability after the 10-day high protein diet challenge (FIG. 14C). All treated mice maintained weight better than KI-spf-ash untreated mice (FIG. 14D). Plasma ammonia levels of mHDR-treated mice were markedly reduced as compared to untreated mice (FIG. 14E).

mPCSK9 levels were measured at day 48 and all treated mice showed a reduction (FIG. 14F). Indel percentage was fairly consistent across HDR types (FIG. 14G), hOTC levels were increased in mice treated with shHDR and mhHDR (FIG. 14H). FIG. 14I shows OTC IF staining at 8 weeks.

### Example 6—In Vivo OTC Gene Targeting to the PCSK9 Locus in Newborn Rhesus Macaques

This ongoing non-GLP-compliant pharmacology study aims to assess whether M2PCSK9 meganuclease-mediated knock-in of the human OTC gene in newborn rhesus macaques can achieve therapeutic human OTC expression in the target tissue for treatment of OTC deficiency (liver) following a single co-administration of an M2PCSK9 meganuclease-expressing vector and a human OTC donor vector via the intended clinical ROA (IV).

On Day 0, newborn (1 to 16-day-old) rhesus macaques were IV co-administered one of two different vectors expressing M2PCSK9 meganuclease at a dose of 1.0×10¹³ GC/kg in combination with one of two different AAV hOTCco donor vectors at a dose of 3.0×10¹³ GC/kg. A non-nuclease group that only received the AAV hOTCco donor vector at a dose of 3.0×10¹³ GC/kg was included as donor-only controls.

For the AAV vectors targeting the PCSK9 gene, we compared two AAV vector constructs that express the M2PCSK9 in liver. AAV.TBG.PI.M2PCSK9.WPRE.bGH contains the full-length TBG promoter and two copies of enhancer elements and WPRE expresses higher levels of nuclease than AAV.TBG-S1-F113.PI.M2PCSK9.bGH, which contains a short and weak promoter. For the hOTC donor vectors, we compared two AAV.hOTCco donor vectors which differ in the length of the homology arm flanking the hOTCco transgene cassette.

NHPs were IV-administered two vectors on Day 0 and are being monitored daily for viability. In-life evaluations include measurement of body weights, clinical pathology of the blood, and gene editing analysis of plasma. Two laparotomy procedures are planned to isolate liver tissue for analysis of genome editing efficiency, vector genome copy, transgene expression, histopathology, immunostaining, and RNA ISH staining. NHPs will be followed long-term and will be necropsied (date to be determined), at which time, tissues from the liver and other major organs will be collected for evaluation of genome editing efficiency, vector genome copy, transgene expression, histopathology, immunostaining, and RNA ISH staining.

### Example 7—Assessing the Efficacy and Determining the Ratio of Vectors in PCSK9-HE7-KI.SPF^{ASH} Pups

This non-GLP-compliant pharmacology study assesses the ratio of vector components required to achieve the highest efficacy of M2PCSK9 meganuclease-mediated knock-in of the human OTC gene in newborn PCSK9-hE7-KI.spf^{ash} mice for treatment of OTC deficiency (liver) following a single co-administration of an M2PCSK9 nuclease-expressing vector and a human OTC donor vector via the intended clinical ROA (IV).

On Day 0, newborn (PND 1-2) male PCSK9-hE7-KI.spf^{ash} mice will be IV co-administered an AAVrh79 vector expressing M2PCSK9 meganuclease (AAVrh79.TBG.PI.M2PCSK9.WPRE.bGH) at one of three doses in combination with one of three doses of the hOTCco donor vector including a mouse-human hybrid HDR sequence (AAVrh79.mhHDR.TBG.hOTCco.bGH). The M2PCSK9 meganuclease-expressing vector evaluated in this study (AAVrh79.TBG.PI.M2PCSK9.WPRE.bH) is identical to the clinical candidate, while the hOTCco donor vector is identical to the clinical candidate except for the HDR arms. Specifically, while the clinical candidate includes a long version of the human HDR sequence (AAVrh79.hHDR.TBG.hOTCco.bGH), the hOTCco donor vectors assessed in this study included a mouse-human hybrid HDR sequence (AAVrh79.mhHDR.TBG.hOTCco.bGH).

The mouse-human hybrid HDR sequence within the donor vector (AAVrh79.mhHDR.TBG.hOTCco.bGH) was selected for this study to enable evaluation of the pharmacology of this approach where the donor sequence is directly homologous to the sequence in the PCSK9-hE7-KI.spf^{ash} mice.

In-life evaluations include viability monitoring performed daily, body weight measurements, assessment of plasma NH₃ and urine orotic acid levels following the high protein diet challenge. On Day 81, mice will be challenged with a 10-day high protein diet followed by necropsy at the end of the challenge. At necropsy, livers will be collected to evaluate the knock-in of the human OTC gene, including assessment of human OTC mRNA expression (in situ hybridization), OTC protein expression (immunostaining), and OTC enzyme activity assessed by staining and/or an enzyme activity assay. Liver DNA will also be isolated to assess on-target editing (amplicon-seq) and evaluate vector genome copies.

### Example 8—Assessing the Efficacy and Determining the Minimum Effective Dose in PCSK9-HE7-KI.SPF^{ASH} Pups

This GLP-compliant pharmacology evaluates the efficacy and determines the MED of IV-administered AAV in the newborn PCSK9-hE7-KI.spf^{ash} mouse model. The AAVrh79 vector expressing M2PCSK9 meganuclease (AA Vrh79.TBG.PI.M2PCSK9.WPRE.bGH) is the toxicological vector lot that will be manufactured for the planned GLP-compliant toxicology study. Instead of utilizing the test article, which includes a long version of the human HDR sequence (AAVrh79.hHDR.TBG.hOTCco.bGH), this study will utilize the hOTCco donor vector that includes the mouse-human hybrid HDR sequence (AAVrh79.mhHDR.TBG.hOTCco.bGH). This vector will be manufactured in a comparable method to that of the toxicological vector lot of the clinical candidate.

We have chosen to use the mouse-human hybrid HDR sequence within the donor vector (AAVrh79.mhHDR.TBG.hOTCco.bGH) for this study to enable us to effectively study the pharmacology of this approach where the donor sequence is directly homologous to the sequence in the PCSK9-hE7-KI.spf^{ash} mice.

This study will evaluate N=60 neonatal (PND 1-2) newborn PCSK9-hE7-KI.spf^{ash} mice and N=15 age-matched male PCSK9-hE7-KI.WT (wild type) as controls. The study will include one necropsy time point (90 days). For efficacy evaluation, mice will be challenged by a 10-day course of a high protein diet from Day 81 to Day 90. Survival, body conditions, and biomarker changes will be evaluated. Three dose levels of AAV will be evaluated using IV administration. The dose levels will be selected based on the range of doses evaluated in previous nonclinical studies. The dose levels evaluated will bracket the anticipated clinical doses.

In-life assessments will include daily viability checks, monitoring for survival, body weight measurements, assessment of serum PCSK9 levels, plasma NH₃ and urine orotic acid levels following high protein diet challenge. Necropsies will be performed on Day 90. At necropsy, blood will be collected for CBC/differentials and serum clinical chemistry analysis. A list of tissues will be collected for histopathological evaluation. Liver will be collected to evaluate the knock-in of the human OTC gene, including assessment of human OTC mRNA expression (in situ hybridization), OTC protein expression (immunostaining), and OTC enzyme activity assessed by staining and/or an enzyme activity assay. Liver DNA will also be isolated to assess on-target editing (amplicon-seq) and evaluate vector genome copies.

The MED will be determined based upon analysis of survival following the high protein diet, plasma NH₃ levels at the end of the high protein diet challenge human OTC mRNA and protein expression, OTC enzyme activity, and on-target editing of AAV-treated newborn PCSK9-hE7-KI.spf^{ash} compared to vehicle-treated newborn PCSK9-hE7-KI.spf^{ash} control mice.

### Example 9—Toxicology Study IN PCSK9-HE7-KI.SPF^{ASH} Pups

A 6-month GLP-compliant safety study will be conducted in newborn (PND 1-2) PCSK9-hE7-KI.spf^{ash} mice to investigate the safety, tolerability, pharmacology, and pharmacokinetics of the test article following IV co-administration. Interim analyses, including on-target editing, off-target editing, transgene expression, and histopathological analyses, will be performed on Day 60 and Day 180 as these time points will allow sufficient time for the nuclease-dependent gene insertion to have reached stable plateau levels following administration. Newborn PCSK9-hE7-KI.spf^{ash} mice will receive one of three dose levels of the test article (1.0×10¹² GC/kg nuclease vector and 3.0×10¹² GC/kg donor vector, 3.3×10¹² GC/kg nuclease vector and 1.0×10¹³ GC/kg donor vector, or 1.0×10¹³ GC/kg nuclease vector and 3.0×10¹³ GC/kg: N=20 per dose) or vehicle (phosphate-buffered saline [PBS]; N=20). After the test article or vehicle administration, in-life evaluations will include clinical observations to monitor daily for signs of distress and abnormal behavior, body weight measurements, and blood clinical serum chemistry (specifically ALT, AST, and total bilirubin).

On Day 60 after test article administration, cohorts 1, 3, 5, and 7 of will be euthanized, and a histopathological analysis will be performed on a comprehensive list of tissues, including, but not limited to, brain, spinal cord, heart, liver, spleen, kidney, lungs, reproductive organs, adrenal glands, and lymph nodes. Organs will be weighed as appropriate.

Liver samples will be collected and analyzed for on-target editing by amplicon-seq and AMP-seq, off-target editing by ITR-seq and amplicon-seq, vector biodistribution, and transgene expression. In liver samples, biodistribution will be evaluated by PCR and meganuclease RNA expression will be analyzed by RT-PCR will be performed. Highly perfused organs will be analyzed for meganuclease RNA and tissues with detectable expression of meganuclease RNA will be evaluated for on-target editing by amplicon-seq.

Tissues with detectable on-target editing will be further evaluated for off-target editing. For vector biodistribution, qPCR detection specific to the transgenes of the dual components, M2PCSK9 and hOTCco, will be developed. The efficiency, linearity, precision, reproducibility and limit of detection of the assays will be assessed using the AAV cis plasmids as surrogates of target sequence. The lower limit of quantification (LLOQ) of the assays will be determined prior to the assay on test tissues or excreta initiated. A qualification plan will be implemented to bridge the transgene specific assays to the qualification studies conducted previously. The matrix tested will include intended target, liver for biodistribution. The matrix effect will be further evaluated based on the recovery of spiked target controls from all samples tested in the course of biodistribution studies as well as the data subtracted from the qualification studies conducted previously.

### Example 10—HLDLR Mini Gene Knock-In in PCSK9 Locus by SACAS9 in PCSK9-HE7-KI.LDLR⁻/LDLR⁻. APOBEC⁻/APOBEC⁻ Pups (HOFH Model)

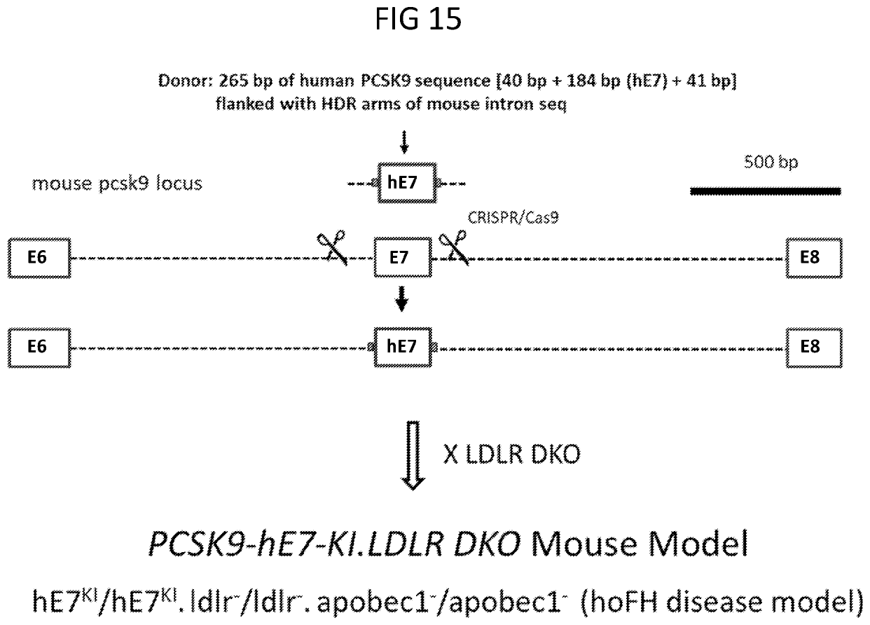

This study aims to assess whether Cas9-mediated knock-in of the human LDLR gene in newborn PCSK9-hE7-KI.ldlr⁻/ldlr⁻.apobec⁻/apobec mice can achieve therapeutic human LDLR expression in the target tissue for treatment of familial hypercholesterolemia (liver) following a single co-administration of a SaCas9 nuclease-expressing vector and a human LDLR donor vector via the intended clinical ROA (IV). A mouse model was generated using the experimental design in FIG. 15. In the mouse model, mouse PCSK9 exon 7 is replaced with human PCSK9 exon 7, that contains the SaCas9 targeting sequences.

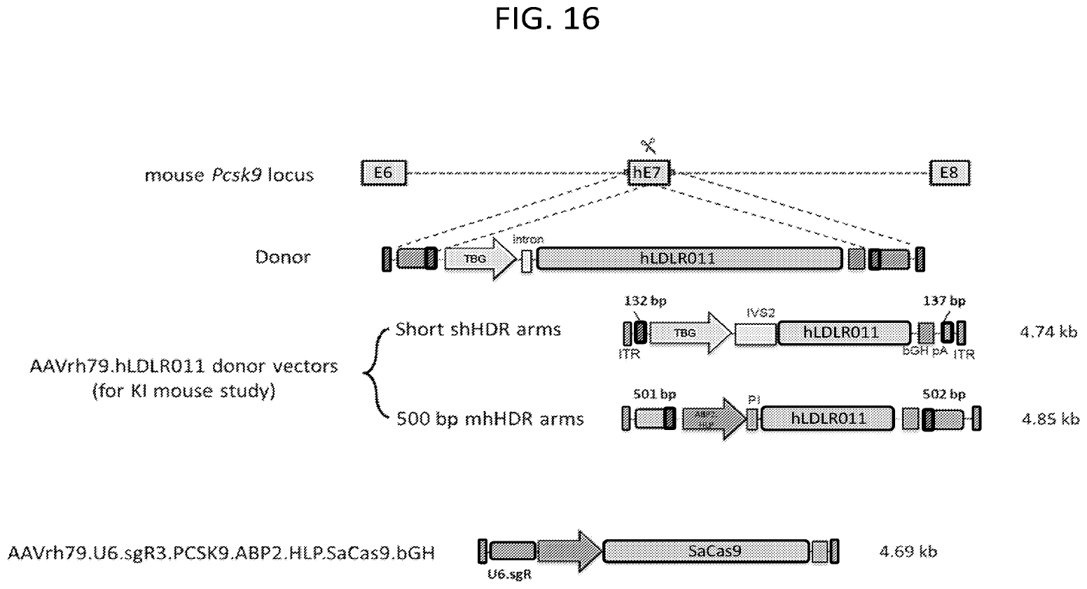

On Day 0, newborn PCSK9-hE7-KI.ldlr⁻/ldlr⁻.apobec⁻/apobec mice were IV co-administered an AAVrh79 vector expressing Cas9 (AAVrh79.U6.sgR3.PSCK9.APB2.HLP.SaCas9.bGH) at a dose of 1.0×10¹³ GC/kg in combination with one of two different AAVrh79 hLDLR donor vectors at a dose of 3.0×1013 GC/kg. A schematic showing the vectors used is shown in FIG. 16. Specifically, one of the donor vectors assessed in this study included a mouse-human hybrid HDR sequence (AAVrh79.mhHDR.hLDLR011) and the other included a shorter version of the human HDR sequence (AA Vrh79.shHDR.hLDLR011). As a negative control, additional age-matched PCSK9-hE7-KI.spf^{ash} mice were administered an AAVrh79 vector expressing no saCas9 in combination with AAVrh79.shHDR.hLDLR011.

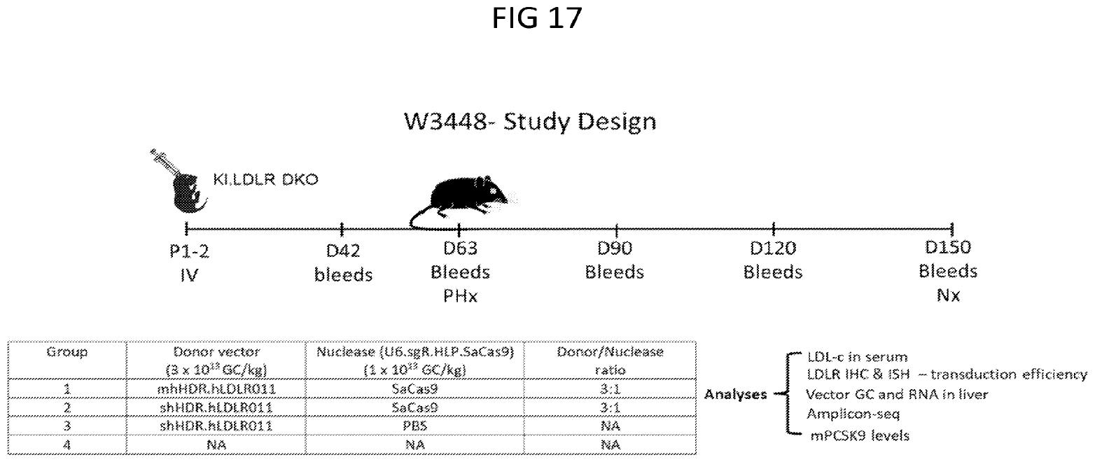

In-life evaluations include viability monitoring performed daily, and assessment of serum LDL-c levels at days 42, 63, 90, 120, and 150. A partial hepatectomy at Day 63 to evaluate the stability of human LDLR transduction, and necropsy at date 150. At necropsy, livers are collected to evaluate the knock-in of the human LDLR gene, including assessment of human LDLR mRNA expression (in situ hybridization), LDLR protein expression (immunostaining). Liver DNA is isolated to assess on-target editing (amplicon-seq, Oxford nanopore long-read sequencing) and evaluate vector genome copies. Experimental design is shown in FIG. 17.

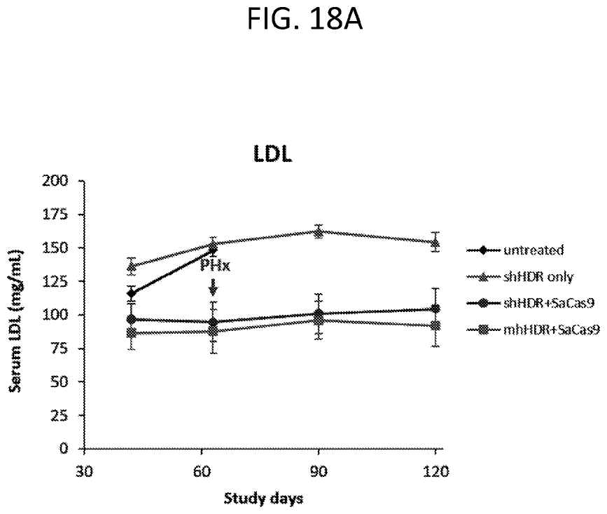

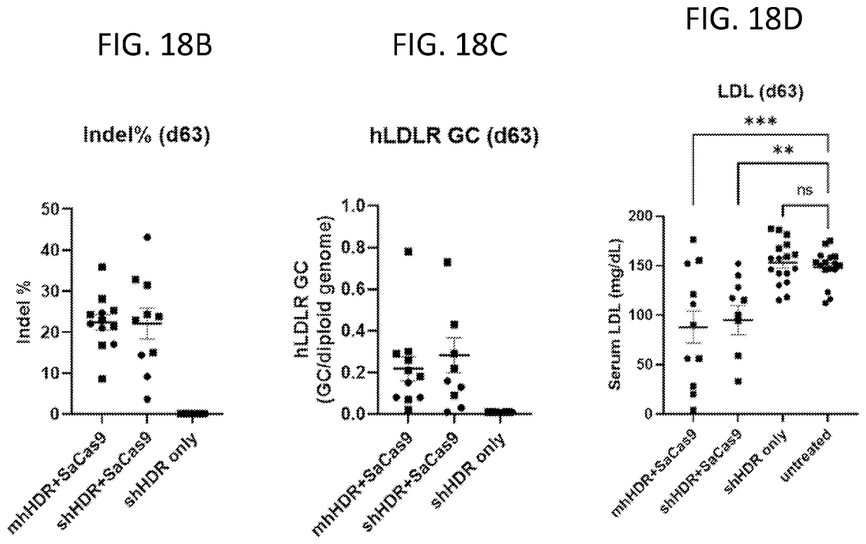

Preliminary results show mice dosed with saCas9 and donor vectors had significantly reduced serum LDL levels. There was no change of LDL following 2/3 partial hepatectomy indicating stable integration (FIG. 18A). Indels were consistent using mhHDR and shHDR donor vectors (FIG. 18B). At day 63, shHDR-treated mice showed slightly higher hLDLR levels (FIG. 18C), while serum LDL levels were similar for mhHDR and shHDR (with saCas9) vectors (FIG. 18D).

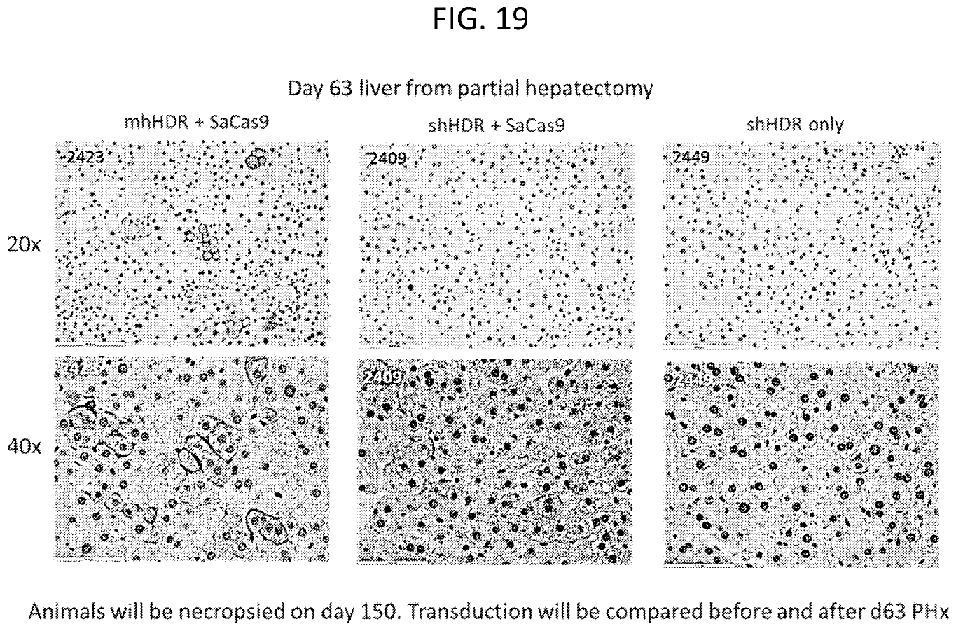

FIG. 19 shows immunohistochemistry evaluation of hLDLR expression in day 63 liver following partial hepatectomy.

### Example 11—hFIX Mini Gene Knock-In in Pcsk9 Locus by Arcus in Adolescent Rhesus Macaques

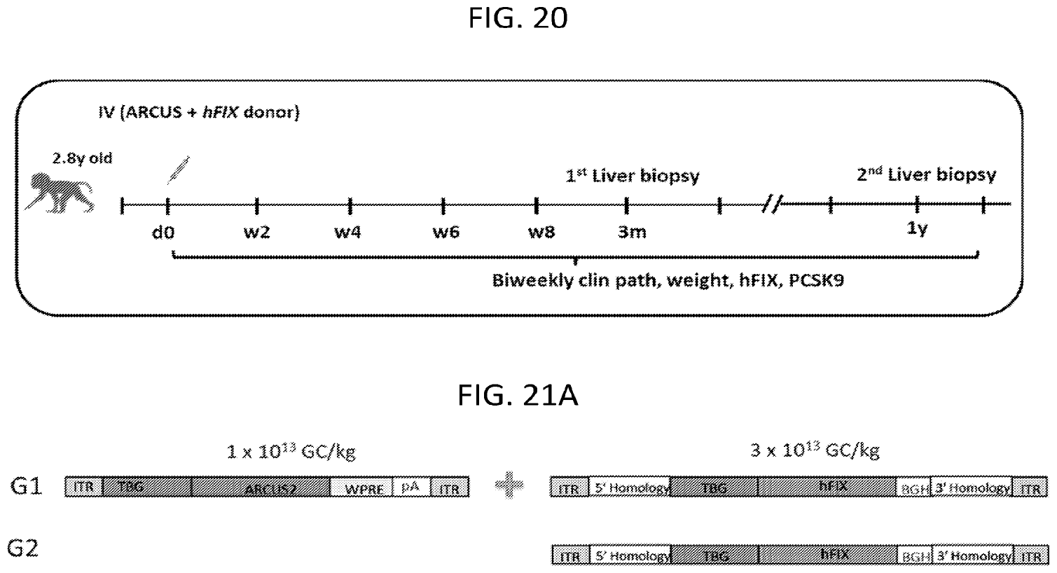

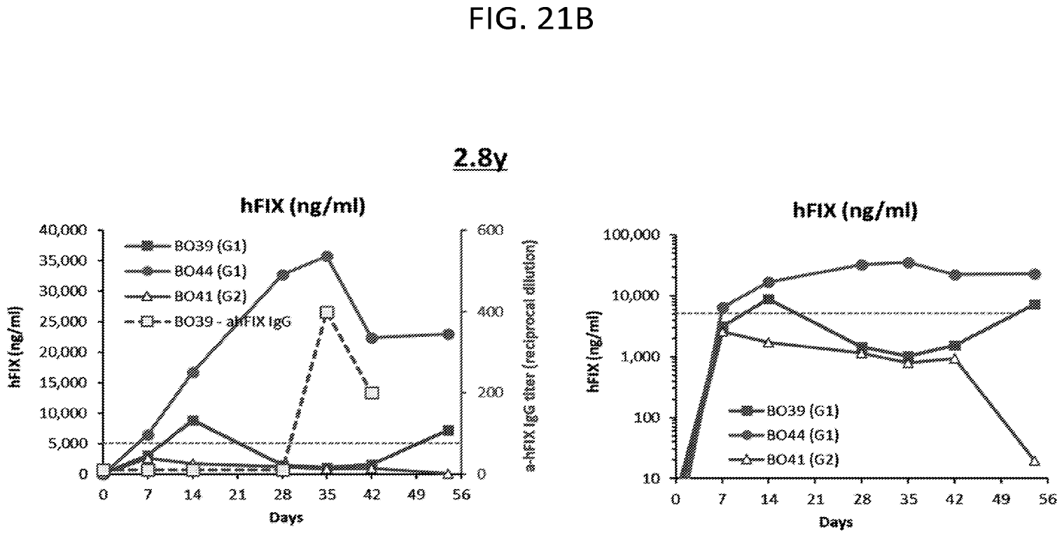

A study was performed to assess the effects of ARCUS-mediated knock-in of the human FIX gene in adolescent macaques (2.8 years of age) as compared to AAV-delivery of the transgene only. An overview of the experimental design is shown in FIG. 20. Schematics of the vector genomes delivered are shown in FIG. 21A.

On Day 0, adolescent rhesus macaques were IV co-administered an AAVhu37 vector expressing ARCUS (TBG.PI.PCS 7-8L. 197.WPRE.bGH.KanR: SEQ ID NO: 85) at a dose of 1.0×10¹³ GC/kg in combination with a AA Vhu37 hFIX donor vector (rhHDR-PCSK9-ARCUS.TBG.IVS2.hFIXco3T.BC06.bGH: SEQ ID NO: 83) at a dose of 3.0×10¹³ GC/kg in Group 1 (G1). In Group 2 (G2), animals were dosed with AAVhu37 hFIX donor vector only at a dose of 3.0×10¹³ GC/kg.

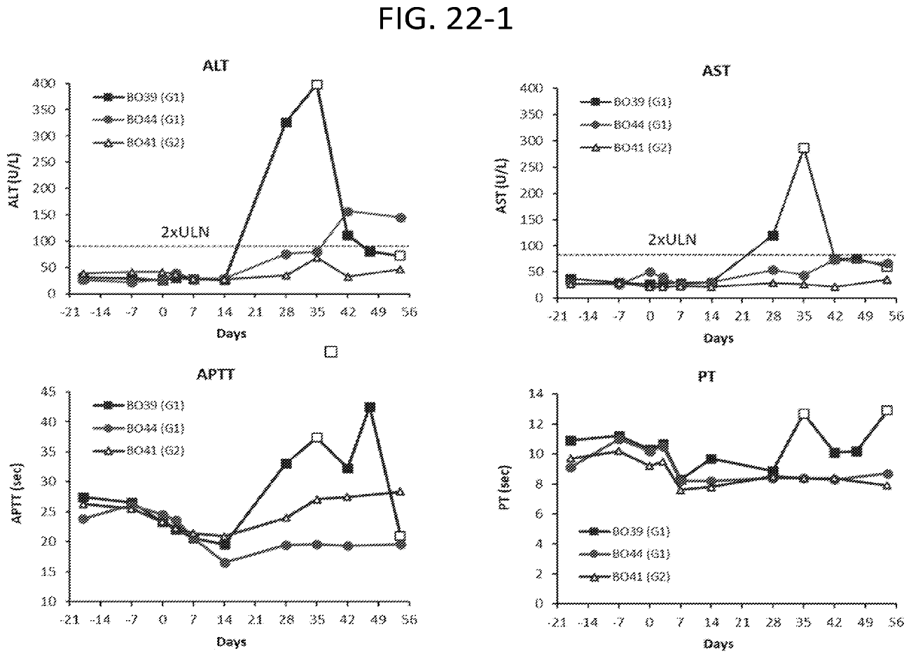

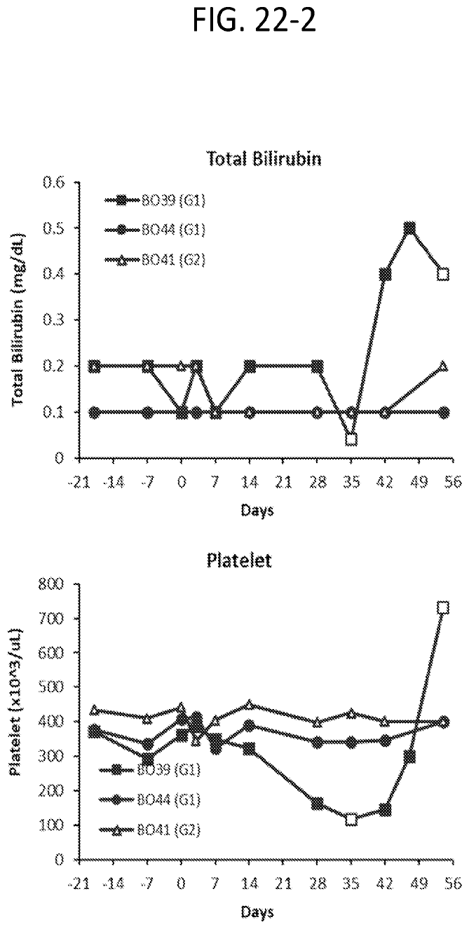

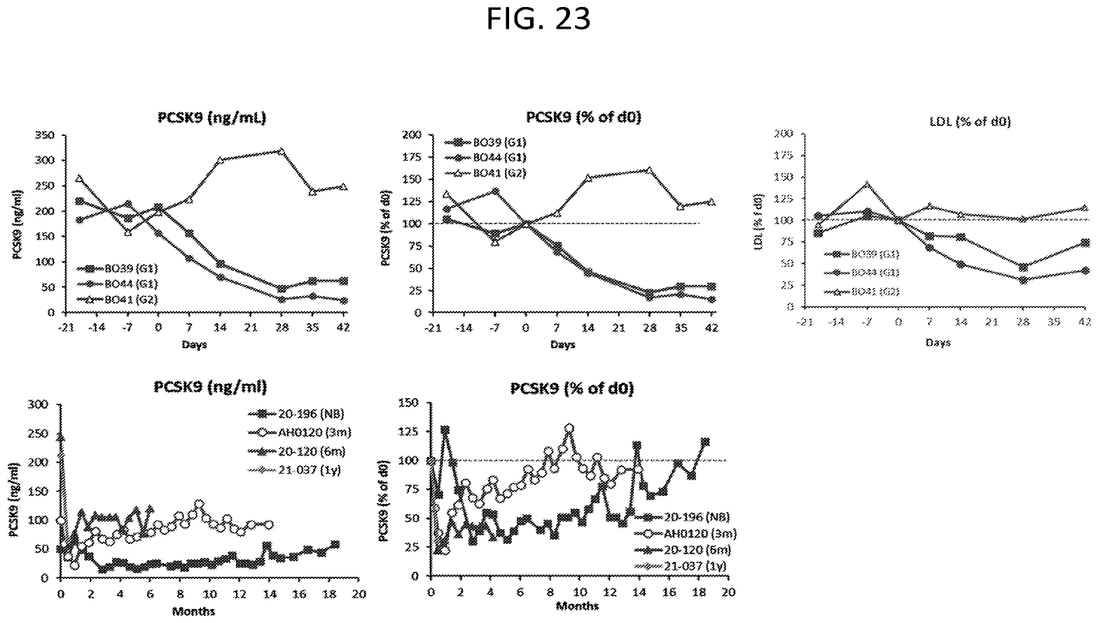

Preliminary results showed animals dosed with ARCUS and donor vectors had significantly increased hFIX levels, as compared to delivery of the AAV vector alone. The dashed line in FIG. 21B indicates normal levels. Liver enzymes were elevated in the BO39 animal (FIG. 22), and oral steroids were administered. PCSK9 levels were significantly reduced in G1 animals but not in G2 animals (FIG. 23).

### Example 12—hFIX Mini Gene Knock-In in Pcsk9 Locus by Arcus in Rhesus Macaques of Different Ages

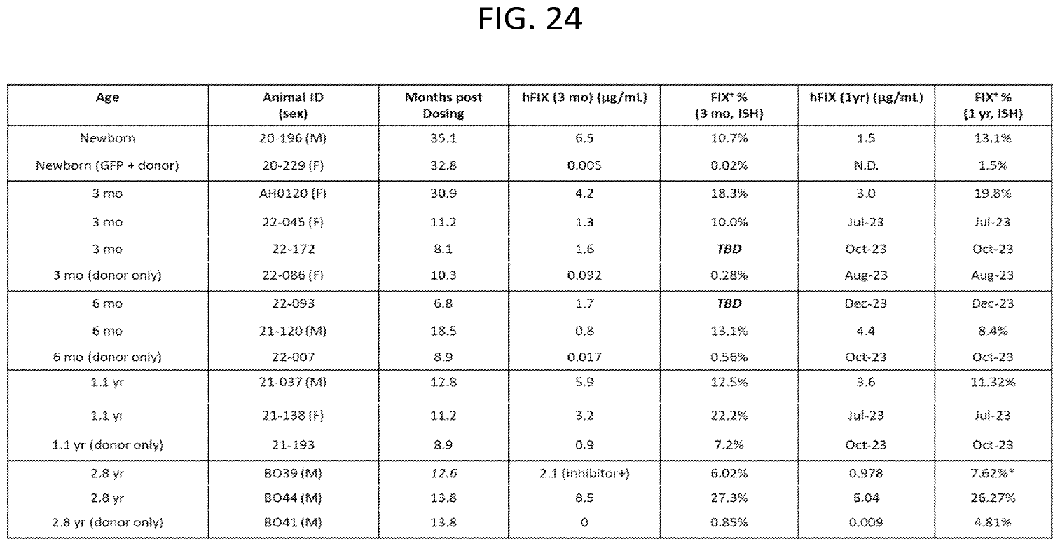

A study was performed to assess the effects of ARCUS-mediated knock-in of the human FIX gene in macaques of different ages. The animals described in Example 11 were included as the 2.8 year age group. An overview of the animals evaluated and results is shown in FIG. 24.

On Day 0, rhesus macaques were co-administered (IV) an AAVhu37 vector expressing ARCUS (TBG.PI.PCS 7-8L. 197.WPRE.bGH.KanR: SEQ ID NO: 85) at a dose of 1.0×10¹³ GC/kg in combination with an AAVhu37 hFIX donor vector (rhHDR-PCSK9-ARCUS.TBG.IVS2.hFIXco3T.BC06.bGH: SEQ ID NO: 83) at a dose of 3.0×10¹³ GC/kg, or dosed with AAVhu37 hFIX donor vector only at a dose of 3.0×10¹³ GC/kg.

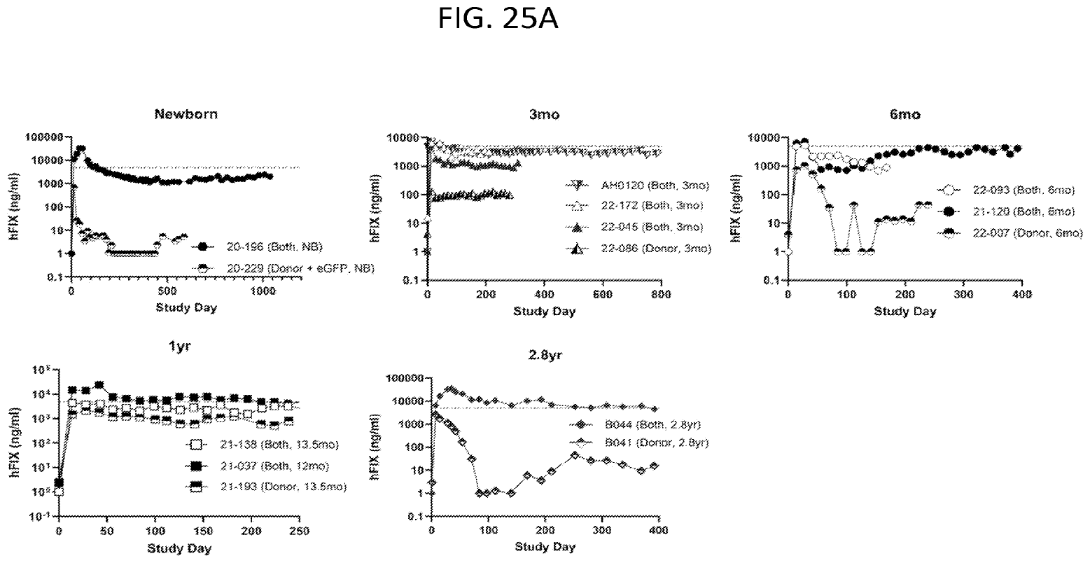

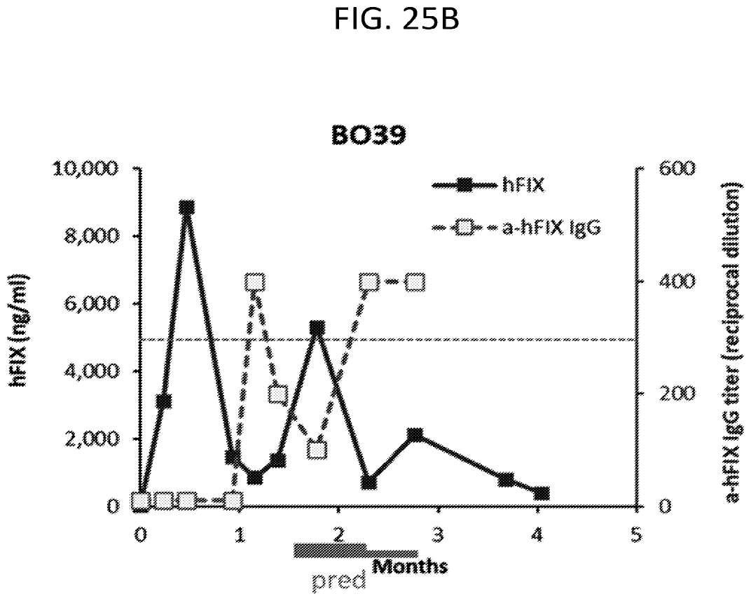

FIG. 25A and FIG. 25B show hFIX expression levels in the animals of different ages, hFIX levels were above normal in both the 1.1 year and 2.8 year cohorts. Animal BO39 showed an anti-hFIX IgG response and was treated with prednisolone from days 48-84.

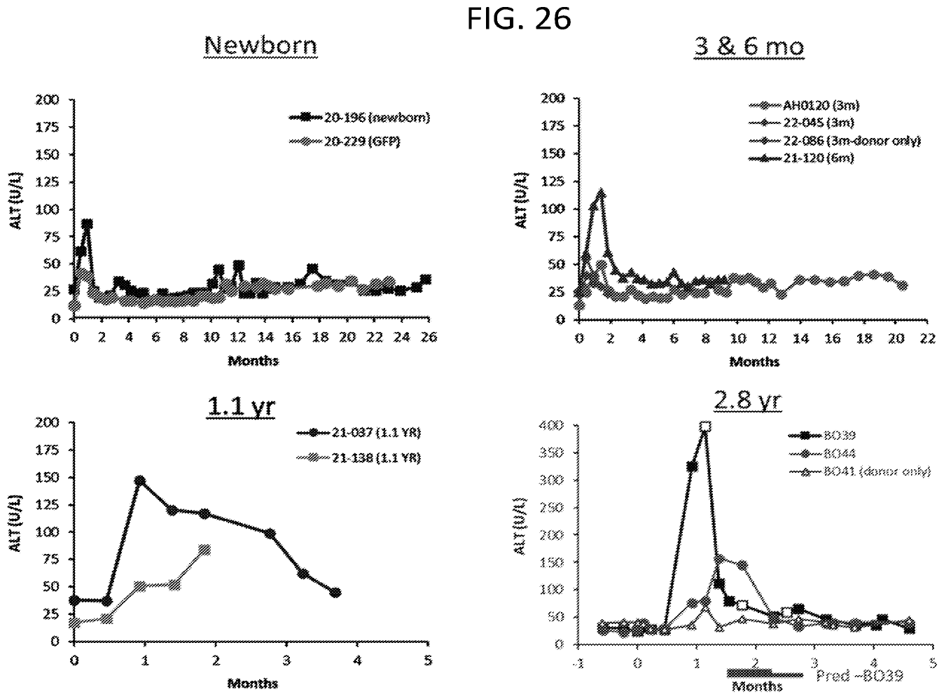

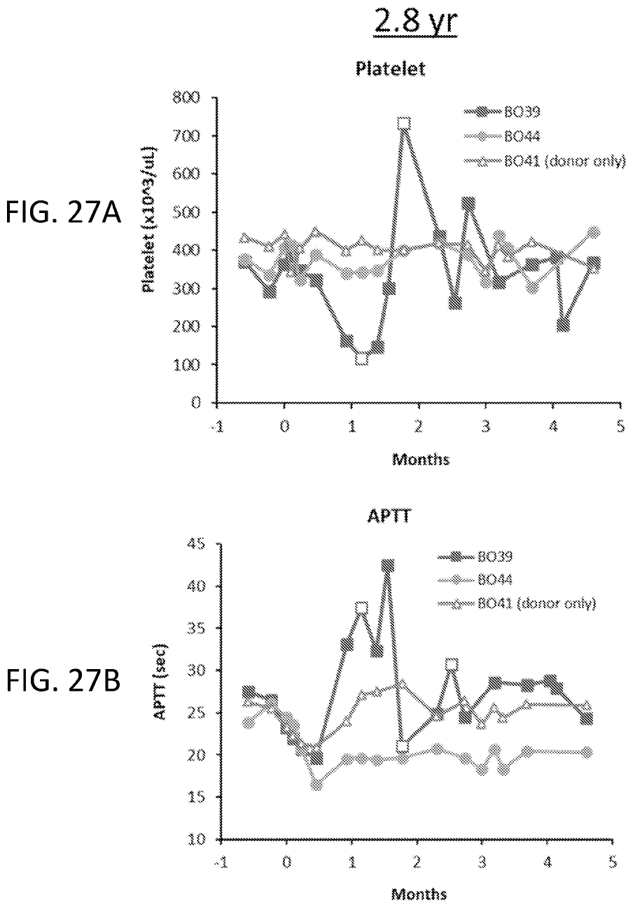

FIG. 26 shows ALT levels in the animals of the noted ages. FIG. 27A shows platelet levels, and FIG. 27B shows APTT levels for BO39, BO44, and BO41. Animal BO39 showed an ALT, platelet, and APTT response 1-2 months after dosing.

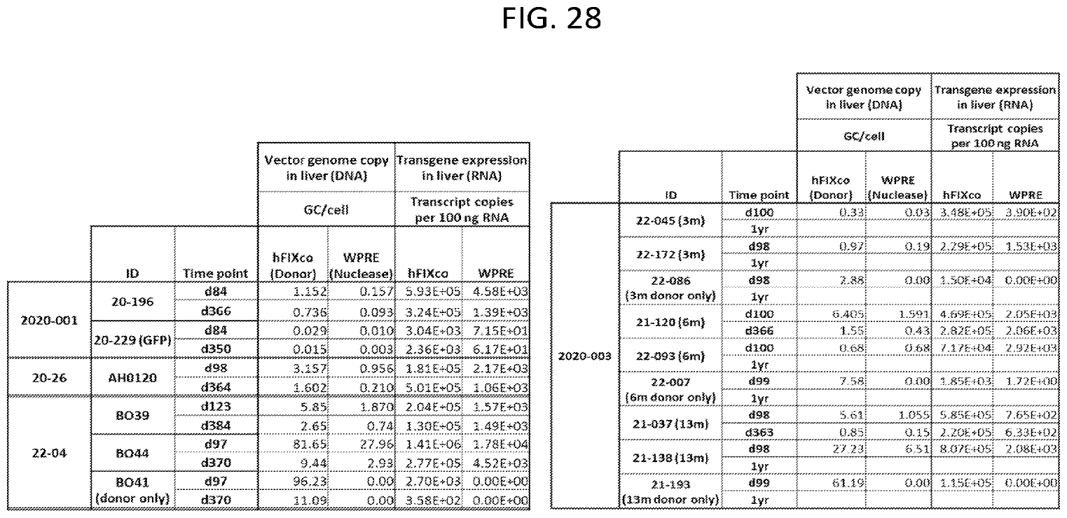

FIG. 28 shows results from quantification of RNA and DNA for detection of transgene (hFIX) and nuclease (ARCUS) in liver.

### Example 13—Analysis of the Optimal Ratio for Nuclease and Donor AAV Vectors in Ornithine Transcarbamylase Deficiency Treatment

Following the highly successful studies in newborn rhesus macaques above, this study aimed to evaluate the impact of vector ratio for the ARCUS meganuclease (AAVrh79.TBG.PI.M2PCSK9.WPRE.bGH) and donor vectors (AAVrh79.mhHDR.TBG.hOTCco.bGH).

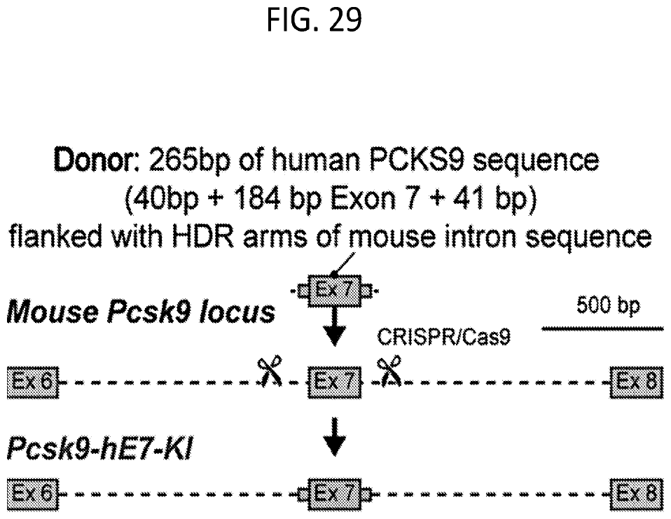

These studies were performed in a mouse OTCD model (FIG. 29) rendered susceptible to the M2PCSK9 ARCUS meganuclease via germ-line modification of exon 7 of the endogenous Psck9. After injecting the two vectors into newborn mice at a ratio of 1:1 or 1:3 for the ARCUS meganuclease to mouse OTC donor vectors, the mice were challenged with a high-protein diet (HPD) to induce lethal hyperammonemia. The mice were then evaluated for survival and body weight changes.

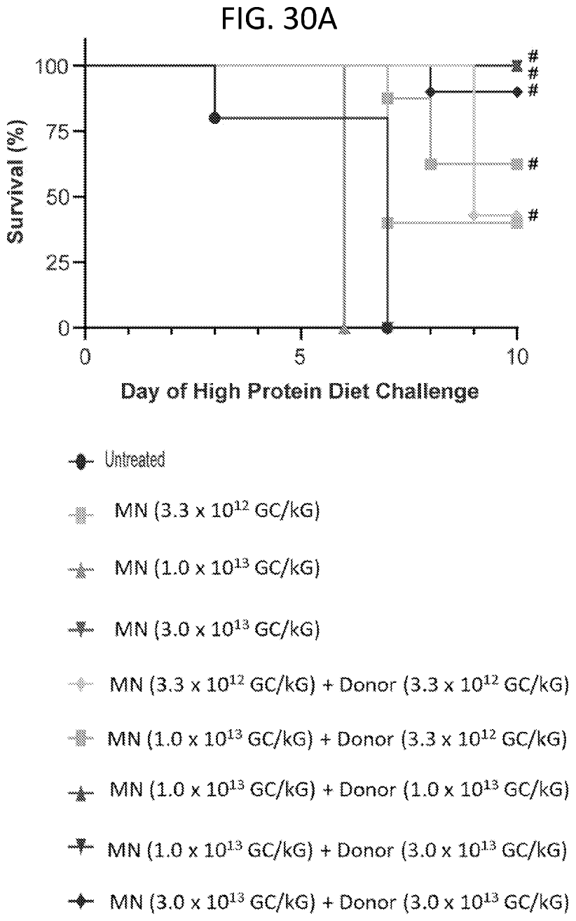

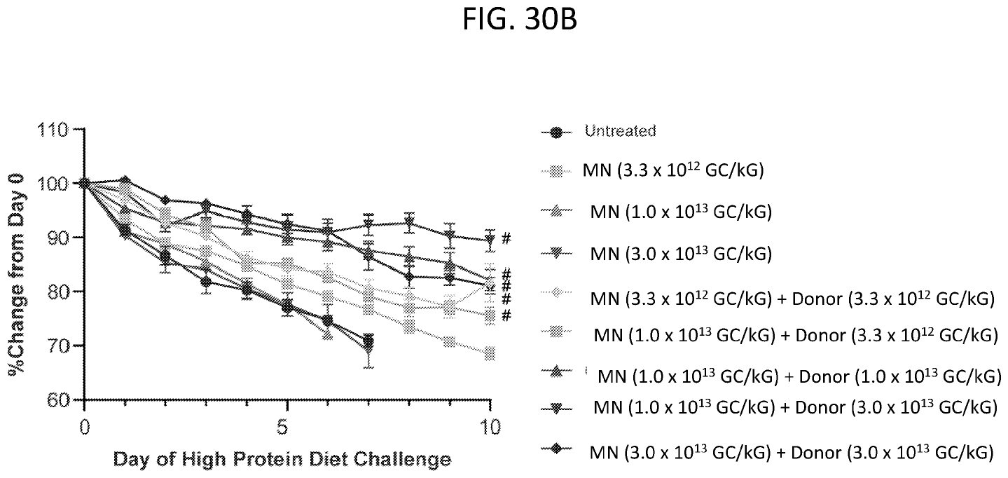

Newborn KI.spf^{ash} (OTCD) mice (PND 0-1) were injected IV with a single dose of test article (the meganuclease either alone or in combination with the donor vector) or were untreated. HPD challenge was initiated on Day 50±2 and continued for 10 days. Mice were monitored for survival throughout. During the HPD, mice were euthanized for clinical signs and/or >20% body weight loss. All mice treated with the combination of the meganuclease with the donor vector showed significant improvement in survival when compared to the untreated control group. (FIG. 30A) Body weights for each mouse were also measured daily throughout the HPD challenge. (FIG. 30B) All mice treated with the combination of the meganuclease with the donor vector showed significantly improved body weight when compared to the untreated control group.

All untreated OTCD mice had to be euthanized due to body weight loss >20% (n=5). Survival was significantly increased for all groups administered the meganuclease in combination with the donor vector. Mice injected with a low vector dose at a ratio of either 1:1 or 1:3 exhibited reduced weight loss at day 6 of the HPD challenge (89%-91% of Day 0). Increasing the 1:1 ratio dose maintained body weights at 91% of Day 0 values (no significant difference).

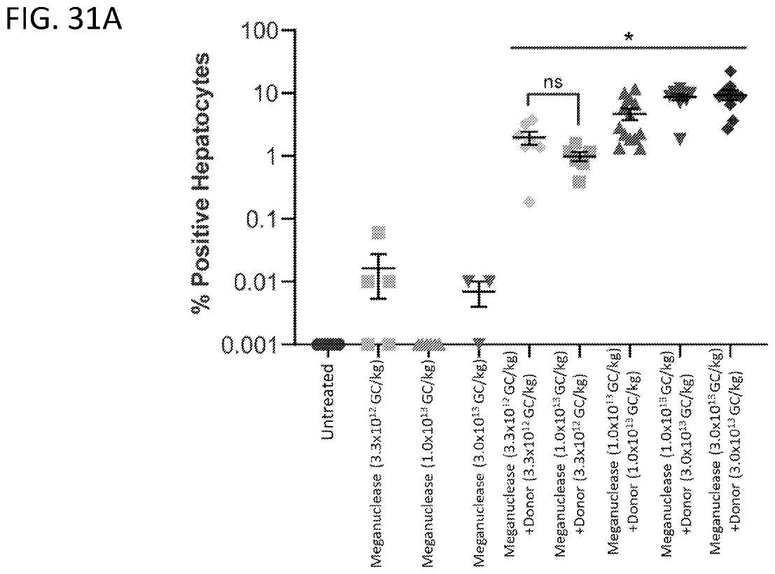

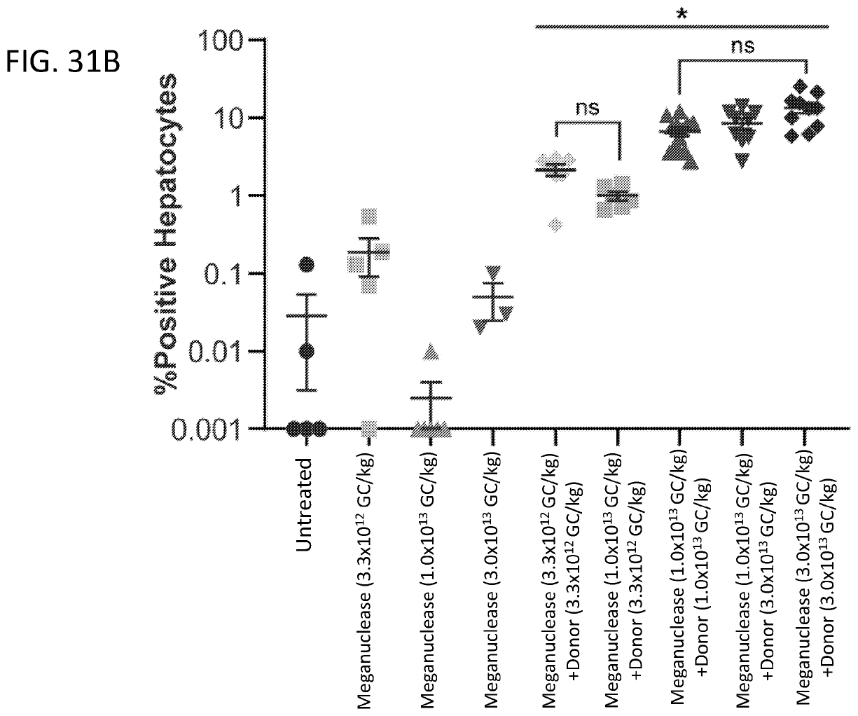

Next, on Day 60±2, mice that completed the 10-day HPD challenge were euthanized and necropsied. The liver was harvested from necropsied mice, formalin fixed, paraffin embedded, and hOTC ISH and IF were performed. The percentages of hepatocytes that were positive for the ISH (FIG. 31A) or IF signal (FIG. 31B) were quantified. All mice treated with the combination of the meganuclease with the donor vector showed significant higher percent of hepatocytes that were positive for the ISH and IF signals when compared to the untreated control group.

There was not a significant difference in RNA transcript or protein levels with increasing dose of the donor vector at the 1:1 ratio, but there were significant differences between groups administered the 1:1 or 1:3 ratio, with the highest RNA expression detected at the 1:3 ratio.

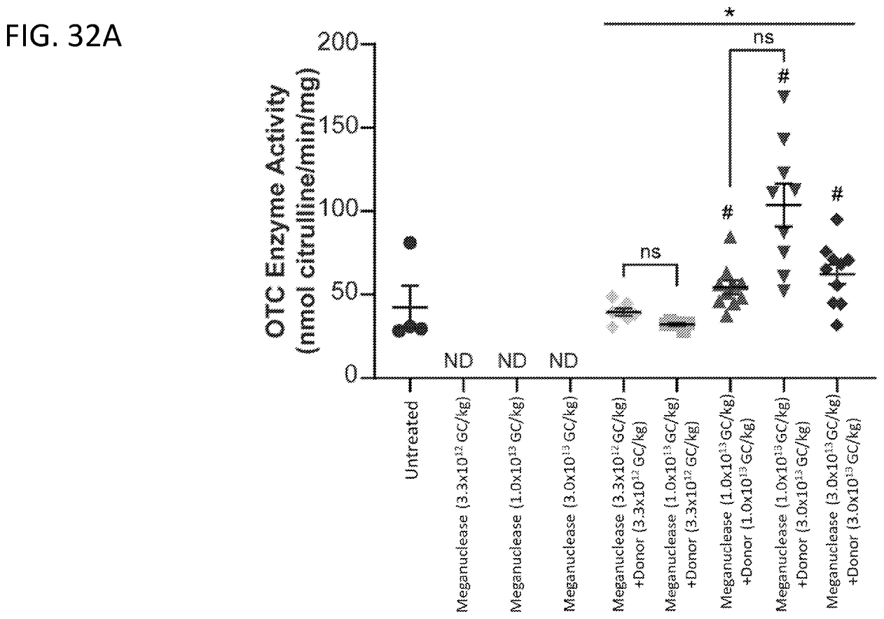

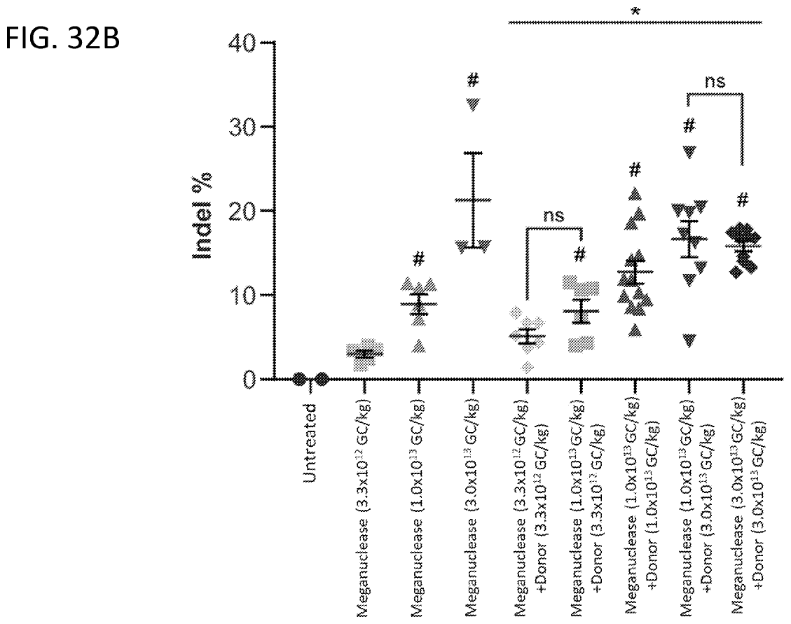

Additionally, on Day 60±2, an OTC enzyme activity assay was performed on the liver of the necropsied mice. (FIG. 32A). At each ratio, the combination treatment showed significantly more OTC enzyme activity when compared to the untreated control group. The level of on-target genome editing was calculated as the indel % from the extracted DNA by performing amplicon-seq (FIG. 32B). At each ratio, the combination treatment showed a significantly higher Indel % when compared to the untreated control group.

Furthermore, there was a statistically significant increase in OTC enzyme activity compared to the untreated control group and a statistically significant difference between the OTC enzyme activity in mice administered at the 1:1 and 1:3 ratios. After the Indel % within Pcsk9 exon 7 was determined, a dose-dependent increase was found in on-target genome editing with increasing doses of the meganuclease, either alone or in combination with donor. As expected, the indel % at the same dose of the meganuclease, either alone or in combination with donor (irrespective of the ratio), were equivalent.

In the previous examples, a 1:3 ratio of ARCUS meganuclease to donor vectors was utilized. The work described in this example identifies this 1:3 ratio as the optimal ratio to efficiently knock-in the human OTC minigene and protect against a high-protein diet in a mouse model of OTCD.

### Example 14—hFIX Mini Gene Knock-In in Pcsk9 Locus Using Coadminstration Regimen with Rituximab and M281 in Rhesus Macaques

The aim of this study was to assess the effect of knock-in of the human FIX gene in macaques of different ages via a co-administration regimen that included Rituximab and

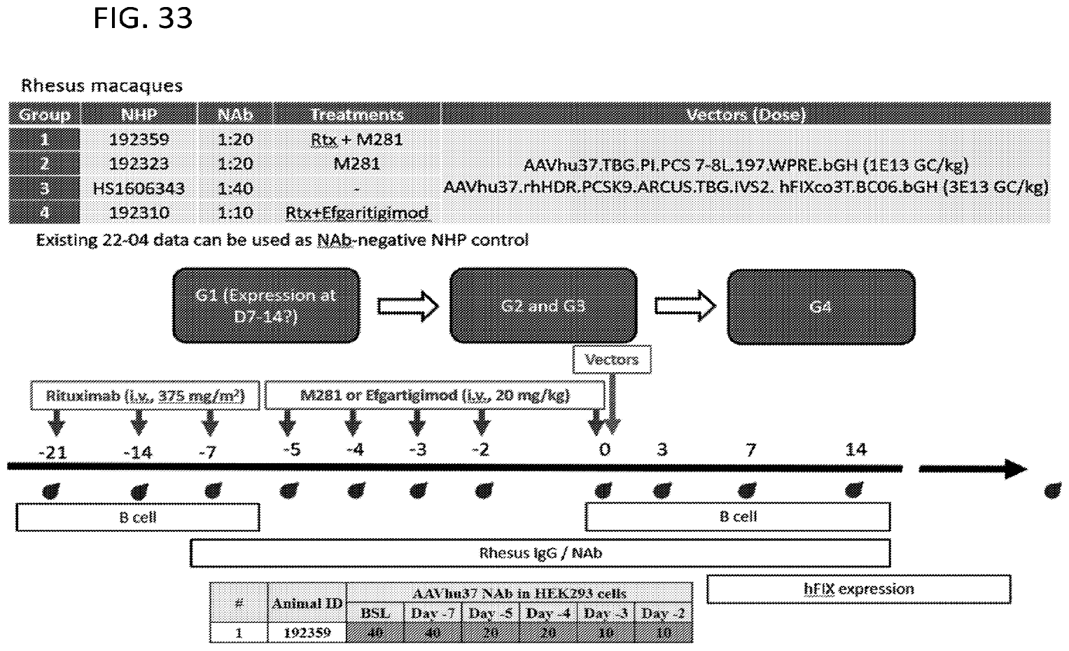

M281. The experimental design is shown in FIG. 33.

On from day −21 to day −7, rhesus macaques were IV administered 375 mg/m² rituximab. Then from days-5 to 0, the rhesus macaques were IV administered 20 mg/kg M281. Finally, on Day 0, rhesus macaques were IV co-administered an AAVhu37 vector AAVhu37.TBG.PI.PCS 7-8L. 197.WPRE.bGH, at 1.0×10¹³ GC/kg, in combination with a AAVhu37 hFIX donor vector AAVhu37.rhHDR.PCSK9.ARCUS.TBG.IVS2.hFIXco3T.BC06.bGH at 1.0×10¹³ GC/kg.

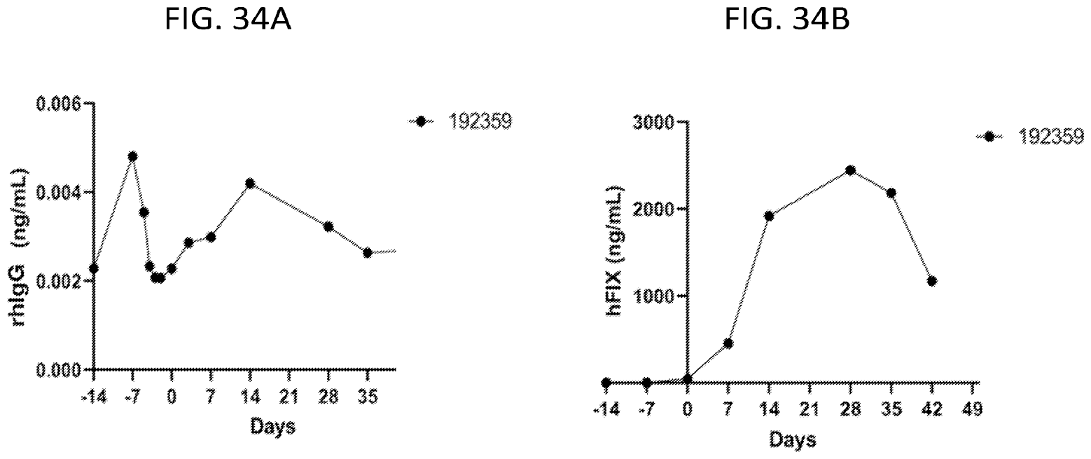

Flow cytometric analysis of leukocytes from NPH 192359 was performed on days −21, −14, −7, 0, 3, and 7. At baseline, B cells were prevalent (6.93%); however, following rituximab administration, no B cells were detected (Day-14). B cell levels recovered at day 0 (0.08%), day 3 (0.24%), day 7 (0.60%). Serum rhIgG and hFIX levels are shown in FIG. 34A and FIG. 34B, respectively.

All documents cited in this specification are incorporated herein by reference, as are sequences and text of the Sequence Listing filed herewith are incorporated by reference. While the invention has been described with reference to particular embodiments, it will be appreciated that modifications can be made without departing from the spirit of the invention. Such modifications are intended to fall within the scope of the appended claims.

What is claimed is:

1. A method of treating hemophilia B in an adolescent subject, the method comprising co-administering to the subject having hemophilia B:
(a) a gene editing AAV vector comprising a sequence encoding a nuclease and regulatory sequences that direct expression of the nuclease in a target cell comprising a PCSK9 gene; and
(b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.

2. A method of treating hemophilia B in an adult subject, the method comprising co-administering to the subject having hemophilia B:
(a) a gene editing AAV vector comprising a sequence encoding a nuclease and regulatory sequences that direct expression of the nuclease in a target cell comprising a PCSK9 gene; and
(b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.

3. The method of claim 1 or claim 2, wherein the ratio of gene editing vector to donor vector is about 1:3.

4. The method of any one of claims 1 to 3, wherein the donor AAV vector and/or gene editing AAV vector comprises an AAVhu37 capsid.

5. The method of any one of claims 1 to 4, wherein the donor AAV vector and/or gene therapy vector comprises an AA Vrh79 capsid.

6. The method of any one of claims 1 to 5, wherein the gene editing AAV vector of (a) is suspended in a vehicle for injection at a concentration of about 2×10¹¹ GC/mL to about 2×10¹² GC/mL.

7. The method of any one of claims 1 to 6, wherein the AAV donor vector of (b) is suspended in a vehicle for injection at a concentration of about 2×10¹² GC/mL to about 1×10¹³ GC/mL.

8. A method for treating hemophilia B in an adolescent subject in need thereof, the method comprising co-administering to the subject having hemophilia B:
(a) a lipid nanoparticle (LNP) comprising a mRNA sequence encoding a nuclease; and
(b) a donor AAV vector comprising a factor IX transgene and regulatory sequences which direct its expression in the target cell, the donor vector further comprising a homology-directed recombination (HDR) arms 5′ and 3′ to the transgene.

9. A method for treating hemophilia B in an adult subject in need thereof, the method comprising co-administering to the subject having hemophilia B:
(a) a lipid nanoparticle (LNP) comprising a mRNA sequence encoding a nuclease; and
(b) a donor AAV vector comprising a factor IX transgene and regulatory sequences which direct its expression in the target cell, the donor vector further comprising a homology-directed recombination (HDR) arms 5′ and 3′ to the transgene.

10. The method of any one of claims 1 to 9, wherein the donor AAV vector comprises an AAVhu37 capsid.

11. The method of any one of claims 1 to 9, wherein the donor AAV vector comprises an AAVrh79 capsid.

12. The method of any one of claims 1 to 11, wherein the nuclease targets the PCSK9 gene.

13. The method of claim 12, wherein the nuclease targets PCSK9 exon 7.

14. The method of any one of claims 1 to 13, wherein the nuclease is a meganuclease specific for PCSK9.

15. The method of claim 14, wherein the meganuclease is the ARCUS meganuclease.

16. The method of any one of claims 8 to 13, wherein the nuclease is a Cas9 nuclease and wherein said method further comprises administering an sgRNA.

17. The method of claim 16, wherein said LNP comprises the sgRNA.

18. The method of claim 16 or claim 17, wherein the Cas9 nuclease is flanked by nuclear localization signals.

19. The method of any one of claims 16 to 18, wherein the sgRNA comprises at least 20 nucleotides which specifically bind to a target site in the PCSK9 gene, said target site being 5′ to a protospacer-adjacent motif (PAM) that is specifically recognized by the Cas9.

20. The method of any one of claims 8 to 13 or 16 to 19, further comprising an RNA polymerase promoter.

21. The method of claim 20, wherein the RNA polymerase promoter is the U6 promoter.

22. The method of claim 21, wherein the U6 promoter is located 5′ of the sgRNA.

23. The method of any one of claims 16 to 22, wherein the sgRNA is 100% complementary to the target site sequence.

24. The method of any one of claims 16 to 22, wherein the sgRNA is less than 100% complementary to the target site sequence.

25. The method of any one of claims 16 to 24, wherein Cas9 is selected from Staphylococcus aureus or Streptococcus pyogenes Cas9.

26. The method of any one of claims 1 to 25, wherein the nuclease is under the control of a tissue-specific promoter.

27. The method of any one of claims 1 to 25, wherein the nuclease is under the control of a constitutive promoter.

28. The method of claim 26, wherein the nuclease is under the control of a liver-specific promoter, optionally a human thyroxin-binding globulin (TBG) promoter, or hybrid liver promoter (HLP).

29. The method of any preceding claim, wherein the gene editing AAV vector of (a) and the donor vector of (b) are delivered essentially simultaneously via the same route.

30. The method of any preceding claim, further comprising administering an anti-CD20 ligand, optionally rituximab.

31. A dual component system useful for treating hemophilia B in an adolescent subject, the system comprising:
(a) a gene editing AAV vector comprising a sequence encoding a nuclease and regulatory sequences that direct expression of the nuclease in a target cell comprising a PCSK9 gene; and
(b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.

32. A dual component system useful for treating hemophilia B in an adult subject, the method system comprising:
(a) a gene editing AAV vector comprising a sequence encoding a nuclease and regulatory sequences that direct expression of the nuclease in a target cell comprising a PCSK9 gene; and
(b) a donor AAV vector comprising a factor IX (FIX) transgene and regulatory sequences that direct expression of the transgene in the target cell, wherein the donor vector further comprises homology-directed recombination (HDR) arms 5′ and 3′ to the transgene cassette.

33. The dual component system of claim 31 or claim 32, wherein the ratio of gene editing vector to donor vector is about 1:3.

34. The dual component system of any one of claims 31 to 33, wherein the donor AAV vector and/or gene editing AAV vector comprises an AAVhu37 capsid.

35. The dual component system of any one of claims 31 to 34, wherein the donor AAV vector and/or gene therapy vector comprises an AAVrh79 capsid.

36. The dual component system of any one of claims 31 to 35, wherein the gene editing AAV vector of (a) is suspended in a vehicle for injection at a concentration of about 2×10¹¹ GC/mL to about 2×10¹² GC/mL.

37. The dual component system of any one of claims 31 to 36, wherein the AAV donor vector of (b) is suspended in a vehicle for injection at a concentration of about 2×10¹² GC/mL to about 1×10¹³ GC/mL.

38. A dual component system useful for treating hemophilia B in an adolescent subject in need thereof, the system comprising:
(a) a lipid nanoparticle (LNP) comprising a mRNA sequence encoding a nuclease; and
(b) a donor AAV vector comprising a factor IX transgene and regulatory sequences which direct its expression in the target cell, the donor vector further comprising a homology-directed recombination (HDR) arms 5′ and 3′ to the transgene.

39. A method for treating hemophilia B in an adult subject in need thereof, the method comprising co-administering to the subject having hemophilia B:
(a) a lipid nanoparticle (LNP) comprising a mRNA sequence encoding a nuclease; and
(b) a donor AAV vector comprising a factor IX transgene and regulatory sequences which direct its expression in the target cell, the donor vector further comprising a homology-directed recombination (HDR) arms 5′ and 3′ to the transgene.

40. The method of any one of claims 31 to 39, wherein the donor AAV vector comprises an AAVhu37 capsid.

41. The method of any one of claims 31 to 39, wherein the donor AAV vector comprises an AAVrh79 capsid.

42. The method of any one of claims 31 to 41, wherein the nuclease targets the PCSK9 gene.

43. The method of claim 42, wherein the nuclease targets PCSK9 exon 7.

44. The method of any one of claims 31 to 43, wherein the nuclease is a meganuclease specific for PCSK9.

45. The method of claim 44, wherein the meganuclease is the ARCUS meganuclease.

46. The method of any one of claims 31 to 43, wherein the nuclease is a Cas9 nuclease and wherein said method further comprises administering an sgRNA.

47. The method of claim 46, wherein said LNP comprises the sgRNA.

48. The method of claim 46 or claim 47, wherein the Cas9 nuclease is flanked by nuclear localization signals.

49. The method of any one of claims 46 to 48, wherein the sgRNA comprises at least 20 nucleotides which specifically bind to a target site in the PCSK9 gene, said target site being 5′ to a protospacer-adjacent motif (PAM) that is specifically recognized by the Cas9.

50. The method of any one of claims 38 to 43 or 46 to 49, further comprising an RNA polymerase promoter.

51. The method of claim 50, wherein the RNA polymerase promoter is the U6 promoter.

52. The method of claim 51, wherein the U6 promoter is located 5′ of the sgRNA.

53. The method of any one of claims 46 to 52, wherein the sgRNA is 100% complementary to the target site sequence.

54. The method of any one of claims 46 to 52, wherein the sgRNA is less than 100% complementary to the target site sequence.

55. The method of any one of claims 46 to 54, wherein Cas9 is selected from Staphylococcus aureus or Streptococcus pyogenes Cas9.

56. The method of any one of claims 31 to 55, wherein the nuclease is under the control of a tissue-specific promoter.

57. The method of any one of claims 31 to 55, wherein the nuclease is under the control of a constitutive promoter.

58. The method of claim 26, wherein the nuclease is under the control of a liver-specific promoter, optionally a human thyroxin-binding globulin (TBG) promoter, or hybrid liver promoter (HLP).

59. The method of any preceding claim, wherein the gene editing AAV vector of (a) and the donor vector of (b) are delivered essentially simultaneously via the same route.


SEQUENCE LISTING (86 sequences)

SEQ ID NO: 1 - DNA, 7429 bp, synthetic construct
  misc_feature 1..7429: note = production plasmid U6.sgR.PCSK9.ABP2.TBG-S1.hSaCas9(pX601+3NLS).bGH
  misc_feature 1..168: note = AAV ITR
  regulatory 193..199: note = terminator - U6 terminator; regulatory_class = terminator
  misc_feature 199..275: note = gRNA scaffold
  misc_feature 276..295: note = PCSK9 sgRNA
  regulatory 297..545: note = promoter - U6 promoter; regulatory_class = promoter
  regulatory 557..656: note = enhancer - alpha mic/bik enhancer; regulatory_class = enhancer
  regulatory 663..762: note = enhancer - alpha mic/bik enhancer; regulatory_class = enhancer
  regulatory 777..952: note = promoter - TBG-S1; regulatory_class = promoter
  misc_feature 956..958: note = Stop codon mutation
  misc_feature 973..1017: note = SV40 NLS
  primer_bind 1160..1179: note = SA2-Seq1
  primer_bind 1647..1666: note = SA2-Seq2
  primer_bind 2170..2189: note = SA2-Seq3
  primer_bind 2680..2700: note = SA2-Seq4
  primer_bind 3130..3149: note = SA2-Seq5
  misc_feature 4174..4221: note = NLS
  regulatory 4233..4435: note = polyA_signal - bGH poly A; regulatory_class = polyA_signal_sequence
  misc_feature 4456..4619: note = AAV ITR
  misc_feature 5382..6239: note = Amp-R
  misc_feature 6413..7001: note = Origin
  source 1..7429: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaggctatttctagctctaaaacaaaaaaatctcgccaacaagttgacgagataaacacggcattttgccttgttttagtagattctgtttccagagtactaaaacgggactttggggaccaacttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagtgaccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggcctgctgccaccaaaaaggccggccaggcaaaaaagaaaaagtaatgactagtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 2 - DNA, 4619 bp, synthetic construct
  misc_feature 1..4619: note = vector genome U6.sgR.PCSK9.ABP2.TBG-S1.hSaCas9(pX601+3NLS).bGH
  misc_feature 1..168: note = AAV ITR
  regulatory 193..199: note = terminator - U6 Terminator; regulatory_class = terminator
  misc_feature 199..275: note = shRNA scaffold
  misc_feature 276..295: note = PCSK9 sgRNA
  regulatory 297..545: note = promoter - U6 promoter; regulatory_class = promoter
  regulatory 557..656: note = enhancer - alpha mic/bik enhancer; regulatory_class = enhancer
  regulatory 663..762: note = enhancer - alpha mic/bik enhancer; regulatory_class = enhancer
  regulatory 777..952: note = promoter - TBG-S1; regulatory_class = promoter
  misc_feature 956..958: note = stop codon mutation
  misc_feature 973..1017: note = SV40 NLS
  primer_bind 1160..1179: note = Sa2-Seq1
  primer_bind 1647..1666: note = Sa2-Seq2
  primer_bind 2170..2189: note = Sa2-Seq3
  primer_bind 2680..2700: note = Sa2-Seq4
  primer_bind 3130..3149: note = Sa2-Seq5
  misc_feature 4174..4221: note = NLS
  regulatory 4233..4435: note = polyA_signal - bGH polyA; regulatory_class = polyA_signal_sequence
  misc_feature 4456..4619: note = AAV ITR
  source 1..4619: mol_type = other DNA
  CDS 967..4221: note = SaCas9 (px601+3LNS)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaggctatttctagctctaaaacaaaaaaatctcgccaacaagttgacgagataaacacggcattttgccttgttttagtagattctgtttccagagtactaaaacgggactttggggaccaacttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagtgaccgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggcctgctgccaccaaaaaggccggccaggcaaaaaagaaaaagtaatgactagtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - protein, 1085 aa, synthetic construct
  REGION 1..1085: note = Synthetic Construct
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 4 - DNA, 7507 bp, synthetic construct
  misc_feature 1..7507: note = production plasmid U6.sgR3.PCSK9.ABP2.HLP.hSaCas9(pX601+3NLS)bGH
  misc_feature 1..168: note = AAV ITR
  regulatory 193..199: note = terminator - U6 terminator; regulatory_class = terminator
  misc_feature 199..275: note = gRNA scaffold
  misc_feature 276..295: note = PCSK9 sgRNA
  regulatory 297..545: note = promoter - U6 pomoter; regulatory_class = promoter
  regulatory 557..656: note = enhancer - alpha mic/bik enhancer; regulatory_class = enhancer
  regulatory 663..762: note = enhancer - alpha mic/bik; regulatory_class = enhancer
  regulatory 777..1027: note = promoter - HLP; regulatory_class = promoter
  misc_feature 1034..4288: note = SaCas9 (px601+3NLS)
  misc_feature 1040..1084: note = SV40 NLS
  primer_bind 1227..1246: note = Sa2-Seq1
  primer_bind 1714..1733: note = Sa2-Seq2
  primer_bind 2237..2256: note = Sa2-Seq3
  primer_bind 2747..2767: note = Sa2-Seq4
  primer_bind 3197..3216: note = Sa2-Seq5
  misc_feature 4241..4288: note = NLS
  misc_feature 4253..4686: note = AAV ITR
  regulatory 4300..4502: note = polyA_signal - bGH; regulatory_class = polyA_signal_sequence
  misc_feature 5449..6306: note = Amp-R
  misc_feature 6480..7068: note = Origin
  source 1..7507: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaggctatttctagctctaaaacaaaaaaatctcgccaacaagttgacgagataaacacggcattttgccttgttttagtagattctgtttccagagtactaaaacgggactttggggaccaacttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggcctgctgccaccaaaaaggccggccaggcaaaaaagaaaaagtaatgactagtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 5 - DNA, 4686 bp, synthetic construct
  misc_feature 1..4686: note = vector genome U6.sgR3.PCSK9.ABP2.HLP.hSaCas9(pX601+3NLS)bGH
  misc_feature 1..168: note = AAV ITR
  regulatory 193..199: note = terminator - U6 Terminator; regulatory_class = terminator
  misc_feature 199..275: note = gRNA scaffold
  misc_feature 276..295: note = PCSK9 sgRNA
  regulatory 297..545: note = promoter - U6 Promoter; regulatory_class = promoter
  regulatory 557..656: note = enhancer - alpha mic/bik enahncer; regulatory_class = enhancer
  regulatory 663..762: note = enhancer - alpa mic/bik enhancer; regulatory_class = enhancer
  regulatory 777..1027: note = promoter - HLP; regulatory_class = promoter
  misc_feature 1040..1084: note = SV40 NLS
  primer_bind 1227..1246: note = Sa2-Seq1
  primer_bind 1714..1733: note = Sa2-Seq2
  primer_bind 2237..2256: note = Sa2-Seq3
  primer_bind 2747..2767: note = Sa2-Seq4
  primer_bind 3197..3216: note = Sa2-Seq5
  misc_feature 4241..4288: note = NLS
  misc_feature 4253..4686: note = AAV ITR
  regulatory 4300..4502: note = polyA_signal - bGH polyA; regulatory_class = polyA_signal_sequence
  source 1..4686: mol_type = other DNA
  CDS 1034..4288: note = SaCas9 (px601+3NLS)
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaggctatttctagctctaaaacaaaaaaatctcgccaacaagttgacgagataaacacggcattttgccttgttttagtagattctgtttccagagtactaaaacgggactttggggaccaacttcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggcctgctgccaccaaaaaggccggccaggcaaaaaagaaaaagtaatgactagtgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - protein, 1085 aa, synthetic construct; identical to SEQ ID NO: 3
  REGION 1..1085: note = Synthetic Construct

SEQ ID NO: 7 - DNA, 3255 bp, synthetic construct
  misc_feature 1..3255: note = nucleic acid sequence SaCas9 (pX601+3NLS)
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggcctgctgccaccaaaaaggccggccaggcaaaaaagaaaaag

SEQ ID NO: 8 - DNA, 20 bp, synthetic construct
  misc_feature 1..20: note = PCSK9 sgRNA
  source 1..20: mol_type = other DNA
aagttggtccccaaagtccc

SEQ ID NO: 9 - DNA, 77 bp, synthetic construct
  misc_feature 1..77: note = sgRNA scaffold
  source 1..77: mol_type = other DNA
atctcgccaacaagttgacgagataaacacggcattttgccttgttttagtagattctgtttccagagtactaaaac

SEQ ID NO: 10 - DNA, 249 bp, synthetic construct
  misc_feature 1..249: note = U6 promoter
  source 1..249: mol_type = other DNA
ggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctc

SEQ ID NO: 11 - DNA, 176 bp, synthetic construct
  misc_feature 1..176: note = TBG-S1 promoter
  source 1..176: mol_type = other DNA
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 12 - DNA, 251 bp, synthetic construct
  misc_feature 1..251: note = HLP promoter
  source 1..251: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 13 - DNA, 417 bp, synthetic construct
  misc_feature 1..417: note = rhPCSK9 genomic sequence-Exon 7 nucleic acid sequence
  misc_feature 214..397: note = exon 7
  misc_feature 256..261: note = PAM (SaCas9)
  misc_RNA 262..281: note = sgRNA-SaCas9
  misc_feature 293..314: note = PCS 7-8 taregting sequence
  misc_feature 305..306: note = Meganuclease cutting site
  source 1..417: mol_type = other DNA
cctggctatggtagggacagaggggagcaccagggcggggcaggggtgccagagttctgcctggggagtcagattttccttaggaggggacatttgaatgggacccaaacaggtgtatagcagttgttcagcccagctggcaaggcctcagtttgcttctgcaacccctctcttgggctcctttctgtgccacctacctcctcacctttccaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacggagtgggacatcgcaggctgctgcccacgtggctggtaagtcaccaccccactgc

SEQ ID NO: 14 - DNA, 8924 bp, synthetic construct
  misc_feature 1..8924: note = production plasmid TBG.hOTCco2.G4SVPA.LB
  repeat_region 1..105: note = ITR
  regulatory 164..880: note = promoter - TBG promoter; regulatory_class = promoter
  regulatory 183..282: note = enhancer - enhancer; regulatory_class = enhancer
  regulatory 847..850: note = TATA_signal - TATA signal; regulatory_class = TATA_box
  misc_feature 972..2039: note = hOTCco2
  regulatory 2042..2178: note = polyA_signal - G4SVPA; regulatory_class = polyA_signal_sequence
  repeat_region 2207..2374: note = ITR
  misc_feature 3898..5700: note = tmp1
  misc_feature 6866..7726: note = Amp-R
  misc_feature 7897..8485: note = Origin
  source 1..8924: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtggtacctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcattagccaccatgctgttcaacctgcggatcctgctgaacaacgccgccttccggaacggacacaacttcatggtgcggaacttccggtgcggacagccactgcagaacaaggtgcagctgaagggacgggacctgctgaccctgaagaacttcaccggagaggagatcaagtacatgctgtggctgagcgccgatctgaagttccggatcaagcagaagggagagtacctgccactgctgcagggaaagagcctgggaatgatcttcgagaagcggagcacccggacccggctgagcaccgagaccggattcgccctgctgggaggacacccatgcttcctgaccacccaggacatccacctgggagtgaatgagagcctgaccgacaccgcccgggtgctgagcagcatggccgacgccgtgctggcccgggtgtacaagcagagcgacctggataccctggccaaggaggccagcatcccaatcatcaacggactgagcgacctgtaccacccaatccagatcctggccgattacctgaccctgcaggagcactacagcagcctgaagggactgaccctgagctggatcggagacggaaacaacatcctgcacagcatcatgatgagcgccgccaagttcggaatgcacctgcaggccgccaccccaaagggatacgagccagatgccagcgtgaccaagctggccgagcagtacgccaaggagaacggaaccaagctgctgctgaccaacgacccactggaggccgcccacggaggaaacgtgctgatcaccgatacctggatcagcatgggacaggaggaggagaagaagaagcggctgcaggccttccagggataccaggtgaccatgaagaccgccaaggtggccgccagcgattggaccttcctgcactgcctgccacggaagccagaggaggtggacgacgaggtgttctacagcccacggagcctggtgttcccagaggccgagaaccggaagtggaccatcatggccgtgatggtgagcctgctgaccgattacagcccacagctgcagaagccaaagttctgataagcggccgctatttgtgaaatttgtgatgctattgctttatttgtaaccattataagctgcaataaacaagttaacaacaacaattgcattcattttatgtttcaggttcagggggaggtgtgggaggttttttaggcatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatccccgggtactctagttaattaactagaggatcttgatgtaatccaaggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaaggacggccggcgccgccgatctgaaatgtcccgtccggacggagaccaagcgaggagctcaccgactcgtcgttgagctgaatacctcgccctctgattgtcaggtgagttataccctgcccgggcgaccgcacgtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 15 - DNA, 2374 bp, synthetic construct
  misc_feature 1..2374: note = vector genome TBG.hOTCco2.G4SVPA.LB
  repeat_region 1..105: note = ITR
  regulatory 164..880: note = promoter - TBG promoter; regulatory_class = promoter
  regulatory 183..282: note = enhancer - enhancer; regulatory_class = enhancer
  regulatory 847..850: note = TATA_signal - TATA; regulatory_class = TATA_box
  regulatory 2042..2178: note = polyA_signal - G4SVPA; regulatory_class = polyA_signal_sequence
  repeat_region 2207..2374: note = ITR
  source 1..2374: mol_type = other DNA
  CDS 972..2039: note = hOTCco2
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcaattcacgcgtggtacctagaactatagctagaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcattagccaccatgctgttcaacctgcggatcctgctgaacaacgccgccttccggaacggacacaacttcatggtgcggaacttccggtgcggacagccactgcagaacaaggtgcagctgaagggacgggacctgctgaccctgaagaacttcaccggagaggagatcaagtacatgctgtggctgagcgccgatctgaagttccggatcaagcagaagggagagtacctgccactgctgcagggaaagagcctgggaatgatcttcgagaagcggagcacccggacccggctgagcaccgagaccggattcgccctgctgggaggacacccatgcttcctgaccacccaggacatccacctgggagtgaatgagagcctgaccgacaccgcccgggtgctgagcagcatggccgacgccgtgctggcccgggtgtacaagcagagcgacctggataccctggccaaggaggccagcatcccaatcatcaacggactgagcgacctgtaccacccaatccagatcctggccgattacctgaccctgcaggagcactacagcagcctgaagggactgaccctgagctggatcggagacggaaacaacatcctgcacagcatcatgatgagcgccgccaagttcggaatgcacctgcaggccgccaccccaaagggatacgagccagatgccagcgtgaccaagctggccgagcagtacgccaaggagaacggaaccaagctgctgctgaccaacgacccactggaggccgcccacggaggaaacgtgctgatcaccgatacctggatcagcatgggacaggaggaggagaagaagaagcggctgcaggccttccagggataccaggtgaccatgaagaccgccaaggtggccgccagcgattggaccttcctgcactgcctgccacggaagccagaggaggtggacgacgaggtgttctacagcccacggagcctggtgttcccagaggccgagaaccggaagtggaccatcatggccgtgatggtgagcctgctgaccgattacagcccacagctgcagaagccaaagttctgataagcggccgctatttgtgaaatttgtgatgctattgctttatttgtaaccattataagctgcaataaacaagttaacaacaacaattgcattcattttatgtttcaggttcagggggaggtgtgggaggttttttaggcatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 354 aa, synthetic construct
  REGION 1..354: note = Synthetic Construct
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 17 - DNA, 1068 bp, synthetic construct
  misc_feature 1..1068: note = engineered nucleic acid sequence hOTCco2
  source 1..1068: mol_type = other DNA
atgctgttcaacctgcggatcctgctgaacaacgccgccttccggaacggacacaacttcatggtgcggaacttccggtgcggacagccactgcagaacaaggtgcagctgaagggacgggacctgctgaccctgaagaacttcaccggagaggagatcaagtacatgctgtggctgagcgccgatctgaagttccggatcaagcagaagggagagtacctgccactgctgcagggaaagagcctgggaatgatcttcgagaagcggagcacccggacccggctgagcaccgagaccggattcgccctgctgggaggacacccatgcttcctgaccacccaggacatccacctgggagtgaatgagagcctgaccgacaccgcccgggtgctgagcagcatggccgacgccgtgctggcccgggtgtacaagcagagcgacctggataccctggccaaggaggccagcatcccaatcatcaacggactgagcgacctgtaccacccaatccagatcctggccgattacctgaccctgcaggagcactacagcagcctgaagggactgaccctgagctggatcggagacggaaacaacatcctgcacagcatcatgatgagcgccgccaagttcggaatgcacctgcaggccgccaccccaaagggatacgagccagatgccagcgtgaccaagctggccgagcagtacgccaaggagaacggaaccaagctgctgctgaccaacgacccactggaggccgcccacggaggaaacgtgctgatcaccgatacctggatcagcatgggacaggaggaggagaagaagaagcggctgcaggccttccagggataccaggtgaccatgaagaccgccaaggtggccgccagcgattggaccttcctgcactgcctgccacggaagccagaggaggtggacgacgaggtgttctacagcccacggagcctggtgttcccagaggccgagaaccggaagtggaccatcatggccgtgatggtgagcctgctgaccgattacagcccacagctgcagaagccaaagttctgataa

SEQ ID NO: 18 - DNA, 4687 bp, synthetic construct
  misc_feature 1..4687: note = production plasmid TBG-S1-F113.PCS7-8L.197.bGH
  repeat_region 1..168: note = ITR
  regulatory 206..318: note = promoter - TBG S1 promoter; regulatory_class = promoter
  misc_feature 330..1424: note = PCS7-8L
  regulatory 1435..1649: note = polyA_signal - bGH polyA signal; regulatory_class = polyA_signal_sequence
  repeat_region 1699..1866: note = ITR
  misc_feature 2629..3486: note = Amp-R
  misc_feature 3660..4248: note = Origin
  source 1..4687: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctttgaaaataccatcccagggttaatgctggggttaatttataactaagagtgctctagttttgcaatacaggacatgctataaaaatggaaagatgttgctttctgagagacagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 19 - DNA, 1866 bp, synthetic construct
  misc_feature 1..1866: note = vector genome TBG-S1-F113.PCS7-8L.197.bGH
  repeat_region 1..168: note = ITR
  regulatory 206..318: note = promoter - TBG S1; regulatory_class = promoter
  regulatory 1435..1649: note = polyA_signal - bGH polyA; regulatory_class = polyA_signal_sequence
  repeat_region 1691..1736: note = ITR
  source 1..1866: mol_type = other DNA
  CDS 330..1424: note = PCS7-8L
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctttgaaaataccatcccagggttaatgctggggttaatttataactaagagtgctctagttttgcaatacaggacatgctataaaaatggaaagatgttgctttctgagagacagcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtacgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - protein, 364 aa, synthetic construct
  REGION 1..364: note = Synthetic Construct
MAPKKKRKVHMNTKYNKEFLLYLAGFVDGDGSIFARIKPSQRSKFKHKLHLVFAVYQKTQRRWFLDKLVDEIGVGYVLDSGSVSFYSLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSIYARIKPVQRAKFKHELVLGFDVTQKTQRRWFLDKLVDEIGVGYVYDKGSVSAYRLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 21 - DNA, 1095 bp, synthetic construct
  misc_feature 1..1095: note = nucleic acid sequence PCS7-8L
  source 1..1095: mol_type = other DNA
atggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaa

SEQ ID NO: 22 - DNA, 2079 bp, Homo sapiens
  source 1..2079: mol_type = other DNA
  CDS 1..2079: note = PCSK9 CDS
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtga

SEQ ID NO: 23 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 24 - DNA, 132 bp, Homo sapiens
  source 1..132: mol_type = other DNA
tgggctcctttctctgccacccacctcctcacctttccaggtcatcacagttggggccaccaatgcccaagaccagccggtgaccctggggactttggggaccaactttggccgctgtgtggacctctttgc

SEQ ID NO: 25 - DNA, 137 bp, Homo sapiens
  source 1..137: mol_type = other DNA
ttgccccaggggaggacatcattggtgcctccagcgactgcagcacctgctttgtgtcacagagtgggacatcacaggctgctgcccacgtggctggtaagtcaccaccccactgcctcggccaccgtgatgctaac

SEQ ID NO: 26 - DNA, 500 bp, Homo sapiens
  source 1..500: mol_type = other DNA
acctaggtccccccggcctatcaaggcttccctggcggccgaatttaaaggcatcaagcaaacaaagcccaacacatctctgccttgtcctctcagtttccccccgtggcacttagaaccacttgatacaccgaatagtttccggtctatctcccccactaggatgtaaactccacaggggcattgggaatgctgcctggctatggtagggacagaggggagcaccagggcggggcaggggtgccagagttctgcctgggcagtcagattttccttaggaggggacatttgagtgggacccaaacaggtgtatagcagttgtccagcccagctggcaaggcctgagtctgcctctgcaacccctctcttgggctcctttctctgccacccacctcctcacctttccaggtcatcacagttggggccaccaatgcccaggaccagccggtgaccctggggactttggggaccaactttggccgctgtgtggacctctttgc

SEQ ID NO: 27 - DNA, 500 bp, Homo sapiens
  source 1..500: mol_type = other DNA
ttgccccaggggaggacatcattggtgcctccagcgactgcagcacctgctttgtgtcacagagtgggacatcacaggctgctgcccacgtggctggtaagtcaccaccccactgcctcggccaccgtgatgctaacagcccctttggcagtcagggtctgtgccgggacctccagtgccaggctctgtgcagggggaccagagatgaagtaggcctgatggtgccttcaaggacactcagtctgatgagggaggcgagtgcacagagggaacacgaggtcagggctgtattagagggagcccagaggaggcacctgcccagcccgagggtcagagaaggcatcttggaggagggacatttgatcgggagcttgatggatgaataggagtttacctggccgataagacagcaactaccaaggcttagaggtgtgagaggaggctgtcttacctcactgagtaaggactgcaggcggcttaccttcgagaagagagcttag

SEQ ID NO: 28 - DNA, 516 bp, Macaca mulatta
  source 1..516: mol_type = other DNA
aagcatacctgacagcggtaacccagatcctccctggcctcggaggcttccctggcagcccaatttaaaggcatcaagcaaacaaagcccaacacatctctgccttgccctctcagtctccccccgtggcacttagagccacctgatacaccgagtagtttcctatctccctcactagaatgtaaactccacaggggcattgggaatgctgcctggctatggtagggacagaggggagcaccagggcggggcaggggtgccagagttctgcctggggagtcagattttccttaggaggggacatttgaatgggacccaaacaggtgtatagcagttgttcagcccagctggcaaggcctcagtttgcttctgcaacccctctcttgggctcctttctgtgccacctacctcctcacctttccaggtcatcacagttggggccaccaatgcccaggaccagccggtgaccctggggactttggggaccaactttggccgctgtgtggacctctttgc

SEQ ID NO: 29 - DNA, 510 bp, Macaca mulatta
  source 1..510: mol_type = other DNA
ttgccccaggggaggacatcattggtgcctccagcgactgcagcacctgctttgtgtcacggagtgggacatcgcaggctgctgcccacgtggctggtaagtcaccaccccactgccttggccaccgtgatgctaacagccctttggcggtcagggtctgtgccaggacctccagtgccaggctctgtgcagggggaccagagatgagacaggcctgatggtgccttcatggacactcagtctgatgagggagacgagtgcacagtgggaacacgaggttagggctgtattagagggagcccagaggaggcacctgcccagcccgagggtcagagaaggcttcttggacaagggacatttgatctggagcttgatggatgaataggagtccacctggccgataagacagcaactaccaaggcttagaggtgtgacggaaggctgtcttacctcactgtgtgagggactgcaggcggcttaccttctagaagagagcttggtgtctctggt

SEQ ID NO: 30 - DNA, 1062 bp, Homo sapiens
  misc_feature 1..1062: note = CDS for hOTC
  source 1..1062: mol_type = other DNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattt

SEQ ID NO: 31 - protein, 9 aa, synthetic construct
  REGION 1..9: note = CRE motif
LAGLIDADG

SEQ ID NO: 32 - DNA, 22 bp, synthetic construct
  misc_feature 1..22: note = CRE Recognition sequence
  source 1..22: mol_type = other DNA
caaaacgtcgtgagacagtttg

SEQ ID NO: 33 - DNA, 2214 bp, adeno-associated virus rh79
  source 1..2214: mol_type = other DNA
  CDS 1..2214
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgatcccccgactcctccacgggcatcggcaaaaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaatgggacatcgggaggaagcaccaacgacaacacctacttcggctacagcaccccctgggggtattttgacttcaacagattccactgtcacttctcaccacgtgactggcagagactcatcaacaacaactggggattccggcccaagagactcagcttcaagctcttcaacatccaggttaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaacggaagtcaagccgtaggccgttcctcattctactgcctggaatattttccatctcaaatgctgcggactggaaacaactttgaatttagctacacctttgaggacgtgcccttccacagcagctacgcacacagccagagcctggaccggctgatgaaccctctcatcgaccagtacctgtattacctatccagaactcagtccacaggaggaactcaaggtacacagcaattgttattttctcaagccgggcctgcaaatatgtcggctcaggccaagaactggctacctggaccttgctaccggcagcagcgagtctccacgacactgtcgcaaaacaacaacagcaactttgcttggactggtgccacgaaatatcatctgaacggaagagactctttggtgaatcccggtgttgctatggcaacgcataaggacgacgaggaacgtttctttccatcgagcggagtcctgatgtttggaaaacagggtgctggaagagacaatgtggactatagcagcgttatgctaaccagcgaggaagaaatcaagaccactaaccctgtagccactgaacaatacggcgtggtggctgataacttgcagcaaaccaatacaggacctatcgtgggaaatgtcaacagccaaggagccttacctggcatggtctggcagaaccgagacgtgtacctgcagggtcccatttgggccaagattcctcacacggacggcaactttcacccgtctcctctgatgggcggctttggactgaaacacccgcctcctcaaatcctgatcaagaacactcccgttcctgcggatcctccaacgacgttcagccaggcgaaattggcttccttcatcacgcagtatagtaccggccaggtcagcgtggagatcgagtgggagctgcagaaggagaacagcaagcgctggaacccagaaattcagtatacttccaactactacaaatctacaaatgtggactttgctgtcaataccgagggtacatattcagagcctcgccccattggaactcgttacctcacccgtaatctg

SEQ ID NO: 34 - protein, 738 aa, adeno-associated virus rh79
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 35 - DNA, 2214 bp, adeno-associated virus 8
  source 1..2214: mol_type = other DNA
  CDS 1..2214
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 36 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 37 - DNA, 2214 bp, adeno-associated virus hu37
  source 1..2214: mol_type = other DNA
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttggtcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatatccaatgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggacgtcttcatgattccccagtacggctaccttacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcatcgaccagtacctgtactacttatccagaactcagtccacaggaggaactcaaggtacccagcaattgttattttctcaagctgggcctgcaaacatgtcggctcaggctaagaactggctacctggaccttgctaccggcagcagcgagtctctacgacactgtcgcaaaacaacaacagcaactttgcttggactggtgccaccaaatatcacctgaacggaagagactctttggtaaatcccggtgtcgccatggcaacccacaaggacgacgaggaacgcttcttcccgtcgagtggagtcctgatgttcggaaaacagggtgctggaagagacaatgtggactacagcagcgttatgctaaccagcgaagaagaaattaaaaccactaaccccgtagccacagaacaatacggtgtggtggctgacaacttgcagcaaaccaatacagggcctattgtgggaaatgtcaacagccaaggagccttacctggcatggtctggcagaaccgagacgtgtacctgcagggtcccatctgggccaagattcctcacacggacggcaacttccacccttcaccgctaatgggaggatttggactgaagcacccacctcctcagatcctgatcaagaacacgccggtacctgcggatcctccaacaacgttcagccaggcgaaattggcttccttcattacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttacctcacccgtaatctg

SEQ ID NO: 38 - protein, 738 aa, adeno-associated virus hu37
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 39 - DNA, 2217 bp, adeno-associated virus rh.90
  source 1..2217: mol_type = other DNA
  CDS 1..2217
atggctgccgatggttatcttccagattggctcgaggacaacctctctgagggcattcgcgagtggtgggacctgaaacctggagccccaaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgataatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctggaaagaagagaccggtagagccatcaccacagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtagttcctcgggaaattggcattgcgattccacatggctgggcgacagagtcatcaccaccagcacccgaacctgggccctgcccacctacaacaaccacctctacaagcaaatctccaacgggacctcgggaggcagcaccaacgacaacacctacttcggctacagcaccccctgggggtattttgactttaacagattccactgccacttctcaccacgtgactggcagcgacttatcaacaacaactggggattccggcccaagagactcagcttcaagctcttcaacatccaggtcaaggaggtcacgcaaaatgaaggcaccaagaccatcgccaataacctcaccagcaccatccaggtgtttacggactcggaataccagctgccatacgtcctcggctctgcccaccagggctgcctgcctccgttcccggcggacgtcttcatgattcctcagtatggctacctgacgctgaacaacggaagtcaggccgtgggccgttcctccttctactgcctggagtactttccctctcagatgctaagaacgggcaacaacttctccttcagctataccttcgaggacgtgcctttccacagcagctacgcgcacagccagagcctggaccggctgatgaatcccctcattgaccagtacctgtactacctgtcgcggacacaatccacaggaggcacagcgggaactcagcagttgctgttttctcaggccgggcctaacaacatgtctgctcaggccaaaaactggctacccggaccttgttatcggcagcaacgtgtttccacgacactgtcgcaaaacaacaacagcaactttgcctggaccggtgccaccaaataccacctgaacggaagagactctctggtaaatccgggtgtcgccatggcaaccaacaaggacgacgaggaccgcttcttcccatccagcggcatcctcatgtttggcaagcagggggctggaaaagacaacgtggactatagcaacgtgatgctaaccagcgaggaagaaatcaagaccactaaccctgtggccacagaacagtatggtgtggtggcggataacctgcagcagcaaaacacagctcctattgtgggggccgtcaacagccaaggagccttacctggcatggtttggcagaaccgggacgtgtacctgcagggtcccatctgggccaagattcctcacacggatggtaactttcacccgtctcctctcatgggcggctttggacttaaacatccgcctcctcagatcctgatcaagaacactcccgttcctgcggatcctccaacggcgttcaaccaggccaagctgaactctttcatcacgcagtacagcaccggacaagtcagcgtggagatcgagtgggagctgcagaaggagaacagcaagcgctggaacccagagattcagtatacctccaactactacaaatctacaaatgtggactttgctgttaatactgagggtgtttactctgagcctcgccccattggcacccgttacctcacccgtaatctgtaa

SEQ ID NO: 40 - protein, 738 aa, adeno-associated virus rh.90
MAADGYLPDWLEDNLSEGIREWWDLKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 41 - DNA, 434 bp, Homo sapiens
  misc_feature 1..434: note = TBG promoter
  source 1..434: mol_type = other DNA
tgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 42 - DNA, 3181 bp, synthetic construct
  misc_feature 1..3181: note = Constructed sequence
  repeat_region 1..130: note = 5' ITR
  regulatory 211..907: note = promoter - TBG with enhancer; regulatory_class = promoter
  regulatory 211..310: note = promoter - alpha mic/bik enhancer; regulatory_class = promoter
  regulatory 317..419: note = promoter - alpha mic/bik enhancer; regulatory_class = promoter
  regulatory 474..907: note = promoter - TBG promoter; regulatory_class = promoter
  intron 939..1071
  misc_feature 2022..2743: note = WPRE
  regulatory 2750..2964: note = polyA_signal - BGH polyA; regulatory_class = polyA_signal_sequence
  repeat_region 3052..3181: note = 3' ITR
  source 1..3181: mol_type = other DNA
  CDS 1089..2183: note = ARCUS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaccgaagaagaagcgcaaggtgcatatgaatacaaaatataataaagagttcttactctacttagcagggtttgtagacggtgacggttccatctttgccaggatcaagcctagtcaacgtagtaagttcaagcacaagctgcatctcgttttcgctgtctatcagaagacacagcgccgttggttcctcgacaagctggtggacgagatcggtgtgggttacgtgctggactctggcagcgtctccttttactcgctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaagacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcccgtatcaagccggttcagcgggctaagttcaagcacgagctggttctcgggttcgatgtcactcagaagacacagcgccgttggttcctcgacaagctggtggacgagatcggtgtgggttacgtgtatgacaagggcagcgtctccgcgtaccgtctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccctaaggtaccagcggccgatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - protein, 364 aa, synthetic construct; identical to SEQ ID NO: 20
  REGION 1..364: note = Synthetic Construct

SEQ ID NO: 44 - DNA, 184 bp, Homo sapiens
  misc_feature 80..101: note = ARCUS targeting seq
  source 1..184: mol_type = other DNA
gtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctg

SEQ ID NO: 45 - DNA, 184 bp, Macaca mulatta
  source 1..184: mol_type = other DNA
gtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacggagtgggacatcgcaggctgctgcccacgtggctg

SEQ ID NO: 46 - DNA, 184 bp, Mus musculus
  source 1..184: mol_type = other DNA
gtcatcacagtcggggccacgaatgcccaggaccagccagttaccttggggactttggggactaattttggacgctgtgtggatctctttgcccccgggaaggacatcatcggagcgtccagtgactgcagcacatgcttcatgtcacagagtgggacctcacaggctgctgcccacgtggccg

SEQ ID NO: 47 - DNA, 1179 bp, Homo sapiens
  source 1..1179: mol_type = other DNA
atggcctctcacaagctgctggtgaccccccccaaggccctgctcaagcccctctccatccccaaccagctcctgctggggcctggtccttccaacctgcctcctcgcatcatggcagccggggggctgcagatgatcgggtccatgagcaaggatatgtaccagatcatggacgagatcaaggaaggcatccagtacgtgttccagaccaggaacccactcacactggtcatctctggctcgggacactgtgccctggaggccgccctggtcaatgtgctggagcctggggactccttcctggttggggccaatggcatttgggggcagcgagccgtggacatcggggagcgcataggagcccgagtgcacccgatgaccaaggaccctggaggccactacacactgcaggaggtggaggagggcctggcccagcacaagccagtgctgctgttcttaacccacggggagtcgtccaccggcgtgctgcagccccttgatggcttcggggaactctgccacaggtacaagtgcctgctcctggtggattcggtggcatccctgggcgggacccccctttacatggaccggcaaggcatcgacatcctgtactcgggctcccagaaggccctgaacgcccctccagggacctcgctcatctccttcagtgacaaggccaaaaagaagatgtactcccgcaagacgaagcccttctccttctacctggacatcaagtggctggccaacttctggggctgtgacgaccagcccaggatgtaccatcacacaatccccgtcatcagcctgtacagcctgagagagagcctggccctcattgcggaacagggcctggagaacagctggcgccagcaccgcgaggccgcggcgtatctgcatgggcgcctgcaggcactggggctgcagctcttcgtgaaggacccggcgctccggcttcccacagtcaccactgtggctgtacccgctggctatgactggagagacatcgtcagctacgtcatagaccacttcgacattgagatcatgggtggccttgggccctccacggggaaggtgctgcggatcggcctgctgggctgcaatgccacccgcgagaatgtggaccgcgtgacggaggccctgagggcggccctgcagcactgccccaagaagaagctgtga

SEQ ID NO: 48 - protein, 392 aa, Homo sapiens
MASHKLLVTPPKALLKPLSIPNQLLLGPGPSNLPPRIMAAGGLQMIGSMSKDMYQIMDEIKEGIQYVFQTRNPLTLVISGSGHCALEAALVNVLEPGDSFLVGANGIWGQRAVDIGERIGARVHPMTKDPGGHYTLQEVEEGLAQHKPVLLFLTHGESSTGVLQPLDGFGELCHRYKCLLLVDSVASLGGTPLYMDRQGIDILYSGSQKALNAPPGTSLISFSDKAKKKMYSRKTKPFSFYLDIKWLANFWGCDDQPRMYHHTIPVISLYSLRESLALIAEQGLENSWRQHREAAAYLHGRLQALGLQLFVKDPALRLPTVTTVAVPAGYDWRDIVSYVIDHFDIEIMGGLGPSTGKVLRIGLLGCNATRENVDRVTEALRAALQHCPKKKL

SEQ ID NO: 49 - DNA, 1113 bp, Homo sapiens
  source 1..1113: mol_type = other DNA
atgctcccccggctaatttgtatcaatgattatgaacaacatgctaaatcagtacttccaaagtctatatatgactattacaggtctggggcaaatgatgaagaaactttggctgataatattgcagcattttccagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggatgaaaaattttgaaaccagtactttatcattttctcctgaggaaaattttggagacgacagtggacttgctgcatatgtggctaaagcaatagacccatctatcagctgggaagatatcaaatggctgagaagactgacatcattgccaattgttgcaaagggcattttgagaggtgatgatgccagggaggctgttaaacatggcttgaatgggatcttggtgtcgaatcatggggctcgacaactcgatggggtgccagccactattgatgttctgccagaaattgtggaggctgtggaagggaaggtggaagtcttcctggacgggggtgtgcggaaaggcactgatgttctgaaagctctggctcttggcgccaaggctgtgtttgtggggagaccaatcgtttggggcttagctttccagggggagaaaggtgttcaagatgtcctcgagatactaaaggaagaattccggttggccatggctctgagtgggtgccagaatgtgaaagtcatcgacaagacattggtgaggaaaaatcctttggccgtttccaagatctga

SEQ ID NO: 50 - protein, 370 aa, Homo sapiens
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALSGCQNVKVIDKTLVRKNPLAVSKI

SEQ ID NO: 51 - DNA, 1611 bp, Homo sapiens
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 52 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 53 - DNA, 7056 bp, Homo sapiens
  source 1..7056: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 54 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 55 - DNA, 1386 bp, Homo sapiens
  source 1..1386: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 56 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 57 - DNA, 1395 bp, Homo sapiens
  source 1..1395: mol_type = other DNA
atggcctcggagagtgggaagctttggggtggccggtttgtgggtgcagtggaccccatcatggagaagttcaacgcgtccattgcctacgaccggcacctttgggaggtggatgttcaaggcagcaaagcctacagcaggggcctggagaaggcagggctcctcaccaaggccgagatggaccagatactccatggcctagacaaggtggctgaggagtgggcccagggcaccttcaaactgaactccaatgatgaggacatccacacagccaatgagcgccgcctgaaggagctcattggtgcaacggcagggaagctgcacacgggacggagccggaatgaccaggtggtcacagacctcaggctgtggatgcggcagacctgctccacgctctcgggcctcctctgggagctcattaggaccatggtggatcgggcagaggcggaacgtgatgttctcttcccggggtacacccatttgcagagggcccagcccatccgctggagccactggattctgagccacgccgtggcactgacccgagactctgagcggctgctggaggtgcggaagcggatcaatgtcctgcccctggggagtggggccattgcaggcaatcccctgggtgtggaccgagagctgctccgagcagaactcaactttggggccatcactctcaacagcatggatgccactagtgagcgggactttgtggccgagttcctgttctgggcttcgctgtgcatgacccatctcagcaggatggccgaggacctcatcctctactgcaccaaggaattcagcttcgtgcagctctcagatgcctacagcacgggaagcagcctgatgccccagaagaaaaaccccgacagtttggagctgatccggagcaaggctgggcgtgtgtttgggcggtgtgccgggctcctgatgaccctcaagggacttcccagcacctacaacaaagacttacaggaggacaaggaagctgtgtttgaagtgtcagacactatgagtgccgtgctccaggtggccactggcgtcatctctacgctgcagattcaccaagagaacatgggacaggctctcagccccgacatgctggccactgaccttgcctattacctggtccgcaaagggatgccattccgccaggcccacgaggcctccgggaaagctgtgttcatggccgagaccaagggggtcgccctcaaccagctgtcactgcaggagctgcagaccatcagccccctgttctcgggcgacgtgatctgcgtgtgggactacgggcacagtgtggagcagtatggtgccctgggcggcactgcgcgctccagcgtcgactggcagatccgccaggtgcgggcgctactgcaggcacagcaggcctag

SEQ ID NO: 58 - protein, 464 aa, Homo sapiens
MASESGKLWGGRFVGAVDPIMEKFNASIAYDRHLWEVDVQGSKAYSRGLEKAGLLTKAEMDQILHGLDKVAEEWAQGTFKLNSNDEDIHTANERRLKELIGATAGKLHTGRSRNDQVVTDLRLWMRQTCSTLSGLLWELIRTMVDRAEAERDVLFPGYTHLQRAQPIRWSHWILSHAVALTRDSERLLEVRKRINVLPLGSGAIAGNPLGVDRELLRAELNFGAITLNSMDATSERDFVAEFLFWASLCMTHLSRMAEDLILYCTKEFSFVQLSDAYSTGSSLMPQKKNPDSLELIRSKAGRVFGRCAGLLMTLKGLPSTYNKDLQEDKEAVFEVSDTMSAVLQVATGVISTLQIHQENMGQALSPDMLATDLAYYLVRKGMPFRQAHEASGKAVFMAETKGVALNQLSLQELQTISPLFSGDVICVWDYGHSVEQYGALGGTARSSVDWQIRQVRALLQAQQA

SEQ ID NO: 59 - DNA, 1074 bp, Homo sapiens
  source 1..1074: mol_type = other DNA
atggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaa

SEQ ID NO: 60 - protein, 359 aa, Homo sapiens
MRMEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVAETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLSSIVASLVLLHVFDSLKPPSQVELVFYVLSFCKSAVVPLASVSVIPYCLAQVLGQPHKKSL

SEQ ID NO: 61 - DNA, 1503 bp, Homo sapiens
  source 1..1503: mol_type = other DNA
atggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctggggagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctga

SEQ ID NO: 62 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 63 - DNA, 1140 bp, Homo sapiens
  source 1..1140: mol_type = other DNA
atgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctga

SEQ ID NO: 64 - protein, 379 aa, Homo sapiens
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

SEQ ID NO: 65 - DNA, 1656 bp, Homo sapiens
  source 1..1656: mol_type = other DNA
atgccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactacgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaagtga

SEQ ID NO: 66 - protein, 551 aa, Homo sapiens
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 67 - DNA, 1428 bp, Homo sapiens
  source 1..1428: mol_type = other DNA
atggagagcaaagccctgctcgtgctgactctggccgtgtggctccagagtctgaccgcctcccgcggaggggtggccgccgccgaccaaagaagagattttatcgacatcgaaagtaaatttgccctaaggacccctgaagacacagctgaggacacttgccacctcattcccggagtagcagagtccgtggctacctgtcatttcaatcacagcagcaaaaccttcatggtgatccatggctggacggtaacaggaatgtatgagagttgggtgccaaaacttgtggccgccctgtacaagagagaaccagactccaatgtcattgtggtggactggctgtcacgggctcaggagcattacccagtgtccgcgggctacaccaaactggtgggacaggatgtggcccggtttatcaactggatggaggaggagtttaactaccctctggacaatgtccatctcttgggatacagccttggagcccatgctgctggcattgcaggaagtctgaccaataagaaagtcaacagaattactggcctcgatccagctggacctaactttgagtatgcagaagccccgagtcgtctttctcctgatgatgcagattttgtagacgtcttacacacattcaccagagggtcccctggtcgaagcattggaatccagaaaccagttgggcatgttgacatttacccgaatggaggtacttttcagccaggatgtaacattggagaagctatccgcgtgattgcagagagaggacttggagatgtggaccagctagtgaagtgctcccacgagcgctccattcatctcttcatcgactctctgttgaatgaagaaaatccaagtaaggcctacaggtgcagttccaaggaagcctttgagaaagggctctgcttgagttgtagaaagaaccgctgcaacaatctgggctatgagatcaataaagtcagagccaaaagaagcagcaaaatgtacctgaagactcgttctcagatgccctacaaagtcttccattaccaagtaaagattcatttttctgggactgagagtgaaacccataccaatcaggcctttgagatttctctgtatggcaccgtggccgagagtgagaacatcccattcactctgcctgaagtttccacaaataagacctactccttcctaatttacacagaggtagatattggagaactactcatgttgaagctcaaatggaagagtgattcatactttagctggtcagactggtggagcagtcccggcttcgccattcagaagatcagagtaaaagcaggagagactcagaaaaaggtgatcttctgttctagggagaaagtgtctcatttgcagaaaggaaaggcacctgcggtatttgtgaaatgccatgacaagtctctgaataagaagtcaggctga

SEQ ID NO: 68 - protein, 475 aa, Homo sapiens
MESKALLVLTLAVWLQSLTASRGGVAAADQRRDFIDIESKFALRTPEDTAEDTCHLIPGVAESVATCHFNHSSKTFMVIHGWTVTGMYESWVPKLVAALYKREPDSNVIVVDWLSRAQEHYPVSAGYTKLVGQDVARFINWMEEEFNYPLDNVHLLGYSLGAHAAGIAGSLTNKKVNRITGLDPAGPNFEYAEAPSRLSPDDADFVDVLHTFTRGSPGRSIGIQKPVGHVDIYPNGGTFQPGCNIGEAIRVIAERGLGDVDQLVKCSHERSIHLFIDSLLNEENPSKAYRCSSKEAFEKGLCLSCRKNRCNNLGYEINKVRAKRSSKMYLKTRSQMPYKVFHYQVKIHFSGTESETHTNQAFEISLYGTVAESENIPFTLPEVSTNKTYSFLIYTEVDIGELLMLKLKWKSDSYFSWSDWWSSPGFAIQKIRVKAGETQKKVIFCSREKVSHLQKGKAPAVFVKCHDKSLNKKSG

SEQ ID NO: 69 - DNA, 1239 bp, Homo sapiens
  source 1..1239: mol_type = other DNA
atgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacacgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatag

SEQ ID NO: 70 - protein, 412 aa, Homo sapiens
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 71 - DNA, 1359 bp, Homo sapiens
  source 1..1359: mol_type = other DNA
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 72 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 73 - DNA, 2583 bp, Homo sapiens
  source 1..2583: mol_type = other DNA
atggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtga

SEQ ID NO: 74 - protein, 860 aa, Homo sapiens [sequence omitted]

SEQ ID NO: 75 - DNA, 1290 bp, Homo sapiens
  source 1..1290: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 76 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 77 - DNA, 1257 bp, Homo sapiens
  source 1..1257: mol_type = other DNA
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataa

SEQ ID NO: 78 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 79 - DNA, 2859 bp, Homo sapiens
  source 1..2859: mol_type = other DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 80 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 81 - DNA, 1386 bp, synthetic construct
  misc_feature 1..1386: note = constructed sequence
  source 1..1386: mol_type = other DNA
atgcagcgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaacggcggcagctgcaaggacgacatcaacagctacgagtgctggtgccccttcggcttcgagggcaagaactgcgagctggacgtgacctgcaacatcaagaacggcaggtgcgagcagttctgcaagaacagcgccgacaacaaggtcgtgtgctcctgcaccgagggctacagactggccgagaaccagaagtcctgcgagcccgccgtgcctttcccttgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggacgccaagcctggacagttcccctggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggagtgcgccatgaaggggaagtacggcatctacaccaaggtgtccagatacgtgaactggatcaaagaaaagaccaagctgacatga

SEQ ID NO: 82 - protein, 461 aa, Homo sapiens; identical to SEQ ID NO: 56

SEQ ID NO: 83 - DNA, 4495 bp, synthetic construct
  source 1..4495: mol_type = other DNA; note = alpha mic/bic enhancer
  misc_feature 1..168: note = AAV ITR
  misc_recomb 178..693: note = ARCUS rhesus L-ARM
  misc_feature 681..693: note = ARCUS target overlap
  regulatory 700..799: regulatory_class = enhancer
  regulatory 920..1396: note = TBG promoter; regulatory_class = promoter
  intron 1475..2046: note = human beta globin IVS2
  CDS 2071..3456: note = hFIXco3T; protein_id = 84; translation = MQRVNMIMAESPGLITICLLGYLLSAECTVFLDHENANKILNRP KRYNSGKLEEFVQGNLERECMEEKCSFEEAREVFENTERTTEFWKQYVDGDQCESNPC LNGGSCKDDINSYECWCPFGFEGKNCELDVTCNIKNGRCEQFCKNSADNKVVCSCTEG YRLAENQKSCEPAVPFPCGRVSVSQTSKLTRAETVFPDVDYVNSTEAETILDNITQST QSFNDFTRVVGGEDAKPGQFPWQVVLNGKVDAFCGGSIVNEKWIVTAAHCVETGVKIT VVAGEHNIEETEHTEQKRNVIRIIPHHNYNAAINKYNHDIALLELDEPLVLNSYVTPI CIADKEYTNIFLKFGSGYVSGWGRVFHKGRSALVLQYLRVPLVDRATCLRSTKFTIYN NMFCAGFHEGGRDSCQGDSGGPHVTEVEGTSFLTGIISWGEECAMKGKYGIYTKVSRY VNWIKEKTKLT
  polyA_site 3549..3802: note = bGH PolyA
  misc_recomb 3809..4318: note = ARCUS rhesus R-ARM
  misc_feature 3809..3821: note = ARCUS target site
  misc_feature 4328..4495: note = AAV ITR
  misc_feature 3466..3471: note = BC06
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaaacgcgtaagcatacctgacagcggtaacccagatcctccctggcctcggaggcttccctggcagcccaatttaaaggcatcaagcaaacaaagcccaacacatctctgccttgccctctcagtctccccccgtggcacttagagccacctgatacaccgagtagtttcctatctccctcactagaatgtaaactccacaggggcattgggaatgctgcctggctatggtagggacagaggggagcaccagggcggggcaggggtgccagagttctgcctggggagtcagattttccttaggaggggacatttgaatgggacccaaacaggtgtatagcagttgttcagcccagctggcaaggcctcagtttgcttctgcaacccctctcttgggctcctttctgtgccacctacctcctcacctttccaggtcatcacagttggggccaccaatgcccaggaccagccggtgaccctggggactttggggaccaactttggccgctgtgtggacctctttgcgctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgatctcgagtaactgagccgccaccatgcagcgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaacggcggcagctgcaaggacgacatcaacagctacgagtgctggtgccccttcggcttcgagggcaagaactgcgagctggacgtgacctgcaacatcaagaacggcaggtgcgagcagttctgcaagaacagcgccgacaacaaggtcgtgtgctcctgcaccgagggctacagactggccgagaaccagaagtcctgcgagcccgccgtgcctttcccttgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggacgccaagcctggacagttcccctggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggagtgcgccatgaaggggaagtacggcatctacaccaaggtgtccagatacgtgaactggatcaaagaaaagaccaagctgacatgataaaagcttgccaatgaattcgtcgacctcgaatcgaattcctgcagcccgggggatccactagttctagagcggccaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggcaattgttgccccaggggaggacatcattggtgcctccagcgactgcagcacctgctttgtgtcacggagtgggacatcgcaggctgctgcccacgtggctggtaagtcaccaccccactgccttggccaccgtgatgctaacagccctttggcggtcagggtctgtgccaggacctccagtgccaggctctgtgcagggggaccagagatgagacaggcctgatggtgccttcatggacactcagtctgatgagggagacgagtgcacagtgggaacacgaggttagggctgtattagagggagcccagaggaggcacctgcccagcccgagggtcagagaaggcttcttggacaagggacatttgatctggagcttgatggatgaataggagtccacctggccgataagacagcaactaccaaggcttagaggtgtgacggaaggctgtcttacctcactgtgtgagggactgcaggcggcttaccttctagaagagagcttggtgtctctggtccatggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 84 - protein, 461 aa, synthetic construct; identical to SEQ ID NO: 56

SEQ ID NO: 85 - DNA, 3181 bp, synthetic construct; identical to SEQ ID NO: 42
  source 1..3181: mol_type = other DNA
  misc_feature 1..168: note = AAV ITR
  misc_feature 211..907: note = TBG promoter with enhancers
  regulatory 211..310: note = alpha mic/bik enhancer; regulatory_class = enhancer
  regulatory 317..416: note = alpha mic/bik enhancer; regulatory_class = enhancer
  intron 939..1071: note = SV40 misc intron
  CDS 1089..2183: note = PCS 7-8L.197; protein_id = 86; translation = MAPKKKRKVHMNTKYNKEFLLYLAGFVDGDGSIFARIKPSQRSK FKHKLHLVFAVYQKTQRRWFLDKLVDEIGVGYVLDSGSVSFYSLSEIKPLHNFLTQLQ PFLKLKQKQANLVLKIIEQLPSAKESPDKFLEVCTWVDQIAALNDSKTRKTTSETVRA VLDSLPGSVGGLSPSQASSAASSASSSPGSGISEALRAGAGSGTGYNKEFLLYLAGFV DGDGSIYARIKPVQRAKFKHELVLGFDVTQKTQRRWFLDKLVDEIGVGYVYDKGSVSA YRLSQIKPLHNFLTQLQPFLKLKQKQANLVLKIIEQLPSAKESPDKFLEVCTWVDQIA ALNDSKTRKTTSETVRAVLDSLSEKKKSSP
  transit_peptide 1095..1115: note = nuclear localization signal of SV40 large T antigen
  regulatory 2202..2743: note = WPRE; regulatory_class = transcriptional_cis_regulatory_region
  polyA_site 2750..2964: note = BGH pA
  misc_feature 3014..3181: note = AAV ITR

SEQ ID NO: 86 - protein, 364 aa, synthetic construct; identical to SEQ ID NO: 20